(19) United States
(12) Patent Application Publication
(10) Pub. No.: US 2026/0335320 A1
(43) Pub. Date: Oct. 29, 2026

(54) ANTI-VIRULENCE AGENTS AND METHODS FOR THE TREATMENT OF PATHOGENIC BACTERIUM

(71) Applicant: The Children's Medical Center Corporation, Boston, MA (US)

(72) Inventors: Matthew M. Schaefers, Boston, MA (US); Gregory P. Priebe, Boston, MA (US)

(73) Assignee: The Children's Medical Center Corporation, Boston, MA (US)

(21) Appl. No.: 19/691,013

(22) Filed: May 28, 2026

Related U.S. Application Data
(63) Continuation of application No. PCT/US2024/057701, filed on Nov. 27, 2024.
(60) Provisional application No. 63/605,156, filed on Dec. 1, 2023.

(51) Int. Cl. A61K 31/517; A61K 31/136; A61K 31/4184; A61K 31/4245; A61K 31/4375; A61K 31/5025; A61K 31/519; A61P 31/04

(52) U.S. Cl. CPC A61K 31/517; A61K 31/136; A61K 31/4184; A61K 31/4245; A61K 31/4375; A61K 31/5025; A61K 31/519; A61P 31/04

(57) ABSTRACT

This disclosure relates to agents and methods useful for inhibiting and/or reducing the virulence of pathogenic bacterial cells, and for treating infections associated with such bacterial cells.

### CROSS REFERENCE TO RELATED APPLICATIONS

The present application is a continuation under 35 U.S.C. § 111 (a) of PCT International Patent Application No. PCT/US2024/057701, filed Nov. 27, 2024, designating the United States and published in English, which claims priority to and the benefit of U.S. Application No. 63/605,156, filed Dec. 1, 2023, which is hereby incorporated by reference in its entirety.

### SEQUENCE LISTING

The instant application contains a Sequence Listing which has been submitted electronically in XML format and is hereby incorporated by reference in its entirety. Said XML copy, created on Dec. 9, 2024, is named 167705-033401PCT_SL.xml and is 56,572 bytes in size.

### STATEMENT OF RIGHTS TO INVENTIONS MADE UNDER FEDERALLY SPONSORED RESEARCH

This invention was made with government support under grant No. AI159211, awarded by the National Institutes of Health. The government has certain rights in the invention.

### BACKGROUND OF THE INVENTION

Members of the Burkholderia genus can cause serious, difficult to treat infections. Among Burkholderia species that cause serious infections in humans are the Burkholderia cepacia complex (BCC) and Burkholderia pseudomallei. The BCC is composed of approximately 20 different species and are of major concern to people with cystic fibrosis (CF) and chronic granulomatous disease (CGD) and to an ever-growing group of hospitalized patients exposed to BCC in contaminated medications or medical devices. B. pseudomallei causes melioidosis, a serious systemic infection that can include sepsis, pneumonia, fever, and abscesses. Melioidosis is life-threatening, and mortality can be as high is 50%. B. pseudomallei is also a potential bioterrorism agent and is classified as a Tier 1 agent by the U.S. Centers for Disease Control and Prevention. Although most commonly found in tropical and sub-tropical soil in South-East Asia and Australia, B. pseudomallei was recently isolated from soil in Mississippi and Texas, and several infections occurred as a result of exposure to this soil. B. pseudomallei also recently caused a cluster of 4 cases of melioidosis in the U.S. associated with contaminated aromatherapy spray. Therefore, there is a need for new agents and methods for treating infections involving members of the Burkholderia genus, as well as other bacteria.

### SUMMARY OF THE INVENTION

As described below, the present disclosure features compositions and methods for reducing or inhibiting the virulence of members of the Burkholderia genus and other bacteria (e.g., Pseudomonas, Mycobacterium).

In an aspect, the present disclosure provides a method of reducing the virulence of a bacterial cell. The method involves: contacting a bacterial cell with an agent, where the agent is: 6-Hexyl-7-Hydroxy-3-[5-(4-Methylphenyl)-1,3,4-Oxadiazol-2-Yl]-2h-Chromen-2-One; 3-(6,7-Dimethoxy-1-{[(2-Methoxyphenyl)Carbamoyl]Methyl}-2,4-Dioxo-1,2,3,4-Tetrahydroquinazolin-3-Yl)-N-(2-Methoxyethyl)Propanamide; 1-(2-Ethylphenyl)-4-[1-(4-Phenoxybutyl)-1h-1,3-Benzodiazol-2-Yl]Pyrrolidin-2-One; 3-[2-(Azepan-1-Yl)-2-Oxoethyl]-5-[(3-Chlorophenyl)Methyl]-3h,4h,5h-Pyridazino[4,5-B]Indol-4-One; 3-[(3-Chloro-4-Methoxyphenyl)Amino]-2-Phenyl-1h-Inden-1-One; 2-[(1e)-2-[4-(Dimethylamino)Phenyl]Ethenyl]-3-(2-Methylphenyl)-3,4-Dihydroquinazolin-4-One; N-{3-[Butyl(Methyl)Amino]Propyl}-2-{5-[(3-Chlorophenyl)Methyl]-4-Oxo-3h,4h,5h-Pyridazino[4,5-B]Indol-3-Yl}Acetamide; and/or 2-Benzoyl-6-Ethyl-5h,6h,7h,8h-Thieno[2,3-B]1,6-Naphthyridin-3-Amine, where the method reduces the virulence of the bacterial cell.

In another aspect, the present disclosure provides a method of altering activity of a two-component system in a bacterial cell. The method involves: contacting the bacterial cell with an agent, where the agent is 6-Hexyl-7-Hydroxy-3-[5-(4-Methylphenyl)-1,3,4-Oxadiazol-2-Yl]-2h-Chromen-2-One; 3-(6,7-Dimethoxy-1-{[(2-Methoxyphenyl)Carbamoyl]Methyl}-2,4-Dioxo-1,2,3,4-Tetrahydroquinazolin-3-Yl)-N-(2-Methoxyethyl)Propanamide; 1-(2-Ethylphenyl)-4-[1-(4-Phenoxybutyl)-1h-1,3-Benzodiazol-2-Yl]Pyrrolidin-2-One; 3-[2-(Azepan-1-Yl)-2-Oxoethyl]-5-[(3-Chlorophenyl)Methyl]-3h,4h,5h-Pyridazino[4,5-B]Indol-4-One; 3-[(3-Chloro-4-Methoxyphenyl)Amino]-2-Phenyl-1h-Inden-1-One; 2-[(1e)-2-[4-(Dimethylamino)Phenyl]Ethenyl]-3-(2-Methylphenyl)-3,4-Dihydroquinazolin-4-One; N-{3-[Butyl(Methyl)Amino]Propyl}-2-{5-[(3-Chlorophenyl)Methyl]-4-Oxo-3h,4h,5h-Pyridazino[4,5-B]Indol-3-Yl}Acetamide; and/or 2-Benzoyl-6-Ethyl-5h,6h,7h,8 h-Thieno[2,3-B]1,6-Naphthyridin-3-Amine, thereby altering activity of the two-component system in the bacterial cell.

In another aspect, the present disclosure provides a method of reducing cell death in response to bacterial infection. The method involves: contacting a bacterial cell with an agent, where the agent is 6-Hexyl-7-Hydroxy-3-[5-(4-Methylphenyl)-1,3,4-Oxadiazol-2-Yl]-2h-Chromen-2-One; 3-(6,7-Dimethoxy-1-{[(2-Methoxyphenyl)Carbamoyl]Methyl}-2,4-Dioxo-1,2,3,4-Tetrahydroquinazolin-3-Yl)-N-(2-Methoxyethyl)Propanamide; 1-(2-Ethylphenyl)-4-[1-(4-Phenoxybutyl)-1h-1,3-Benzodiazol-2-Yl]Pyrrolidin-2-One; 3-[2-(Azepan-1-Yl)-2-Oxoethyl]-5-[(3-Chlorophenyl)Methyl]-3h,4h,5h-Pyridazino[4,5-B]Indol-4-One; 3-[(3-Chloro-4-Methoxyphenyl)Amino]-2-Phenyl-1h-Inden-1-One; 2-[(1e)-2-[4-(Dimethylamino)Phenyl]Ethenyl]-3-(2-Methylphenyl)-3,4-Dihydroquinazolin-4-One; N-{3-[Butyl(Methyl)Amino]Propyl}-2-{5-[(3-Chlorophenyl)Methyl]-4-Oxo-3h,4h,5h-Pyridazino[4,5-B]Indol-3-Yl}Acetamide; and/or 2-Benzoyl-6-Ethyl-5h,6h,7h,8 h-Thieno[2,3-B]1,6-Naphthyridin-3-Amine, where the method reduces cell death in response to infection by the bacterial cell.

In another aspect, the present disclosure provides a method of reducing the virulence of a Burkholderia bacterium, the method comprising contacting the Burkholderia bacterium with 6-Hexyl-7-Hydroxy-3-[5-(4-Methylphenyl)-1,3,4-Oxadiazol-2-Yl]-2h-Chromen-2-One, thereby reducing the virulence of the Burkholderia bacterium.

In another aspect, the present disclosure provides a method of treating a subject having a bacterial infection. The method involves: administering to the subject an agent, where the agent is: 6-Hexyl-7-Hydroxy-3-[5-(4-Methylphenyl)-1,3,4-Oxadiazol-2-Yl]-2h-Chromen-2-One; 3-(6,7-Dimethoxy-1-{[(2-Methoxyphenyl)Carbamoyl]Methyl}-2,4-Dioxo-1,2,3,4-Tetrahydroquinazolin-3-Yl)-N-(2-Methoxyethyl)Propanamide; 1-(2-Ethylphenyl)-4-[1-(4-Phenoxybutyl)-1h-1,3-Benzodiazol-2-Yl]Pyrrolidin-2-One; 3-[2-(Azepan-1-Yl)-2-Oxoethyl]-5-[(3-Chlorophenyl)Methyl]-3h,4h,5h-Pyridazino[4,5-B]Indol-4-One; 3-[(3-Chloro-4-Methoxyphenyl)Amino]-2-Phenyl-1h-Inden-1-One; 2-[(1e)-2-[4-(Dimethylamino)Phenyl]Ethenyl]-3-(2-Methylphenyl)-3,4-Dihydroquinazolin-4-One; N-{3-[Butyl(Methyl)Amino]Propyl}-2-{5-[(3-Chlorophenyl)Methyl]-4-Oxo-3h,4h,5h-Pyridazino[4,5-B]Indol-3-Yl}Acetamide; and/or 2-Benzoyl-6-Ethyl-5h,6h,7h,8 h-Thieno[2,3-B]1,6-Naphthyridin-3-Amine, thereby treating the infection.

In another aspect, the present disclosure provides a method of treating a Burkholderia infection in a subject having cystic fibrosis or chronic granulomatous disease. The method involves administering to the subject 6-Hexyl-7-Hydroxy-3-[5-(4-Methylphenyl)-1,3,4-Oxadiazol-2-Yl]-2h-Chromen-2-One, thereby treating the infection.

In another aspect, the present disclosure provides a kit for reducing or inhibiting the virulence of a bacterial cell. The kit includes an agent, where the agent is: 6-Hexyl-7-Hydroxy-3-[5-(4-Methylphenyl)-1,3,4-Oxadiazol-2-Yl]-2h-Chromen-2-One; 3-(6,7-Dimethoxy-1-{[(2-Methoxyphenyl)Carbamoyl]Methyl}-2,4-Dioxo-1,2,3,4-Tetrahydroquinazolin-3-Yl)-N-(2-Methoxyethyl)Propanamide; 1-(2-Ethylphenyl)-4-[1-(4-Phenoxybutyl)-1h-1,3-Benzodiazol-2-Yl]Pyrrolidin-2-One; 3-[2-(Azepan-1-Yl)-2-Oxoethyl]-5-[(3-Chlorophenyl)Methyl]-3h,4h,5h-Pyridazino[4,5-B]Indol-4-One; 3-[(3-Chloro-4-Methoxyphenyl)Amino]-2-Phenyl-1h-Inden-1-One; 2-[(1e)-2-[4-(Dimethylamino)Phenyl]Ethenyl]-3-(2-Methylphenyl)-3,4-Dihydroquinazolin-4-One; N-{3-[Butyl(Methyl)Amino]Propyl}-2-{5-[(3-Chlorophenyl)Methyl]-4-Oxo-3h,4h,5h-Pyridazino[4,5-B]Indol-3-Yl}Acetamide; and/or 2-Benzoyl-6-Ethyl-5h,6h, 7h,8 h-Thieno[2,3-B]1,6-Naphthyridin-3-Amine. The kit also includes directions for administering the agent to treat a bacterial infection.

In any of the above aspects, or embodiments thereof, the agent inhibits the invasion of, uptake of, and/or intracellular survival of the bacteria.

In any of the above aspects, or embodiments thereof, the bacteria is a Burkholderia, Pseudomonas, or Mycobacterium.

In any of the above aspects, or embodiments thereof, the two-component system includes a histidine kinase. In any of the above aspects, or embodiments thereof, the two-component system regulates microaerobiosis in the bacterial cell. In any of the above aspects, or embodiments thereof, the two-component system functions in a nitrogen fixation and/or a respiration pathway in the bacterial cell. In any of the above aspects, or embodiments thereof, the two-component system is a FixLJ system.

In any of the above aspects, or embodiments thereof, the bacteria is a Burkholderia, Pseudomonas, or Mycobacterium.

In any of the above aspects, or embodiments thereof, the method reduces macrophage invasion by the bacterium, macrophage uptake of the bacterium, and/or intracellular survival by the bacterium.

In any of the above aspects, or embodiments thereof, the bacterial cell is a member of the Burkholderia genus.

In any of the above aspects, or embodiments thereof, the bacterial cell is a member of the Pseudomonas or Mycobacterium genus.

In any of the above aspects, or embodiments thereof, the Burkholderia is B. dolosa, B. multivorans, B. thailandensis, or B. cenocepacia.

In any of the above aspects, or embodiments thereof, the method reduces the virulence of the Burkholderia bacterium.

In any of the above aspects, or embodiments thereof, the method reduces the ability of the Burkholderia bacterium to invade an immune cell, reduces uptake of the Burkholderia bacterium by an immune cell, and/or reduces survival of the Burkholderia bacterium within an immune cell.

In any of the above aspects, or embodiments thereof, the immune cell is a macrophage.

In any of the above aspects, or embodiments thereof, the subject has cystic fibrosis or chronic granulomatous disease.

In any of the above aspects, or embodiments thereof, the agent is 6-HEXYL-7-HYDROXY-3-[5-(4-METHYLPHENYL)-1,3,4-OXADIAZOL-2-YL]-2H-CHROMEN-2-ONE.

Compositions and articles defined by the disclosure were isolated or otherwise manufactured in connection with the examples provided below. Other features and advantages of the disclosure will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a polypeptide, nucleic acid molecule, or small molecule. In embodiments, the agent is an antimicrobial (e.g., antibiotic). In embodiments, the agent is a small molecule that reduces or inhibits the virulence of a bacteria (e.g., a member of the Burkholderia, Pseudomonas, or Mycobacterium genus). In particular embodiments, the agent is a small molecule shown in FIG. 1 (BFA-1-BFA-8) or listed in Table 5.

By “ameliorate” is meant decrease, suppress, attenuate, diminish, arrest, or stabilize the development or progression of a disease. In some embodiments, the disease is a bacterial infection. In some embodiments, the disease is a bacteria infection associated with a member of the Burkholderia, Pseudomonas, or Mycobacterium genus.

By “alteration” is meant a change (e.g., increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels. In some embodiments, the alteration is a change in the expression levels or activity of FixL, FixJ, or other members of the FixLJ pathway.

By “analog” is meant a molecule that is not identical, but has analogous functional or structural features. For example, a small molecule analog retains the biological activity of a corresponding small molecule, while having certain biochemical modifications that enhance the analog's function relative to the original small molecule. Such biochemical modifications could increase the analog's half-life, alter the analog's release profile, or increase the analog's bioavailability, for example, without altering, for example, ligand binding.

By “antimicrobial” is meant an agent that inhibits the growth of a pathogen and/or inhibits or reduces the virulence of a pathogen. In an embodiment, the antimicrobial is an agent that inhibits or reduces the virulence of a member of the Burkholderia, Pseudomonas, or Mycobacterium genus.

By “FixL polypeptide” is meant a protein or fragment thereof having at least 85% amino acid sequence identity to any one of amino acid sequences associated with GenBank Accession Nos. AJY12582.1, WP_045552426.1, KHS13643.1, BEH37199.1, WFN17399.1, WP_011694325.1, WP_059970324.1, WP_047898502.1, WP_040127770.1, WP_059678730.1, WP_059233707.1, WP_301820165.1, WP_301763846.1, WP_301748163.1, WP_026044610.1, WP 263074742.1, UXZ89649.1, or QRA12270.1 and having oxygen sensing and/or histidine kinase activity. Exemplary FixL polypeptide sequences are provided below:

>AJY12582.1 sensory box protein [Burkholderia dolosa AU0158]
(SEQ ID NO: 1)
MLTDRLFARSARPSGPPAESQPSRWHHGPWWSNSYLLTPLLSILVFLVVMSLILWSLNRREQQQQEDTLF
RNVAWAQQQIRLSMTGAQEQLQALSRDLASGRLDQNAFQMAVADVMQTHPEILYLNWYTAPGVQRWPSVH
PPLLGQRLAKPGDAQLQEAVRGAYDEARSTRRQAYSPLIYDDFGNGFITLQTPVMRGDREYLGSIAAVFS
VEGILKHDIPQELSSKYKISITDANNRELSSTSTRPRLPRDSHYDLPLDPPGQGLTVRVYAFPQLTNLTN
NTLVWLVAGLSCFVLWSLWSLWKHTRQRFEAQQALYAEAFFRRAMENSVLIGMRVLDMHGRITHVNPAFC
RMTGWDESDLVGKVAPFPYWPRDAYPEMQRQLDMTLRGKAPSSGFELRVRRKNGTLFHARLYVSPLIDSS
GRQTGSMSSMTDITEPKRAREELAAAHERFTTVLESLDAAVSVLAADEAELLFANRYYRHLFGIRPDGHL
ELSGGGFDRAQASSDSIDMVDAFAGLPAAALTNSTADAQEVYVESIQKWFEVRRQYIQWVDGHLAQMQIA
TDITTRKKAQELAHQQEEKLQFTSRLMTMGEMASSIAHELNQPLAAINNYCSGTLALVKSGRATPETLQP
ALEKTAQQALRAGMIVKRIREFVKRSEPKRQPARVADIVADAVGLAEIEARKRRIRIVTEILARMPIIYV
DPVLIEQVLVNLMKNAAEAMADVKPASADGVIRVVADIDAGFVDIRVIDQGPGVDEATAERLFEPFYSTK
SDGMGMGLNICRSIIESHRGRLWVVNNVEPDGRISGATFHCSLPIGEPADLGRGGSEASASHTVTGEL
>WP_045552426.1 oxygen sensor histidine kinase FixL [Burkholderia dolosa]
(SEQ ID NO: 1)
MLTDRLFARSARPSGPPAESQPSRWHHGPWWSNSYLLTPLLSILVFLVVMSLILWSLNRREQQQQEDTLF
RNVAWAQQQIRLSMTGAQEQLQALSRDLASGRLDQNAFQMAVADVMQTHPEILYLNWYTAPGVQRWPSVH
PPLLGQRLAKPGDAQLQEAVRGAYDEARSTRRQAYSPLIYDDFGNGFITLQTPVMRGDREYLGSIAAVFS
VEGILKHDIPQELSSKYKISITDANNRELSSTSTRPRLPRDSHYDLPLDPPGQGLTVRVYAFPQLTNLTN
NTLVWLVAGLSCFVLWSLWSLWKHTRQRFEAQQALYAEAFFRRAMENSVLIGMRVLDMHGRITHVNPAFC
RMTGWDESDLVGKVAPFPYWPRDAYPEMQRQLDMTLRGKAPSSGFELRVRRKNGTLFHARLYVSPLIDSS
GRQTGSMSSMTDITEPKRAREELAAAHERFTTVLESLDAAVSVLAADEAELLFANRYYRHLFGIRPDGHL
ELSGGGFDRAQASSDSIDMVDAFAGLPAAALTNSTADAQEVYVESIQKWFEVRRQYIQWVDGHLAQMQIA
TDITTRKKAQELAHQQEEKLQFTSRLMTMGEMASSIAHELNQPLAAINNYCSGTLALVKSGRATPETLQP
ALEKTAQQALRAGMIVKRIREFVKRSEPKRQPARVADIVADAVGLAEIEARKRRIRIVTEILARMPIIYV
DPVLIEQVLVNLMKNAAEAMADVKPASADGVIRVVADIDAGFVDIRVIDQGPGVDEATAERLFEPFYSTK
SDGMGMGLNICRSIIESHRGRLWVVNNVEPDGRISGATFHCSLPIGEPADLGRGGSEASASHTVTGEL
>KHS13643. 1 ATPase [Burkholderia multivorans]
(SEQ ID NO: 2)
MLTDRLFARSARPSGPPAESQPSRWHHGPWWSNSYLLTPLLSILVFLVVMSLILWSLNRREQQQQEDTLF
RNVAWAQQQIRLSMTGAQEQLQALSRDLASGRLDQNAFQMAVADVMQTHPEILYLNWYTAPGVQRWPTVH
PPLLGQRLAKPGDAQLQDAVRGAYDEARSSRRQAYSPLIYDDFGNGYITLQTPVMRGDRDYLGSLAAVFS
VEGILKHDIPQELSSKYKISITDANNRELSSTSTRPRLPRDSHYDLPLDPPGQGLTVRVYAFPQLTNLTN
NTLVWLVAGLSCFVLWSLWSLWKHTRQRFEAQQALYAEAFFRRAMENSVLIGMRVLDMHGRITHVNPAFC
RMMGWDESDLVGKVAPFPYWPRDAYPEMQRQLDMTLRGKAPSSGFELRVRRKNGTLFHARLYVSPLIDSS
GRQTGWMSSMTDITEPKRAREELAAAHERFTTVLESLDAAVSVLAADEAELLFANRYYRHLFGIRPDGHL
ELSGGGFDRAQASSDSIDMVDAFAGLPATALTNTAADAQEVYVESIQKWFEVRRQYIQWVDGHLAQMQIA
TDITTRKKAQELAHQQEEKLQFTSRLMTMGEMASSIAHELNQPLAAINNYCSGTLALVKSGRGTPEMLQP
ALEKTAQQALRAGMIVKRIREFVKRSEPKRQPARVADIVADAVGLAEIEARKRRIRIVTEILARMPIIYV
DPVLIEQVLVNLMKNAAEAMADVKPPSADGVIRVVADIDAGFVDIRVIDQGPGVDEATAERLFEPFYSTK
SDGMGMGLNICRSIIESHRGRLWVVNNVEPDGRISGATFHCSLPIGEPADLGQGGSETSASHTVTGEL
>BEH37199.1 oxygen sensor histidine kinase FixL [Burkholderia pseudomallei]
(SEQ ID NO: 3)
MQDALSYNPAMLTDRLFARSARPSGSPAESQPSRWHHGPWWSNSYLLTPLLSILVFLVVMSLILWSLNRR
EEQQQEDTLFRNVAWAQQQIRLSMTSAQEQLQAFSRDIAAGRIDEHAFQATVGDVMQAHPEILYLNWYTS
PGTKRWPTMQLPLLGQRLAKPNDAQMDEVVRGAYAQARGTRRQSYSPLVYDDFGNGYLTLQTPVIREREY
LGSIAAVFSVEGILKHDIPPELSAKYKISITDANNRELASTSSRPRLPRDAHYDLPLDPPGQGLTVRVYA
YPQTTNLTNNTLVWLVAGLSCFVLWSLWSLWKHTRQRFEAQQALYAEAFFRRAMENSVLIGMRVLDMHGR
ITHVNPAFCRMTGWDESDLVGKTAPFPYWPRDAYPEMQRQLDMTLRGKAPSSGFELRVRRKDGSLFHARL
YVSPLIDSAGRQTGWMSSMTDITEHKRAREELAAAHERFTTVLESLDAAVSVLAADEAELLFANRYYRHL
FGIRPDGHLELSGGGFDTAQASSDSIDMVDAYAGLPAAALTESTADAQEVYVESIQKWFEVRRQYIQWVD
GHLAQMQIATDITTRKKAQELAHQQEEKLQFTSRLMTMGEMASSIAHELNQPLAAINNYCSGTLALVKSG
RASPETLAPALEKTAQQALRAGMIVKRIREFVKRSEPKRQPSRVADIVADAVGLAEIEARKRRIRIVTEI
RARMPIIYVDPVLIEQVLVNLMKNAAEAMQEARPQAENGVIRVVADLEAGFVDIRVIDQGPGVDEATAER
LFEPFYSTKSDGMGMGLNICRSIIESHRGRLWVVNNVEPDGLVSGATFHCSLPIGEPEDLGRGSETSPSQ
TVTGEI
>WFN17399.1 oxygen sensor histidine kinase FixL [Burkholderia contaminans]
(SEQ ID NO: 4)
MLTDRLFARSARPSGPPAESQPSRWHHGPWWSNSYLLTPLLSILVFLVVMSLILWSLNRREQQQQEDTLF
RNVAWAQQQIRLSMTGAQEQLQAVSRDLASGRLDQNAFQMAVADVMQTHPEILYLNWYTSPGVQRWPTVH
PPLLGQRLAKPGDAQMQDAVRGAYDEARSTRRQAYSPLIYDDFGNGFITLQTPVMRGDREYLGSIAAVFS
VEGILKHDIPQELSSKYKISITDANNRELSSTSTRPRLPRDSHYDLPLDPPGQGLTVRVYAFPQLTNLTN
NTLVWLVAGLSCFVLWSLWSLWKHTRQRFEAQQALYAEAFFRRAMENSVLIGMRVLDMHGRITHVNPAFC
RMTGWDETDLVGKVAPFPYWPRDAYPEMQRQLDMTLRGKAPSSGFELRVRRKNGTLFHARLYVSPLIDSS
GRQTGWMSSMTDITEPKRAREELAAAHERFTTVLESLDAAVSVLAADEAELLFANRYYRHLFGIRPDGHL
ELSGGGFDRAQASSDSIDMVDAFAGLPAAALTSSTADAQEVYVESIQKWFEVRRQYIQWVDGHLAQMQIA
TDITTRKKAQELAHQQEEKLQFTSRLMTMGEMASSIAHELNQPLAAINNYCSGTLALVKSGRGTPETLQP
ALEKTAQQALRAGMIVKRIREFVKRSEPKRQPARVADIVADAVGLAEIEARKRRIRIVTEILARMPIIYV
DPVLIEQVLVNLMKNAAEAMADVKPASVDGVIRVVADIEAGFVDIRVIDQGPGVDEATAERLFEPFYSTK
SDGMGMGLNICRSIIESHRGRLWVVNNVEPDGRISGATFHCSLPIGEPADLGQGGREASASHTVTGEL
>WP_011694325.1 MULTISPECIES: oxygen sensor histidine kinase FixL
[Burkholderia cepacia complex]
(SEQ ID NO: 5)
MLTDRLFARSARPPGPPAGSQPSRWHHGPWWSNSYLLTPLLSILVFLVVMSLILWSLNRREQQQQEDTLF
RNVAWAQQQIRLSMTGAQEQLQALSRDLASGRLDQNAFQMAVADVMQTHPEILYLNWYTAPGVQRWPTVH
PPLLGQRLAKPGDAQMQDAVRGAYDEARSTRRQAYSPLIYDDFGNGFITLQTPVMRGDREYLGSIAAVES
VEGILKHDIPQELSSKYKISITDSNNRELSSTSTRPRLPRDSHYDLPLDPPGQGLTVRVYAFPQLTNLTN
NTLVWLVAGLSCFVLWSLWSLWKHTRQRFEAQQALYAEAFFRRAMENSVLIGMRVLDMHGRITHVNPAFC
RMTGWDETDLVGKVAPFPYWPRDAYPEMQRQLDMTLRGKAPSSGFELRVRRKNGTLFHARLYVSPLIDSS
GRQTGWMSSMTDITEPKRAREELAAAHERFTTVLESLDAAVSVLAADEAELLFANRYYRHLFGIRPDGHL
ELSGGGFDRAQASSDSIDMVDAFAGLPAAALTSSTADAQEVYVESIQKWFEVRRQYIQWVDGHLAQMQIA
TDITTRKKAQELAHQQEEKLQFTSRLMTMGEMASSIAHELNQPLAAINNYCSGTLALVKSGRGTPETLQP
ALEKTAQQALRAGMIVKRIREFVKRSEPKRQPARVADIVADAVGLAEIEARKRRIRIVTEILARMPIIYV
DPVLIEQVLMNLMKNAAEAMADVKPASADGVIRVVADIDAGFVDIRVIDQGPGVDEATAERLFEPFYSTK
SDGMGMGLNICRSIIESHRGRLWVVNNVEPDGRISGATFHCSLPIGEPADLGQGGREASASHTVTGEL
>WP_059970324.1 MULTISPECIES : oxygen sensor histidine kinase FixL
[Burkholderia cepacia complex]
(SEQ ID NO: 6)
MLTDRLFARSARPSGPPAESQPSRWHHGPWWSNSYLLTPLLSILVFLVVMSLILWSLNRREQQQQEDTLF
RNVAWAQQQIRLSMTGAQEQLQALSRDLASGRLDQNGFQMAVADVMQTHPEILYLNWYTSPGTQRWPTVH
PPLLGQRLARPGDAQMQDAVRGAYDEARNSRRQAYAPLVYDDFGNGYITLQTPVMRGDREYLGSLAAVFS
VEGILKHDIPQELSSKYKISITDANNRELSSTSSRPRLPRDSHYDLPLDPPGQGLTVRVYAFPQLTNLTN
NTLVWLVAGLSCFVLWSLWSLWKHTRQRFEAQQALYAEAFFRRAMENSVLIGMRVLDMHGRITHVNPAFC
RMTGWDESDLVGKVAPFPYWPRDSYPEMQRQLDMTLRGKAPSSGFELRVRRKNGTLFHARLYVSPLIDSA
GRQTGWMSSMTDITEPKRAREELAAAHERFTTVLESLDAAVSVLAADEAELLFANRYYRHLFGIRPDGHL
ELSGGGFDRAQASSDSIDMVDTFAGLPAAALTGSTADAQEVYVESIQKWFEVRRQYIQWVDGHLAQMQIA
TDITTRKKAQELAHQQEEKLQFTSRLMTMGEMASSIAHELNQPLAAINNYCSGTLAMVKSGRATTETLAP
ALEKTAQQALRAGMIVKRIREFVKRSEPKRQPARVADIVADAVGLAEIEARKRRIRIVTEILARMPIIYV
DPVLIEQVLVNLMKNAAEAMADVKPASADGVIRVIADLEAGFVDIRVIDQGPGVDEATAERLFEPFYSTK
SDGMGMGLNICRSIIESHRGRLWVVNNVEADGRISGATFHCSLPIGEPADLARGGAEASASHTVTGEL
>WP_047898502. 1 MULTISPECIES: oxygen sensor histidine kinase FixL
[Burkholderia cepacia complex]
(SEQ ID NO: 7)
MLTDRLFARSARPSGPPAESQPSRWHHGPWWSNSYLLTPLLSILVFLVVMSLILWSLNRREQQQQEDTLF
RNVAWAQQQIRLSMTGAQEQLQALSRDLASGRLDQNAFQMAVADVMQTHPEILYLNWYTAPGVQRWPTVH
PPLLGQRLAKPGDAQMQDAVRGAYDEARSTRRQAYSPLIYDDFGNGFITLQTPVMRGDREYLGSIAAVFS
VEGILKHDIPQELSSKYKISITDANNRELSSTSTRPRLPRDSHYDLPLDPPGQGLTVRVYAFPQLTNLTN
NTLVWLVAGLSCFVLWSLWSLWKHTRQRFEAQQALYAEAFFRRAMENSVLIGMRVLDMHGRITHVNPAFC
RMTGWDESDLVGKVAPFPYWPRDAYPEMQRQLDMTLRGKAPSSGFELRVRRKNGTLFHARLYVSPLIDSS
GRQTGWMSSMTDITEPKRAREELAAAHERFTTVLESLDAAVSVLAADEAELLFANRYYRHLFGIRPDGHL
ELSGGGFDRAQASSDSIDMVDAFAGLPAAALTSSTADAQEVYVESIQKWFEVRRQYIQWVDGHLAQMQIA
TDITTRKKAQELAHQQEEKLQFTSRLMTMGEMASSIAHELNQPLAAINNYCSGTLALVKSGRGTPETLQP
ALEKTAQQALRAGMIVKRIREFVKRSEPKRQPARVADIVADAVGLAEIEARKRRIRIVTEILARMPIIYV
DPVLIEQVLMNLMKNAAEAMADVKPASADGVIRVVADIDAGFVDIRVIDQGPGVDEATAERLFEPFYSTK
SDGMGMGLNICRSIIESHRGRLWVVNNVEPDGRISGATFHCSLPIGEPADLGQGGREASASHTVTGEL
>WP_040127770. 1 MULTISPECIES: oxygen sensor histidine kinase FixL
[Burkholderia cepacia complex]
(SEQ ID NO: 8)
MLTDRLFARSARPSGPPAESQPSRWHHGPWWSNSYLLTPLLSILVFLVVMSLILWSLNRREQQQQEDTLF
RNVAWAQQQIRLSMTGAQEQLQALSRDLASGRLDQNAFQMAVADVMQTHPEILYLNWYTAPGVQRWPTVH
PPLLGQRLAKPGDAQLQDAVRGAYDEARSSRRQAYSPLIYDDFGNGYITLQTPVMRGDRDYLGSIAAVFS
VEGILKHDIPQELSSKYKISITDANNRELSSTSTRPRLPRDSHYDLPLDPPGQGLTVRVYAFPQLTNLTN
NTLVWLVAGLSCFVLWSLWSLWKHTRQRFEAQQALYAEAFFRRAMENSVLIGMRVLDMHGRITHVNPAFC
RMTGWDETDLVGKVAPFPYWPRDAYPEMQRQLDMTLRGKAPSSGFELRVRRKNGTLFHARLYVSPLIDSS
GRQTGWMSSMTDITEPKRAREELAAAHERFTTVLESLDAAVSVLAADEAELLFANRYYRHLFGIRPDGHL
ELSGGGFDRAQASSDSIDMVDAFAGLPAAALTSSAADAQEVYVESIQKWFEVRRQYIQWVDGHLAQMQIA
TDITTRKKAQELAHQQEEKLQFTSRLMTMGEMASSIAHELNQPLAAINNYCSGTLALVKSGRGTPETLQP
ALEKTAQQALRAGMIVKRIREFVKRSEPKRQPARVADIVADAVGLAEIEARKRRIRIVTEILARMPIIYV
DPVLIEQVLVNLMKNAAEAMADVKPASADGVIRVVADIDAGFVDIRVIDQGPGVDEATAERLFEPFYSTK
SDGMGMGLNICRSIIESHRGRLWVVNNVEPDGRISGATFHCSLPIGEPADLGQGGREASASHTVTGEL
>WP_059678730. 1 MULTISPECIES: oxygen sensor histidine kinase FixL
[Burkholderia]
(SEQ ID NO: 9)
MLTDRLFARSARPSGPPAESQPSRWHHGPWWSNSYLLTPLLSILVFLVVMSLILWSLNRREQQQQEDTLF
RNVAWAQQQIRLSMTGAQEQLQALSRDLASGRLDQNAFQMAVADVMQTHPEILYLNWYTAPGVQRWPTVH
PPLLGQRLAKPGDAQMQDAVRGAYDEARTTRRQAYSPLIYDDFGNGFITLQTPVMRGDREYLGSIAAVFS
VEGILKHDIPQELSSKYKISITDSNNRELSSTSTRPRLPRDSHYDLPLDPPGQGLTVRVYAFPQLTNLTN
NTLVWLVAGLSCFVLWSLWSLWKHTRQRFEAQQALYAEAFFRRAMENSVLIGMRVLDMHGRITHVNPAFC
RMTGWDESDLVGKVAPFPYWPRDAYPEMQRQLDMTLRGKAPSSGFELRVRRKNGTLFHARLYVSPLIDSS
GRQTGWMSSMTDITEPKRAREELAAAHERFTTVLESLDAAVSVLAADEAELLFANRYYRHLFGIRPDGHL
ELSGGGFDRAQASSDSIDMVDAFAGLPAAALTSSTADAQEVYVESIQKWFEVRRQYIQWVDGHLAQMQIA
TDITTRKKAQELAHQQEEKLQFTSRLMTMGEMASSIAHELNQPLAAINNYCSGTLALVKSGRGTPETLQP
ALEKTAQQALRAGMIVKRIREFVKRSEPKRQPARVADIVADAVGLAEIEARKRRIRIVTEILARMPIIYV
DPVLIEQVLMNLMKNAAEAMADVKPASADGVIRVVADIDAGFVDIRVIDQGPGVDEATAERLFEPFYSTK
SDGMGMGLNICRSIIESHRGRLWVVNNVEPDGRISGATFHCSLPIGEPADLGQGGREASASHTVTGEL
>WP_059233707.1 oxygen sensor histidine kinase FixL [Burkholderia arboris]
(SEQ ID NO: 10)
MLTDRLFARSARPPGPPAESQPSRWHHGPWWSNSYLLTPLLSILVFLVVMSLILWSLNRREQQQQEDTLF
RNVAWAQQQIRLSMTGAQEQLQALSRDLASGRLDQNAFQMAVADVMQTHPEILYLNWYTAPGVQRWPTVH
PPLLGQRLAKPGDAQMQDAVRGAYDEARSTRRQAYSPLIYDDFGNGFITLQTPVMRGDREYLGSIAAVFS
VEGILKHDIPQELSSKYKISITDANNRELSSTSTRPRLPRDSHYDLPLDPPGQGLTVRVYAFPQLTNLTN
NTLVWLVAGLSCFVLWSLWSLWKHTRQRFEAQQALYAEAFFRRAMENSVLIGMRVLDMHGRITHVNPAFC
RMTGWDETDLVGKVAPFPYWPRDAYPEMQRQLDMTLRGKAPSSGFELRVRRKNGTLFHARLYVSPLIDSS
GRQTGWMSSMTDITEPKRAREELAAAHERFTTVLESLDAAVSVLAADEAELLFANRYYRHLFGIRPDGHL
ELSGGGFDRAQASSDSIDMVDAFAGLPAAALTSSTADAQEVYVESIQKWFEVRRQYIQWVDGHLAQMQIA
TDITTRKKAQELAHQQEEKLQFTSRLMTMGEMASSIAHELNQPLAAINNYCSGTLALVKSGRGTPETLQP
ALEKTAQQALRAGMIVKRIREFVKRSEPKRQPARVADIVADAVGLAEIEARKRRIRIVTEILARMPIIYV
DPVLIEQVLMNLMKNAAEAMADVKPAAADGVIRVVADIDAGFVDIRVIDQGPGVDEATAERLFEPFYSTK
SDGMGMGLNICRSIIESHRGRLWVVNNVEPDGRISGATFHCSLPIGEPADLGQGGREASASHTVTGEL
>WP_301820165.1 oxygen sensor histidine kinase FixL [Burkholderia
vietnamiensis]
(SEQ ID NO: 11)
MLTDRLFARSARPSGPPAESQPSRWHHGPWWSNSYLLTPLLSILVFLVVMSLILWSLNRREQQQQEDTLF
RNVAWAQQQIRLSMTGAQEQLQALSRDLASGRLDQNAFQMAVADVMQTHPEILYLNWYTAPGVQRWPTVH
PPLLGQRLAKPGDAQMEDAVRGAYDEARSTRRQAYSPLIYDDFGNGFITLQTPVMRGDREYLGSIAAVFS
VEGILKHDIPQELSSKYKISITDSNNRELSSTSTRPRLPRDSHYDLPLDPPGQGLTVRVYAFPQLTNLTN
NTLVWLVAGLSCFVLWSLWSLWKHTRQRFEAQQALYAEAFFRRAMENSVLIGMRVLDMHGRITHVNPAFC
RMTGWDESDLVGKVAPFPYWPRDAYPEMQRQLDMTLRGKAPSSGFELRVRRKNGTLFHARLYVSPLIDSS
GRQTGWMSSMTDITEPKRAREELAAAHERFTTVLESLDAAVSVLAADEAELLFANRYYRHLFGIRPDGHL
ELSGGGFDRAQASSDSIDMVDAFAGLPAAALTNSTADAQEVYVESIQKWFEVRRQYIQWVDGHLAQMQIA
TDITTRKKAQELAHQQEEKLQFTSRLMTMGEMASSIAHELNQPLAAINNYCSGTLALVKSGRSTPETLQP
ALEKTAQQALRAGMIVKRIREFVKRSEPKRQPARVADIVADAVGLAEIEARKRRIRIVTEILARMPIIYV
DPVLIEQVLVNLMKNAAEAMADVKAASADGVIRVVADIDAGFVDIRVIDQGPGVDEATAERLFEPFYSTK
SDGMGMGLNICRSIIESHRGRLWVVNNVEPDGRISGATFHCSLPIGEPADLAQGGRESSASHTVTGEL
>WP_301763846.1 oxygen sensor histidine kinase FixL [Burkholderia cepacia]
(SEQ ID NO: 12)
MLTDRLFARSARPSGPPAESQPSRWHHGPWWSNSYLLTPLLSILVFLVVMSLILWSLNRREQQQQEDTLF
RNVAWAQQQIRLSMTGAQEQLQALSRDLASGRLDQNAFQMAVADVMQTHPEILYLNWYTSPGVQRWPTVH
PPLLGQRLAKPGEAQMQDAVRGAYDEARSTRRQAYSPLIYDDFGNGFITLQTPVMRGDREYLGSIAAVFS
VEGILKHDIPQELSSKYKISITDANNRELSSTSTRPRLPRDSHYDLPLDPPGQGLTVRVYAFPQLTNLTN
NTLVWLVAGLSCFVLWSLWSLWKHTRQRFEAQQALYAEAFFRRAMENSVLIGMRVLDMHGRITHVNPAFC
RMTGWDETDLVGKVAPFPYWPRDAYPEMQRQLDMTLRGKAPSSGFELRVRRKNGTLFHARLYVSPLIDSS
GRQTGWMSSMTDITEPKRAREELAAAHERFTTVLESLDAAVSVLAADEAELLFANRYYRHLFGIRPDGHL
ELSGGGFDRAQASSDSIDMVDAFAGLPAAALTSSTADAQEVYVESIQKWFEVRRQYIQWVDGHLAQMQIA
TDITTRKKAQELAHQQEEKLQFTSRLMTMGEMASSIAHELNQPLAAINNYCSGTLALVKSGRGTPETLQP
ALEKTAQQALRAGMIVKRIREFVKRSEPKRQPARVADIVADAVGLAEIEARKRRIRIVTEILARMPIIYV
DPVLIEQVLVNLMKNAAEAMADVKPALADGVIRVIADIEAGFVDIRVIDQGPGVDEATAERLFEPFYSTK
SDGMGMGLNICRSIIESHRGRLWVVNNVEPDGRISGATFHCSLPIGEPADLGQGGREASASHTVTGEL
>WP_301748163.1 oxygen sensor histidine kinase FixL [Burkholderia orbicola]
(SEQ ID NO: 13)
MLTDRLFARSARPPGPPAGSQPSRWHHGPWWSNSYLLTPLLSILVFLVVMSLILWSLNRREQQQEDTLER
NVAWAQQQIRLSMTGAQEQLQALSRDLASGRLDQNAFQMAVADVMQTHPEILYLNWYTAPGVQRWPTVHP
PLLGQRLAKPGDAQMQDAVRGAYDEARSTRRQAYSPLIYDDFGNGFITLQTPVMRGDREYLGSIAAVFSV
EGILKHDIPQELSSKYKISITDSNNRELSSTSTRPRLPRDSHYDLPLDPPGQGLTVRVYAFPQLTNLTNN
TLVWLVAGLSCFVLWSLWSLWKHTRQRFEAQQALYAEAFFRRAMENSVLIGMRVLDMHGRITHVNPAFCR
MTGWDETDLVGKVAPFPYWPRDAYPEMQRQLDMTLRGKAPSSGFELRVRRKNGTLFHARLYVSPLIDSSG
RQTGWMSSMTDITEPKRAREELAAAHERFTTVLESLDAAVSVLAADEAELLFANRYYRHLFGIRPDGHLE
LSGGGFDRAQASSDSIDMVDAFAGLPAAALTSSTADAQEVYVESIQKWFEVRRQYIQWVDGHLAQMQIAT
DITTRKKAQELAHQQEEKLQFTSRLMTMGEMASSIAHELNQPLAAINNYCSGTLALVKSGRGTPETLQPA
LEKTAQQALRAGMIVKRIREFVKRSEPKRQPARVADIVADAVGLAEIEARKRRIRIVTEILARMPIIYVD
PMLIEQVLMNLMKNAAEAMADVKPASADGVIRVVADIDAGFVDIRVIDQGPGVDEATAERLFEPFYSTKS
DGMGMGLNICRSIIESHRGRLWVVNNVEPDGRISGATFHCSLPIGEPADLGQGGREASASHTVTGEL
>WP_026044610.1 oxygen sensor histidine kinase FixL [Burkholderia pyrrocinia]
(SEQ ID NO: 14)
MLTDRLFARSARPSGPPAESQPSRWHHGPWWSNSYLLTPLLSILVFLVVMSLILWSLNRREQQQQEDTLF
RNVAWAQQQIRLSMTGAQEQLQALSRDLASGRLDQNGFQMAVADVMQTHPEILYLNWYTSPGTQRWPTVH
PPLLGQRLARPGDAQMQDAVRGAYDEARNSRRQAYAPLVYDDFGNGYITLQTPVMRGDREYLGSLAAVFS
VEGILKHDIPQELSSKYKISITDANNRELSSTSSRPRLPRDSHYDLPLDPPGQGLTVRVYAFPQLTNLTN
NTLVWLVAGLSCFVLWSLWSLWKHTRQRFEAQQALYAEAFFRRAMENSVLIGMRVLDMHGRITHVNPAFC
RMTGWDESDLVGKVAPFPYWPRDSYPEMQRQLDMTLRGKAPSSGFELRVRRKNGTLFHARLYVSPLIDSA
GRQTGWMSSMTDITEPKRAREELAAAHERFTTVLESLDAAVSVLAADEAELLFANRYYRHLFGIRPDGHL
ELSGGGFDRAQASSDSIDMVDTFAGLPAAALTGSTADAQEVYVESIQKWFEVRRQYIQWVDGHLAQMQIA
TDITTRKKAQELAHQQEEKLQFTSRLMTMGEMASSIAHELNQPLAAINNYCSGTLAMVKSGRATTETLAP
ALEKTAQQALRAGMIVKRIREFVKRSEPKRQPARVADIVADAVGLAEIEARKRRIRIVTEILARMPIIYV
DPVLIEQVLVNLMKNAAEAMADVKPASADGVIRVIANLEAGFVDIRVIDQGPGVDEATAERLFEPFYSTK
SDGMGMGLNICRSIIESHRGRLWVVNNVEADGRISGATFHCSLPIGEPADLARGGAEASASHTVTGEL
>WP_263074742.1 oxygen sensor histidine kinase FixL [Burkholderia
multivorans]
(SEQ ID NO: 15)
MLTDRLFARSARPSGPPAESQPSRWHHGPWWSNSYLLTPLLSILVFLVVMSLILWSLNRREQQQQEDTLF
RNVAWAQQQIRLSMTGAQEQLQALSRDLASGRLDQNAFQMAVADVMQTHPEILYLNWYTAPGVQRWPTVH
PPLLGQRLAKPGDAQLQDAVRGAYDEARSSRRQAYSPLIYDDFGNGYITLQTPVMRGDRDYLGSLAAVFS
VEGILKHDIPQELSSKYKISITDANNRELSSTSTRPRLPRDSHYDLPLDPPGQGLTVRVYAFPQLTNLTN
NTLVWLVAGLSCFVLWSLWSLWKHTRQRFEAQQALYAEAFFRRAMENSVLIGMRVLDMHGRITHVNPAFC
RMTGWDESDLVGKVAPFPYWPRDAYPEMQRQLDMTLRGKAPSSGFELRVRRKNGTLFHARLYVSPLIDSS
GRQTGWMSSMTDITEPKRAREELAAAHERFTTVLESLDAAVSVLAADEAELLFANRYYHHLFGIRPDGHL
ELSGGGFDRAQASSDSIDMVDAFAGLPATALTNTAADAQEVYVESIQKWFEVRRQYIQWVDGHLAQMQIA
TDITTRKKAQELAHQQEEKLQFTSRLMTMGEMASSIAHELNQPLAAINNYCSGTLALVKSGRGTPEMLQP
ALEKTAQQALRAGMIVKRIREFVKRSEPKRQPARVADIVADAVGLAEIEARKRRIRIVTEILARMPIIYV
DPVLIEQVLVNLMKNAAEAMADVKPPSADGVIRVVADIDAGFVDIRVIDQGPGVDEATAERLFEPFYSTK
SDGMGMGLNICRSIIESHRGRLWVVNNVEPDGRISGATFHCSLPIGEPADLGQGGSETSASHTVTGEL
>UXZ89649.1 oxygen sensor histidine kinase FixL [Burkholderia cenocepacia]
(SEQ ID NO: 16)
MLTDRLFARSARPPGPPAESQPSRWHHGPWWSNSYLLTPLLSILVFLVVMSLILWSLNRREQQQQEDTLF
RNVAWAQQQIRLSMTGAQEQLQALSRDLASGRLDQNAFQMAVADVMQTHPEILYLNWYTAPGVQRWPTVH
PPLLGQRLAKPGDAQMQDAVRGAYDEARSTRRQAYSPLIYDDFGNGFITLQTPVMRGDRDYLGSIAAVFS
VEGILKHDIPQELSSKYKISITDSNNRELSSTSTRPRLPRDSHYDLPLDPPGQGLTVRVYAFPQLTNLTN
NTLVWLVAGLSCFVLWSLWSLWKHTRQRFEAQQALYAEAFFRRAMENSVLIGMRVLDMHGRITHVNPAFC
RMTGWDETDLVGKVAPFPYWPRDAYPEMQRQLDMTLRGKAPSSGFELRVRRKNGTLFHARLYVSPLIDSS
GRQTGWMSSMTDITEPKRAREELAAAHERFTTVLESLDAAVSVLAADEAELLFANRYYRHLFGIRPDGHL
ELSGGGFDRAQASSDSIDMVDAFAGLPAAALTSSTADAQEVYVESIQKWFEVRRQYIQWVDGHLAQMQIA
TDITTRKKAQELAHQQEEKLQFTSRLMTMGEMASSIAHELNQPLAAINNYCSGTLALVKSGRGTPETLQP
ALEKTAQQALRAGMIVKRIREFVKRSEPKRQPARVADIVADAVGLAEIEARKRRIRIVTEILARMPIIYV
DPVLIEQVLMNLMKNAAEAMADVKPASADGVIRVVADIDAGFVDIRVIDQGPGVDEATAERLFEPFYSTK
SDGMGMGLNICRSIIESHRGRLWVVNNVEPDGRISGATFHCSLPIGEPADLGQGGREASASHTVTGEL
>QRA12270.1 oxygen sensor histidine kinase FixL [Burkholderia thailandensis]
(SEQ ID NO: 17)
MLTDRLFARSARPSGSPAESQPSRWHHGPWWSNSYLLTPLLSILVFLVVMSLILWSLNRREEQQQEDTLF
RNVAWAQQQIRLSMTSAQEQLQAFSRDIAAGRIDEHAFQTTVGDVMQAHPEILYLNWYTSPGTKRWPTMQ
LPVLGQRLAKPGDAQMDEVVRGAYAQARSTRRQSYSPLVYDDFGNGYLTLQTPVIRDREYLGSIAAVFSV
EGILKHDIPPELSAKYKISITDANNRELASTSSRPRLPRDAHYDLPLDPPGQGLTVRVYAYPQTTNLTNN
TLVWLVAGLSCFVLWSLWSLWKHTRQRFEAQQALYAEAFFRRAMENSVLIGMRVLDMHGRITHVNPAFCR
MTGWDESDLVGKTAPFPYWPRDAYPEMQRQLDMTLRGKAPSSGFELRVRRKDGSLFHARLYVSPLIDSAG
RQTGWMSSMTDITEPKRAREELAAAHERFTTVLESLDAAVSVLAADEAELLFANRYYRHLFGIRPDGHLE
LSGGGFDTAQASSDSIDMVDAYAGLPAAALTESTADAQEVYVESIQKWFEVRRQYIQWVDGHLAQMQIAT
DITTRKKAQELAHQQEEKLQFTSRLMTMGEMASSIAHELNQPLAAINNYCSGTLALVKSGRASPETLAPA
LEKTAQQALRAGMIVKRIREFVKRSEPKRQPSRVADIVADAVGLAEIEARKRRIRIVTEIRARMPIIYVD
PVLIEQVLVNLMKNAAEAMQEARPQAENGVIRVVADLESGFVDIRVIDQGPGVDEATAERLFEPFYSTKS
DGMGMGLNICRSIIESHRGRLWVVNNVEPDGFVSGATFHCSLPIGEPEDLGHGSETSPSQTVTGEI

By “fixL polynucleotide” is meant a nucleic acid molecule encoding a FixL polypeptide. The sequence of an exemplary FixL polynucleotide follows:

>X56808.1 B. japonicum fixL and fixJ genes for FixL and FixJ proteins,
and ORF138 for nitrogen fixation
(SEQ ID NO: 18)
GCATGCCCGCCGCGATTCCTGGTGGCCGCGCGGAATTGCGCCTCCAGAGCCGCCAGCCGTCGCCTGACGG
ACGCTTCGCCCTCGTCGATCAGTCCCTGGGCGGCGGCGCCGTCGGTGAAATAGAGCGGCGGGGCGAACTG
CGCCGCGGCAACCCCGACGATGGCGGCCTCGAATCGCTCGGCGAGCTCGCCCGCGACCTGAAGACGCGCC
TCGTTGGACTGGTCGAGCGCCAAGCTGACCATCACGGTCGCGTATGTCATTAGAATTCTCCGGGCAATGA
ACTGCTATGAAATCTAGCTGTGCCAGCGCGAGGCAGGATGAGATAGATCAAACCGTCGCCCACCCGGGAC
CACCGGCATTACGCCAATGGAATCAAACATCCGAACTTGCCTCCGGCCCGGGCTTGAGCGAAATTACCGC
CGTGGTTGCGGCACCTGGGCCCGTGGCACGAGAATGGGAGTGACACCTTGGCGCCGACCCGCGTAACGCA
TCCGCCGGATGACGGCCGGGGCGAGCATTTCCGGGTCAGGATCGAGGGATTCGGCGTCGGCACCTGGGAT
CTCGACCTCAAGACCTGGGCGCTGGACTGGTCGGATACCGCGCGGACCCTGCTCGGAATCGGGCAGGACC
AGCCGGCGAGCTACGACCTCTTCCTGTCACGCCTCGAGCCCGACGACCGCGAGCGCGTGGAGAGCGCGAT
CAAGCGCGTCTCCGAACGCGGTGGCGGCTTCGACGTGTCCTTCAGGGTCGCCGGCACCTCCAACGCAGGA
CAGTGGATTCGCGCCCGGGCCGGACTCATTCGGGACGAAGCCGGCACCGCCCGCCATCTCAGCGGCATCT
TTCTCGATATCGACGAGGAGAAGCAGGTCGAGGGTGCGCTCCGTACCCGCGAGACCCACCTCCGCTCGAT
CCTCCACACAATTCCCGACGCCATGATCGTCATCGACGGCCACGGCATCATCCAGCTGTTCAGCACCGCC
GCCGAGCGCCTGTTCGGCTGGTCCGAGCTCGAGGCGATCGGCCAGAACGTCAACATCCTGATGCCGGAGC
CCGACCGCTCCCGGCATGACAGCTACATTTCGCGTTACCGCACCACGAGCGATCCCCACATCATCGGCAT
CGGGCGCATCGTGACCGGCAAGCGCCGCGACGGCACCACCTTCCCGATGCACCTGTCGATCGGCGAGATG
CAGTCCGGCGGCGAGCCCTATTTTACCGGTTTCGTCCGCGATCTCACCGAGCACCAGCAGACCCAGGCGC
GTCTCCAGGAACTGCAATCCGAGCTCGTCCACGTCTCCAGGCTGAGCGCCATGGGCGAAATGGCGTCCGC
GCTCGCGCACGAGCTCAACCAGCCGCTGGCGGCGATCAGCAACTACATGAAGGGCTCGCGGCGGCTGCTT
GCCGGCAGCAGTGATCCGAACACACCGAAGGTCGAAAGCGCCCTGGACCGCGCCGCCGAGCAGGCGCTGC
GCGCCGGCCAGATCATCCGGCGCCTGCGCGACTTCGTTGCCCGCGGCGAATCGGAGAAGCGGGTCGAGAG
TCTCTCCAAGCTGATCGAGGAGGCCGGCGCGCTCGGGCTTGCCGGCGCGCGCGAGCAGAACGTGCAGCTC
CGCTTCAGTCTCGATCCGGGCGCCGATCTCGTTCTCGCCGACCGGGTGCAGATCCAGCAGGTCCTGGTCA
ACCTGTTCCGCAACGCGCTGGAAGCGATGGCTCAGTCGCAGCGACGCGAGCTCGTCGTCACCAACACCCC
CGCCGCCGACGACATGATCGAGGTCGAAGTGTCCGACACCGGCAGCGGTTTCCAGGACGACGTCATTCCG
AACCTGTTTCAGACTTTCTTCACCACCAAGGACACCGGCATGGGCGTGGGACTGTCCATCAGCCGCTCGA
TCATCGAAGCTCACGGGGGGCGCATGTGGGCCGAGAGCAACGCATCGGGGGGGGCGACCTTCCGCTTCAC
CCTCCCGGCAGCCGACGAGAATTGATGCATGACGACCAAGGGACATATCTACGTCATCGACGACGACGCG
GCGATGCGGGATTCGCTGAATTTCCTGCTGGATTCTGCCGGCTTCGGCGTCACGCTGTTTGACGACGCGC
AAGCCTTTCTCGACGCCCTGCCGGGTCTCTCCTTCGGCTGCGTCGTCTCCGACGTGCGCATGCCGGGCCT
TGACGGCATCGAGCTGTTGAAGCGGATGAAGGCGCAGCAAAGCCCCTTTCCGATCCTCATCATGACCGGT
CACGGCGACGTGCCGCTCGCGGTCGAGGCGATGAAGTTAGGGGCGGTCGACTTTCTGGAAAAGCCTTTCG
AGGACGACCGCCTCACCGCCATGATCGAATCGGCGATCCGCCAGGCCGAGCCGGCCGCCAAGAGCGAGGC
CGTCGCGCAGGATATCGCCGCCCGCGTCGCCTCGTTGAGCCCCAGGGAGCGCCAGGTCATGGAAGGGCTG
ATCGCCGGCCTTTCCAACAAGCTGATCGCCCGCGAGTACGACATCAGCCCGCGCACCATCGAGGTGTATC
GGGCCAACGTCATGACCAAGATGCAGGCCAACAGCCTTTCGGAGCTGGTTCGCCTCGCGATGCGCGCCGG
CATGCTCAACGATTGAGCCAAGTCAAGGCCCTTGCCGGATGCTGTGCTAGCCATGCAGCATGATCGAGGT
CAGTTCACATCACGAGCGGCTGCCGTCCTCCACAAAACCCACCGTCTTTGTGGTCGATGATGACGCCGCA
GTCCTGGGATCCCTGCGGTTCCTGCTGGAAACCGACGGCTTTGCCGTGCGGACCTTCAGGAGCGGCACGG
CGCTGCTCAATGCCGGCGGCGCGCCCGGGGCCGACTGCTACGTGATCGACTACAAGATGCCCGACATCAA
TGGAATCGAGCTCGCCAGCCGCCTGCGTAAATCGGACGGCGAAACGCCCGTGATCCTGATCACCGGCTAT
CCGGACGAAAACATCTCGACCCGGGCCGCCGCGGCCGGCGTAAAAGACGTGGTTTTGAAGCCGCTTCTCG
ACGAAAACCTGCTCAAGCGTATCCGCCGCGCCATCCAGGACCGGCCTCGGGCATGACCTACGGGGTTCTA
CGTAAGGCACCCCCCTTAAGATATCGCTCGAAATTTTCGAACCTCCCGATACCGCGTACCAATGCGTCAT
CACAACGGAGATGGCGCAGATGCTGACCCAGACACTCAAGACCCAGGTGATCAACACCCAAATCGGTGGC
AAGATTGCCCCGCCCCATCA

By “FixLJ” is meant a two-component protein system that includes FixL and FixJ polypeptides and has nitrogen fixing activity.

By “fixLJ polynucleotide” is meant a polynucleotide encoding the components of the FixLJ pathway.

By “FixJ polypeptide” is meant a protein having at least about 85% amino acid sequence identity to an amino acid sequence provided at GenBank Accession Nos. WP_085039245.1, WP_228416042.1, WP_226189216.1, WP_226168723.1, WP_217255336.1, WP_217041212.1, WP_212197479.1, WP_212159176.1, WP_212113432.1, WP_006493245.1, QTO47407.1, WP_176091275.1, WP_175858202.1, WP_174990646.1, WP_174391042.1, CAG9265840.1, QVN18508.1, QTO17697.1, CAB3750269.1, QSY15970.1, QSY08188.1, QWJ96640.1, or QRA12269.1 and having transcriptional regulatory function. Exemplary FixJ polypeptide sequences are provided below:

>WP_085039245.1 MULTISPECIES: oxygen response regulator transcription factor
FixJ [Burkholderia cepacia complex]
(SEQ ID NO: 19)
MNSPVTTTQETVFVVDDDEAVRDSLRWLLEANGYRVQCFSSAEQFLDAYQPAQQAGQIACLILDVRMSGM
SGLELQERLIADNAALPIIFVTGHGDVPMAVSTMKKGAMDFIEKPFDEAELRKLVERMLDKARSESKSVQ
EQRAATERLSKLTAREQQVLERIIAGRLNKQIADDLGISIKTVEAHRANIMEKLNVNTVADLLRLALSKK
QA
>WP_228416042.1 oxygen response regulator transcription factor FixJ
[Burkholderia dolosa]
(SEQ ID NO: 20)
MNSPVTTTQETVFVVDDDEAVRDSLRWLQEANGYRVQCFSSAEQFLDAYQPAQQAGQIACLILDVRMSGM
SGLELQERLIADNAALPIIFVTGHGDVPMAVSTMKKGAMDFIEKPFDEAELRKLVERMLDKARSESKSVQ
EQRAASERLSKLTAREQQVLERIIAGRLNKQIADDLGISIKTVEAHRANIMEKLNVNTVADLLRLALSKK
QA
>WP_226189216.1 oxygen response regulator transcription factor FixJ
[Burkholderia vietnamiensis]
(SEQ ID NO: 21)
MNSPVTTTQETVFVVDDDEAVRDSLRWLLEANGYRVQCFSSAEQFLDAYQPAQQAGQIACLILDVRMSGM
SGLELQERLIADNAALPIIFVTGHGDVPMAVSTMKKGAMDFIEKPFDEAELRKLVERMLDKARSESKSVQ
EQRAASERLSKLTAREQRVLERIIAGRLNKQIADDLGISIKTVEAHRANIMEKLNVNTVADLLRLALSKK
QA
>WP_226168723.1 oxygen response regulator transcription factor FixJ
[Burkholderia cepacia]
(SEQ ID NO: 22)
MNSPVTTTQETVFVVDDDEAVRDSLRWLLEANGYRVQCFSSAEQFLDAYQPAQQAGQVACLILDVRMSGM
SGLELQERLIADNAALPIIFVTGHGDVPMAVSTMKKGAMDFIEKPFDEAELRKLVERMLDKARSESKSVQ
EQRAASERLSKLTAREQQVLERIIAGRLNKQIADDIGISIKTVEAHRANIMEKLNVNTVADLLRLALSKK
QA
>WP_217255336.1 oxygen response regulator transcription factor FixJ
[Burkholderia multivorans]
(SEQ ID NO: 23)
MNSPVTTTQETVFVVDDDEAVRDSLRWLLEANGYRVQCFSSAEQFLDAYQPAQQAGQIACLILDVRMSGM
SGLELQERLIADNAALPIIFVTGHGDVPMAVSTMKKGAMDFVEKPFDEAELRKLVERMLDKARSESKSVQ
EQRAASERLSKLTAREQQVLERIIAGRLNKQIADDLGISIKTVEAHRANIMEKLNVNTVADLLRLALSKK
PA
>WP_217041212.1 oxygen response regulator transcription factor FixJ
[Burkholderia multivorans]
(SEQ ID NO: 24)
MNSPVTTTQETVFVVDDDEAVRDSLRWLLEANGYRVQCFSSAEQFLDAYQPAQQAGQIACLILDVRMSGM
SGLELQERLIADNAALPIIFVTGHGDVPMAVSTMKKGAMDFIEKPFDEAELRKLVERMLDKARSESKSVQ
EQRAASERLSKLTAREQQVLERIIAGRLNKQIADDLGISIKTVEAHRANIMEKLNVNTVADLLRLALSKN
PA
>WP_212197479.1 oxygen response regulator transcription factor FixJ
[Burkholderia cenocepacia]
(SEQ ID NO: 25)
MNSPVTTTQETVFVVDDDEAVRDSLRWLLEANGYRVQCFSSAEQFLDAYQPAQQAGQIACLILDVRMSGM
SGLELQERLIADNAALPIIFVTGHGDVPMALSTMKKGAMDFIEKPFDEAELRKLVERMLDKARSESKSVQ
EQRAASERLSKLTAREQQVLERIIAGRLNKQIADDLGISIKTVEAHRANIMEKLNVNTVADLLRLALSKK
QA
>WP_212159176.1 oxygen response regulator transcription factor FixJ
[Burkholderia vietnamiensis]
(SEQ ID NO: 26)
MNSPVTTTQETVFVVDDDEAVRDSLRWLLEANGYRVQCFSSAEQFLDAYQPAQQAGQIACLILDVRMSGM
SGLELQERLIADNAALPIIFVTGHGDVPMAVSTMKKGAMDFIEKPFDEAELRKLVERMLDKARSESKSVQ
EQRAASERVSKLTAREQQVLERIIAGRLNKQIADDLGISIKTVEAHRANIMEKLNVNTVADLLRLALSKK
QA
>WP_212113432.1 oxygen response regulator transcription factor FixJ
[Burkholderia cenocepacia]
(SEQ ID NO: 27)
MNSPVTTTQETVFVVDDDEAVRDSLRWLLEANGYRVQCFSSAEQFLDAYQPAQQAGQIACLILDVRMSGM
SGLELQERLIADNAALPIIFVTGHGDVPMAVSTMKKGAMDFIEKPFDEAELRKLVERMLDKARSESKSVQ
EQRAASERLSKLTAREQQVLERIIAGRLNKQIADVLGISIKTVEAHRANIMEKLNVNTVADLLRLALSKK
QA
>WP_006493245. 1 MULTISPECIES: oxygen response regulator transcription factor
FixJ [Burkholderia]
(SEQ ID NO: 28)
MNSPVTTTQETVFVVDDDEAVRDSLRWLLEANGYRVQCFSSAEQFLDAYQPAQQAGQIACLILDVRMSGM
SGLELQERLIADNAALPIIFVTGHGDVPMAVSTMKKGAMDFIEKPFDEAELRKLVERMLDKARSESKSVQ
EQRAASERLSKLTAREQQVLERIIAGRLNKQIADDLGISIKTVEAHRANIMEKLNVNTVADLLRLALSKK
QA
>QT047407.1 oxygen response regulator transcription factor FixJ [Burkholderia
latens]
(SEQ ID NO: 29)
MNSPVTTTQETVFVVDDDEAVRDSLRWLLEANGYRVQCFSSAEQFLDAYQPAQQAGQIACLILDVRMSGM
SGLELQERLIADNAALPIIFVTGHGDVPMAVSTMKKGAMDFIEKPFDEAELRKLVERMLDKARSESKSVQ
EQRAASERLSKLTAREQQVLERIIAGRLNKQIADDLGISIKTVEAHRANIMEKLNVNTVADLLRLALSKK
QA
>WP_176091275.1 oxygen response regulator transcription factor FixJ
[Burkholderia ambifaria]
(SEQ ID NO: 30)
MNSPVTTTQETVFVDDDDEAVRDSLRWLLEANGYRVQCFSSAEQFLDAYQPAQQAGQIACLILDVRMSGM
SGLELQERLIADNAALPIIFVTGHGDVPMAVSTMKKGAMDFIEKPFDEAELRKLVERMLDKARSESKSVQ
EQRAASERLSKLTAREQQVLERIIAGRLNKQIADDLGISIKTVEAHRANIMEKLNVNTVADLLRLALSKK
QA
>WP_175858202.1 oxygen response regulator transcription factor FixJ
[Burkholderia anthina]
(SEQ ID NO: 31)
MNSPVTTTQETVFVVDDDEAVRDSLRWLLEANGYRVQCFSSAEQFLDAYQPAQQAGQIACLILDVRMSGM
SGLELQERLIADNAALPIIFVTGHGDVPMTVSTMKKGAMDFIEKPFDEAELRKLVERMLDKARSESKSVQ
EQRAASERLSKLTAREQQVLERIIAGRLNKQIADDLGISIKTVEAHRANIMEKLNVNTVADLLRLALSKK
QA
>WP_174990646.1 oxygen response regulator transcription factor FixJ
[Burkholderia contaminans]
(SEQ ID NO: 32)
MNSPVTTTQETVFVVDDDEAVRDSLRWLLEANGYRVQCFSSAEQFLDAYQPAQQAGQIACLILDVRMSGM
SGLELQERLIADNAALPIIFVTGHGDVPMAVSTMKKGAMDFIEKPFDEAELRKLVERMLDKARSESKSVQ
EQRAASERLSKLTAREQQVHERIIAGRLNKQIADDLGISIKTVEAHRANIMEKLNVNTVADLLRLALSKK
QA
>WP_174391042.1 oxygen response regulator transcription factor FixJ
[Burkholderia metallica]
(SEQ ID NO: 33)
MNSPVTTTQETVFVVDDDEAVRDSLRWLLEANGYRVQCFSSAEQFIDAYQPAQQAGQIACLILDVRMSGM
SGLELQERLIADNAALPIIFVTGHGDVPMAVSTMKKGAMDFIEKPFDEAELRKLVERMLDKARSESKSVQ
EQRAASERLSKLTAREQQVLERIIAGRLNKQIADDLGISIKTVEAHRANIMEKLNVNTVADLLRLALSKK
QA
>CAG9265840.1 Transcriptional regulatory protein FixJ [Burkholderia diffusa]
(SEQ ID NO: 34)
MNSPVTTTQETVFVVDDDEAVRDSLRWLLEANGYRVQCFSSAEQFLDAYQPAQQAGQIACLILDVRMSGM
SGLELQERLIADNAALPIIFVTGHGDVPMAVSTMKKGAMDFIEKPFDEAELRKLVERMLDKARSESKSVQ
EQRAASERLSKLTAREQQVLERIIAGRLNKQIADDLGISIKTVEAHRANIMEKLNVNTVADLLRLALSKK
QA
>QVN18508.1 oxygen response regulator transcription factor FixJ [Burkholderia
pyrrocinia]
(SEQ ID NO: 35)
MNSPVTTTQETVFVVDDDEAVRDSLRWLLEANGYRVQCFSSAEQFLDAYQPAQQAGQIACLILDVRMSGM
SGLELQERLIADNAALPIIFVTGHGDVPMAVSTMKKGAMDFIEKPFDEAELRKLVERMLDKARSESKSVQ
EQRAASERLSKLTAREQQVLERIIAGRLNKQIADDLGISIKTVEAHRANIMEKLNVNTVADLLRLALSKK
QA
>QT017697.1 oxygen response regulator transcription factor FixJ [Burkholderia
seminalis]
(SEQ ID NO: 36)
MNSPVTTTQETVFVVDDDEAVRDSLRWLLEANGYRVQCFSSAEQFLDAYQPAQQAGQIACLILDVRMSGM
SGLELQERLIADNAALPIIFVTGHGDVPMAVSTMKKGAMDFIEKPFDEAELRKLVERMLDKARSESKSVQ
EQRAASERLSKLTAREQQVLERIIAGRLNKQIADDIGISIKTVEAHRANIMEKLNVNTVADLLRLALSKK
QA
>CAB3750269.1 Transcriptional regulatory protein FixJ [Burkholderia puraquae]
(SEQ ID NO: 37)
MNEALHSTPDASVVAIVDDDEAVRDGLALLLHSVGLATRCYADAQAFLVDADDAALGCVLLDIRMPGMSG
LDALDELRARRDLPAIVMTGHGNIDACRRAFKRGALDFLRKPVDDDELIDAVQQAIRRHAAQREQGAHGG
ADPARAARIATLSAREREVLDGIVRGWSNKEIARELGLSPRTVETYRANVFDKLQAASLVELVREYAAFA
GAASP
>QSY15970.1 oxygen response regulator transcription factor FixJ [Burkholderia
pseudomallei]
(SEQ ID NO: 38)
MNSPVTTSQETVFVVDDDEAVRDSLRWLLEANGYRVQCFSSAEQFLDAYQPAQQAGQIACLILDVRMSGM
SGLELQERLIAENAALPIIFVTGHGDVPMAVSTMKKGAMDFIEKPFDEAELRKLVERMLEKARNESKSVQ
EQRAASERLSKLTAREQQVLERIIAGRLNKQIADDLGISIKTVEAHRANIMEKLNVNTVADLLRLALSKK
QA
>QSY08188.1 oxygen response regulator transcription factor FixJ [Burkholderia
pseudomallei]
(SEQ ID NO: 38)
MNSPVTTSQETVFVVDDDEAVRDSLRWLLEANGYRVQCFSSAEQFLDAYQPAQQAGQIACLILDVRMSGM
SGLELQERLIAENAALPIIFVTGHGDVPMAVSTMKKGAMDFIEKPFDEAELRKLVERMLEKARNESKSVQ
EQRAASERLSKLTAREQQVLERIIAGRLNKQIADDLGISIKTVEAHRANIMEKLNVNTVADLLRLALSKK
QA
>QWJ96640. 1 oxygen response regulator transcription factor FixJ [Burkholderia
pseudomallei]
(SEQ ID NO: 38)
MNSPVTTSQETVFVVDDDEAVRDSLRWLLEANGYRVQCFSSAEQFLDAYQPAQQAGQIACLILDVRMSGM
SGLELQERLIAENAALPIIFVTGHGDVPMAVSTMKKGAMDFIEKPFDEAELRKLVERMLEKARNESKSVQ
EQRAASERLSKLTAREQQVLERIIAGRLNKQIADDLGISIKTVEAHRANIMEKLNVNTVADLLRLALSKK
QA
>QRA12269.1 oxygen response regulator transcription factor FixJ [Burkholderia
thailandensis]
(SEQ ID NO: 39)
MNSPVTTSQETVFVVDDDEAVRDSLRWLLEANGYRVQCFSSAEQFLEAYQPAQQAGQIACLILDVRMSGM
SGLELQERLIAENAALPIIFVTGHGDVPMAVSTMKKGAMDFIEKPFDEAELRKLVERMLDKARSESKSVQ
EQRAASERLSKLTAREQQVLERIIAGRLNKQIADDLGISIKTVEAHRANIMEKLNVNTVADLLRLALSKK
QA

By “FixJ polynucleotide” is meant a nucleic acid molecule encoding a FixJ polypeptide. In an embodiment, the FixJ polynucleotide is a FixLJ polynucleotide. The sequence of an exemplary FixJ (FixLJ) polynucleotide is provided herein above. See NCBI Accession No. X56808.1.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “decreases” is meant a reduction by at least about 5% relative to a reference level. A decrease may be by 5%, 10%, 15%, 20%, 25% or 50%, or even by as much as 75%, 85%, 95% or more and any intervening percentages.

“Detect” refers to identifying the presence, absence or amount of the analyte to be detected. In some embodiments, the analyte is a polypeptide or polynucleotide associated with the FixLJ pathway.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include bacterial infections associated with a bacteria having a FixLJ pathway or a bacteria comprising a protein having an ATP/ADP-binding pocket of a histidine kinase domain capable of binding a BFA agent described herein (e.g., FIG. 1). In an embodiment, the disease is an infection associated with a member of the Burkholderia, Pseudomonas, or Mycobacterium genus.

By “effective amount” is meant the amount of an agent required to ameliorate the symptoms of a disease relative to an untreated patient. In an embodiment, the agent is a BFA compound described in FIG. 1. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In some embodiments, the effective amount is an amount of an agent effective to inhibit or reduce the virulence of a member of the Burkholderia, Pseudomonas, or Mycobacterium genus. In some embodiments, the effective amount is an amount of an agent effective to ameliorate one or more symptoms of an infection associated with a member of the Burkholderia, Pseudomonas, or Mycobacterium genus.

The invention provides a number of targets that are useful for the development of highly specific drugs to treat or ameliorate a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any analyte having an alteration in expression level or activity that is associated with a disease or disorder.

By “mutation” is meant a change in a polypeptide or polynucleotide sequence relative to a reference sequence. In some embodiments, the reference sequence is a wild-type sequence. Exemplary mutations include point mutations, missense mutations, amino acid substitutions, and frameshift mutations. A “loss-of-function mutation” is a mutation that decreases or abolishes an activity or function of a polypeptide. A “gain-of-function mutation” is a mutation that enhances or increases an activity or function of a polypeptide.

As used herein, “obtaining” as in “obtaining an agent” includes synthesizing, purchasing, or otherwise acquiring the agent.

By “portion” is meant a fragment of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 6, 7, 8, 9, 10, 11, 12, 13, 14, 15, 16, 17, 18, 19, 20, or 21 nucleotides.

By “reduces” is meant a negative alteration of at least 10%, 25%, 50%, 75%, or 100%.

By “reference” is meant a standard or control condition. In some embodiments, a reference subject is a healthy subject (e.g., a subject not having a pathogenic infection). In one embodiment, the reference is the growth or virulence of a bacteria in the absence of a therapeutic agent.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30 °C, more preferably of at least about 37 °C, and most preferably of at least about 42 °C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30 °C in 750 mM NaCl, 75 mM trisodium citrate, and 1% SDS. In another embodiment, hybridization will occur at 37 °C in 500 mM NaCl, 50 mM trisodium citrate, 1% SDS, 35% formamide, and 100 μg/ml denatured salmon sperm DNA (ssDNA). In yet another embodiment, hybridization will occur at 42 °C in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25 °C, more preferably of at least about 42 °C, and even more preferably of at least about 68 °C. In another embodiment, wash steps will occur at 25 °C in 30 mM NaCl, 3 mM trisodium citrate, and 0.1% SDS. In yet another embodiment, wash steps will occur at 42 C in 15 mM NaCl, 1.5 mM trisodium citrate, and 0.1% SDS. In another embodiment, wash steps will occur at 68 °C in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison. In embodiments, the reference sequence is a FixL polypeptide, FixJ polypeptide, or FixLJ polynucleotide.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “two-component system” is meant a two protein system used by bacteria to adapt to changes in their environment. Two component systems typically include a pair of at least two proteins. A first protein in the two-component system may be a sensor kinase, which senses external stimuli. A second protein in the two-component system may be a response regulation protein, which alters the expression profile of bacterial genes involved in survival and adaptation. Two-component systems may also be known as “two-component signal transduction systems” or “two-component regulatory systems.” In some embodiments, a two-component system of the present disclosure is a two-component system involved in survival or adaptation of a bacterial cell to a niche found outside of a subject. In some embodiments, a two-component system of the present disclosure is a two-component system involved in microaerobiosis, such as respiratory or nitrogen fixation pathways. In some embodiments, a two-component system of the present disclosure is FixLJ.

By “virulence” is meant the ability of a pathogen to cause harm or damage to a host. In some embodiments, virulence refers to the ability of a member of the Burkholderia, Pseudomonas, or Mycobacterium genus to cause harm or damage to a subject having an infection associated with the member of the Burkholderia, Pseudomonas, or Mycobacterium genus. In an embodiment, virulence is characterized by the ability of a bacterial cell to invade and/or survive within an immune cell, such as a macrophage.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-1D. A high-throughput screen identifies 84 small molecules that activate the Burkholderia FixLJ pathway, 11 of which are fixLJ-specific. FIG. 1A provides a schematic of the high-throughput screen used to identify small molecule activators of the FixLJ pathway. FIG. 1B provides a scatter plot from a high-throughput screen of 28,100 compounds for activators of GFP activity in B. multivorans strain VC7102 carrying a GFP reporter for FixLJ pathway activity. Dots are averages of two replicate wells treated with same compound. Hits (gray dots) have that GFP activity more than 3 standard deviations above the plate-specific mean negative control wells (DMSO, black dots). FIG. 1C provides a scatter plot of 84 hits from the primary screen chosen for follow-up assays measuring the GFP signal in B. dolosa and its fixLJ deletion mutant to assess dependence on FixLJ pathway. The GFP as percent of negative control (DMSO-treated) for each compound is plotted as GFP seen the fixLJ deletion mutant vs. parental strain. Type 1 hits (gray squares) have that GFP activity more than 3 standard deviations above the plate-specific mean negative control wells in the parental strain, but not in the fixLJ deletion mutant. Type 2 hits (gray circles) are categorically greater hits in strength in the parental strain compared to the hit strength in the fixLJ deletion mutant. FIG. 1D provides structures of the 10 of the 11 fixLJ-dependent hits. These ten compounds were available from ChemDiv.

FIGS. 2A-2K. BFA (Burkholderia Fix Activator) compounds inhibit B. dolosa virulence in THP-1-derived macrophages in a fixLJ-specific manner. FIGS. 2A-2K provide illustrations and graphs showing that the intracellular survival (and/or uptake) of B. dolosa strain AU0158 in THP-1-derived human macrophages was measured using an antibiotic exclusion assay in the presence of varying concentrations BFA compounds. FIG. 2A provides an illustration showing the experimental procedure. FIGS. 2B-2K provide graphs showing the results after the number of intracellular bacteria was determined by lysing the macrophages and enumerating CFU after incubation for 2 hours (FIGS. 2B and 2D) or 4 hours (FIGS. 2C, 2E, 2F, 2G, 2H, 2I, 2J, and 2K). *, **, and *** denote p<0.05, 0.01, and 0.001, respectively, by two-way ANOVA with Dunnett's multiple comparisons test using 0 μM (DMSO vehicle) as control. Each of FIGS. 2B-2K depict graphs having pairs of bars at each measurement point, where each bar in the pairs of bars corresponds to AU-0158 (left) and AU-0158 AfixLJ (right).

FIGS. 3A-3F. BFA compounds inhibit the virulence of multiple pathogenic Burkholderia species. FIGS. 3A-3F provide graphs showing the results after THP-1-derived macrophages were infected with B. multivorans strain VC7102 (FIGS. 3A and 3D), B. cenocepacia strain K56-2 (FIGS. 3B and 3E), or B. thailandensis strain e264 (FIGS. 3C and 3F) in the presence of 25 μM of BFA compounds (FIGS. 3A-3C) or a dose range of BFA1 (FIGS. 3D-3F). Intracellular bacteria were determined using antibiotic exclusion after 2 (FIG. 3F) or 4 (FIGS. 3A-3E) hour exposure to antibiotic. P value determined by ANOVA with Dunnett's multiple comparisons test, *, **, ***, **** denotes p value <0.05, 0.01, 0.001, 0.0001, respectively.

FIGS. 4A-4B. BFA1 is predicted to bind to FixL at the ATP/ADP-binding pocket of the histidine kinase domain. FIG. 4A provides an illustration showing the predicted binding of BFA1 to first binding pocket of B. dolosa strain AU0158 FixL using AutoDockFR. FIG. 4B provides an illustration showing the proposed mechanism of action for BFA1 activating Burkholderia FixLJ pathway.

FIGS. 5A-5D. Identification of benserazide as fixLJ-independent activator of the FixLJ pathway for use as positive fluorescence control. FIG. 5A provides a scatter plot of the impact of 640 FDA-approved compounds on GFP activity in B. multivorans strain VC7102 carrying GFP reporter for FixLJ pathway activity. Each blue dot depicts the mean of replicate plates containing the same compound. The GFP value was the AGFP value as described in Methods. Both GFP and OD600 are plotted as a % of plate specific-DMSO vehicle (negative) controls depicted in black dots. Benserazide was the only compound for which GFP activity was more than 3 standard deviations above the mean of the negative control (DMSO). FIGS. 5B-5D provide graphs showing dose-dependent benserazide activation of the Fix-GFP reporter plasmid as measured in B. multivorans strain VC7102 (FIG. 5B), B. dolosa strain AU0158 (FIG. 5C), or B. dolosa strain AU0158 AfixLJ (FIG. 5D). Data from each of these strains was measured and plotted as a percent of 0 μM (DMSO only). **** denotes p<0.0001 by ANOVA with Dunnett's Multiple comparison test using 0 μM as control.

FIGS. 6A-6B. BFA compounds are not cytotoxic to human cell lines. FIGS. 6A-6B provide graphs showing the results after varying concentrations of compounds were incubated with A549 epithelial cells (FIG. 6A) or THP-1-derived macrophages (FIG. 6B) for 16 hours. LDH release was measured and plotted as maximum LDH release when cells were treated with detergent to completely lyse cells. Dotted lines denote untreated cells. In FIG. 6A, bars are shown in groups of 4 for each BFA, from left to right, as follows: 0 μM, 6.25 μM, 12.5 μM, and 25 μM. In FIG. 6B, bars are shown in groups of 5 for each BFA, from left to right, as follows: 0 μM, 3.125 μM, 6.25 μM, 12.5 μM, and 25 μM.

FIGS. 7A-7D. In silica binding of BFA1 and ADP to B. dolosa FixL. FIG. 7A provides an illustration showing predicted FixL ligand binding pockets with highest scores. FIG. 7B provides an illustration showing the ADP/FixL complex at ATP/ADP binding site predicted by ADFR (left) and interaction diagram (right). FIG. 7C provides an illustration showing the BFA1/FixL complex at ATP/ADP binding site predicted by ADFR (left) and interaction diagram (right). FIG. 7D provides an illustration showing the BFA1/FixL complex at pocket 2 predicted by ADFR (left) and interaction diagram (right).

FIGS. 8A-8C. BFA compounds reduce P. aeruginosa-induced cytotoxicity. FIGS. 8A-8C provide graphs showing the results after confluent human lung epithelial cells (A549) in a 96 well plate were infected with ~1.5×10³ CFU/well of P. aeruginosa strain PA14 (FIGS. 8A and 8C) or P. aeruginosa strain PA01 and treated with the indicated amount of BFA1 (FIGS. 8A and 8B) or 25 μM of the indicated BFA (FIG. 8C) or DMSO vehicle control for 5.5 hours, when lactate dehydrogenase (LDH) release was measured and percent cytotoxicity was calculated from untreated wells treated with lysis buffer for maximum LDH release. *, **, ***, **** denote P value <0.05, 0.01, 0.001, 0.0001, respectively, by ANOVA with Dunnett's multiple comparison test.

FIG. 9. BFA compounds reduce the virulence of M. abscessus. FIG. 9 provides a graph showing the results after THP-1-derived macrophages were infected with ~5×10⁵ CFU/M. abscessus strain ATCC19977 and 25 μM of the indicated BFA or DMSO vehicle control for 2 hours. Cells were washed and treated with the same BFA and 500 μg/mL amikacin (to kill extracellular bacteria) for 2 hours. Cells were washed, lysed, and intracellular bacteria were enumerated by serial plating. *, **, ***, **** denote P value <0.05, 0.01, 0.001, 0.0001, respectively, by ANOVA with Dunnett's multiple comparison test.

### DETAILED DESCRIPTION OF THE INVENTION

The disclosure features compositions and methods that are useful for inhibiting or reducing the virulence of a bacterial cell and/or treating infections associated with a bacterial cell, such as members of the Burkholderia, Pseudomonas, or Mycobacterium genus.

The invention is based, at least in part, on the discovery that compounds disclosed herein, denoted Burkholderia Fix Activators (BFAs), inhibited the intracellular survival of members of the Burkholderia genus (e.g., in macrophages) without significant toxicity. Without intending to be bound by theory, this inhibition of intracellular survival of members of the Burkholderia genus is likely to be in a fixLJ-dependent manner. One of the compounds, BFA1, inhibited the intracellular survival in macrophages of multiple Burkholderia species. Without intending to be bound by theory, predictive modeling of the interaction of BFA1 with Burkholderia FixL indicates that BFA1 binds to the putative ATP/ADP binding pocket in the kinase domain, indicating a potential mechanism for pathway activation. These results indicate that the small-molecules provided herein (e.g., FixLJ pathway activators) are useful as anti-virulence agents for the treatment of Burkholderia, as well as other bacteria (e.g., Pseudomonas, Mycobacterium) containing a kinase having an ATP/ADP binding pocket that binds BFA compounds.

Burkholderia are intrinsically resistant to multiple antibiotic classes (Rhodes, K. A. & Schweizer, H. P. Drug Resist Updat 28, 82-90 (2016)). This resistance is mediated through alteration of antibiotic targets, decreased outer membrane permeability by modifications of LPS, decreased expression of porins, increased expression of antibiotic-inactivating enzymes, or increased production of efflux pumps (Rhodes, K. A. & Schweizer, H. P. Drug Resist Updat 28, 82-90 (2016); Podnecky, N., Rhodes, K. & Schweizer, H. Frontiers in Microbiology 6, (2015)) In a study of over 2,000 BCC isolates, more than 50% of the isolates were resistant to chloramphenicol, co-trimoxazole, ciprofloxacin, tetracycline, rifampin, and amoxicillin-clavulanate (Zhou, J. et al. Antimicrob Agents Chemother 51, 1085 (2007)). One study of 56 CF isolates of B. dolosa (a BCC member) found them to be nearly pan-resistant, with minocycline being the only active antibiotic (in just 29% of isolates) (Skurnik, D. et al. J Infect Dis 205, 1709-1718 (2012)). Pan-resistance in outbreak strains of B. cenocepacia has also been reported (Sass, A. et al., BMC Genomics 12, 373). While B. pseudomallei isolates are typically susceptible to β-lactam antibiotics (such as ceftazidime, meropenem, imipenem and amoxicillin-clavulanate), these antibiotics are often not effective at clearing the infection, and treatment can last for months. Thus, novel compounds are needed for Burkholderia infections since the number of new prospects from traditional drug development is limited.

The examples of the present disclosure provide a high-throughput screen that identified 11 novel activators of the Burkholderia FixLJ pathway. Eight of these compounds were able to inhibit the virulence of Burkholderia in a fixLJ-dependent manner in vitro using macrophage and epithelial intracellular survival assay. The most active compound inhibited multiple Burkholderia species, including B. thailandensis, a model organism for B. pseudomallei.

This invention is also based, at least in part, on the discovery that the BFAs disclosed herein also have anti-virulence effects when other bacterial species, such as members of the Pseudomonas or Mycobacterium genus were contacted with the BFAs. Without intending to be bound by theory, since members of the Pseudomonas or Mycobacterium genus lack the FixLJ pathway, this general anti-virulence effect is hypothesized to be due to the effect of BFAs in generally activating two-component systems in bacterial cells. Since two-component systems are widely found in bacteria, these results indicate that the small-molecules provided herein (e.g., the BFAs) are promising generally applicable anti-virulence agents for bacterial cells (e.g., Burkholderia, Pseudomonas, Mycobacterium).

### Two-Component System

Two-component systems are protein systems used by bacteria to adapt to changes in their environment. Two component systems typically include a pair of at least two proteins. A first protein in the two-component system may be a sensor kinase, which senses external stimuli. A second protein in the two-component system may be a response regulation protein, which alters the expression profile of bacterial genes involved in survival and adaptation. Two-component systems may also be known as “two-component signal transduction systems” or “two-component regulatory systems.”

In some embodiments, a two-component system of the present disclosure is a two-component system involved in survival or adaptation of a bacterial cell to a niche found outside of a subject. In some embodiments, a two-component system of the present disclosure is a two-component system involved in microaerobiosis, such as respiratory or nitrogen fixation pathways. In some embodiments, a two-component system of the present disclosure is FixLJ.

### FixLJ

FixLJ is a two-component system (TCS) comprising FixL and FixJ, and is encoded by fixLJ. Two-component systems are one mechanism that bacteria use to sense and respond to their environment by modulating gene expression. Burkholderia fixLJ was previously identified as a two-component system in bacterial whole-genome sequencing studies that is under strong positive selection during chronic B. dolosa or B. multivorans infection in people with CF (Lieberman, T. D. et al. Nature genetics 46, 82-87 (2014); Lieberman, T. D. et al. Nature genetics 43, 1275-1280 (2011); Silva, I. N. et al. mSystems 1, e00029-00016 (2016)). The fixLJ system regulates ~11% of the genome of B. dolosa (Schaefers, M. M. et al. PLOS Pathog 13, e1006116 (2017)) and is required for virulence.

FixL is a dimeric heme protein kinase that senses the oxygen level in plant root nodules to regulate the transcription of nitrogen fixation genes via the phosphorylation of its cognate transcriptional activator. Dissociation of oxygen from the heme induces conformational changes in the protein, converting it from the inactive form for phosphorylation to the active form. Under low oxygen conditions, FixL autophosphorylates and transmits phosphate to the FixJ response regulator.

FixJ is a transcriptional regulator that has been shown to control 74% of the genes induced in microaerobiosis (2% oxygen) and the majority of genes expressed in mature bacteroids, in Sinorhizobium meliloti. In Sinorhizobium meliloti, once FixJ is phosphorylated by FixL, FixJ activates transcription of the nifA and fixK genes, encoding two intermediate regulators which induce expression of nif and fix genes involved in respiration and nitrogen fixation.

Otherwise isogenic BCC constructs carrying evolved (late) fixL sequence variants have also been shown to be more virulent than constructs carrying ancestral (early) fixL sequence variants (Schaefers, M. M. et al., PLOS Pathog 13, e1006116 (2017)). Interestingly, bacteria carrying these evolved fixL sequence variants have lower levels of FixLJ pathway activity, demonstrating that high levels of FixLJ pathway activity are detrimental to virulence.

### Methods of Inhibition or Reduction of Virulence

The present disclosure provides methods of inhibiting or reducing the virulence of a bacterial cell. In some embodiments, the bacterial cell is a member of the Burkholderia, Pseudomonas, or Mycobacterium genus, such as a bacterium disclosed in Table 3 of the present disclosure. These methods include contacting a bacterial cell with an amount of an agent of the present disclosure, or a pharmaceutical composition comprising that agent, sufficient to reduce or inhibit the virulence of the bacterial cell. These methods also include administering to a subject an amount of an agent of the present disclosure, or a pharmaceutical composition thereof, sufficient to reduce or inhibit the virulence of the bacterial cell. In some embodiments, the agent is an activator of a two-component system. In some embodiments, the agent is a FixLJ pathway activator. In some embodiments, the agent is an agent disclosed in Table 5 of the present disclosure.

Virulence, as used herein, is the ability of a pathogen or bacterial cell to damage or harm a subject. The ability of a pathogen or bacterial cell to cause disease is thus linked to its pathogenicity. As applied to bacterial cells, virulence can be characterized in terms of virulence factors, including, but not limited to, ability to infect a subject, immunoevasive ability, and immunosuppressive ability. Once the subject has been infected, virulence can also be characterized in the ability of a bacterial cell to successfully colonize and survive in a niche within the subject. Accordingly, inhibition or reduction of virulence of a bacterial cell may be characterized through reduction of any of the virulence factors. For example, a reduction in virulence of a bacterial cell may result in a reduction of the ability of the bacterial cell to infect a subject, evade the subject's immune system, suppress the subject's immune system, or otherwise survive within the subject. In some embodiments, virulence factors include factors associated with bacterial pathogenesis or toxicity, whether associated with bacterial toxins or host-mediated responses to bacterial cells. An exemplary virulence factor associated with bacterial pathogenesis or toxicity is, for example, tissue damage (i.e., cytotoxicity) in a host in response to bacterial infection.

### Methods of Treatment, Delivery, and Administration

The present disclosure provides methods of treating disease and/or disorders, such as bacterial infections, or symptoms thereof which comprise administering a therapeutically effective amount of a pharmaceutical composition comprising an agent which inhibits or reduces the virulence of a bacterial cell (e.g., member of the Burkholderia, Pseudomonas, or Mycobacterium genus). Thus, one embodiment is a method of treating a subject suffering from an infection associated with a bacterial cell (e.g., member of the Burkholderia, Pseudomonas, or Mycobacterium genus), or symptom thereof. The method includes the step of administering to the mammal a therapeutic amount of an amount of an agent which inhibits or reduces the virulence of a bacterial cell (e.g., member of the Burkholderia, Pseudomonas, or Mycobacterium genus), or a pharmaceutical composition comprising an agent which inhibits or reduces the virulence of a bacterial cell (e.g., member of the Burkholderia, Pseudomonas, or Mycobacterium genus), as disclosed herein, sufficient to treat the disease or disorder or symptom thereof, under conditions such that the disease or disorder is treated.

The methods herein include administering to the subject (including a subject identified as in need of such treatment) an effective amount of an agent which inhibits or reduces the virulence of a bacterial cell (e.g., member of the Burkholderia, Pseudomonas, or Mycobacterium genus), or a pharmaceutical composition comprising an agent which inhibits or reduces the virulence of a bacterial cell (e.g., member of the Burkholderia, Pseudomonas, or Mycobacterium genus), described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of an agent which inhibits or reduces the virulence of a bacterial cell (e.g., member of the Burkholderia, Pseudomonas, or Mycobacterium genus), or a pharmaceutical composition comprising an agent which inhibits or reduces the virulence of a bacterial cell (e.g., member of the Burkholderia, Pseudomonas, or Mycobacterium genus), as disclosed herein, to a subject (e.g., animal, human) in need thereof, including a mammal, particularly a human. Such treatment will be suitably administered to subjects, particularly humans, suffering from, having, susceptible to, or at risk for infection associated with a bacterial cell (e.g., a member of the Burkholderia, Pseudomonas, or Mycobacterium genus), or symptom thereof. Determination of those subjects “at risk” can be made by any objective or subjective determination by a diagnostic test or opinion of a subject or health care provider (e.g., genetic test, enzyme or protein marker, marker, family history, and the like). The agents and compositions disclosed herein may be also used in the treatment of any other disorders in which infection associated with a bacterial cell (e.g., a member of the Burkholderia, Pseudomonas, or Mycobacterium genus), may be implicated.

In another embodiment, a method of monitoring the progress of an infection associated with a bacterial cell (e.g., a member of the Burkholderia, Pseudomonas, or Mycobacterium genus), or symptom thereof, or monitoring treatment of the disease is provided. The method includes a diagnostic measurement (e.g., bacterial culture, activation or expression assay for a two component system, such as the FixLJ pathway) in a subject suffering from the disease or symptoms thereof, in which the subject has been administered an amount (e.g., a therapeutic amount) of an agent which inhibits or reduces the virulence of a bacterial cell, such as a bacterial cell (e.g., a member of the Burkholderia, Pseudomonas, or Mycobacterium genus), or a pharmaceutical composition thereof, as described herein, sufficient to treat the disease or symptoms thereof. The diagnostic measurement in the method can be compared to samples from healthy, normal controls; in a pre-disease sample of the subject; or in other afflicted/diseased patients to establish the treated subject's disease status. For monitoring, a second diagnostic measurement may be obtained from the subject at a time point later than the determination of the first diagnostic measurement, and the two measurements can be compared to monitor the course of disease or the efficacy of the therapy/treatment. In certain embodiments, a pre-treatment measurement in the subject (e.g., in a sample or biopsy obtained from the subject) is determined prior to beginning treatment as described; this measurement can then be compared to a measurement in the subject after the treatment commences and/or during the course of treatment to determine the efficacy of (monitor the efficacy of) the disease treatment.

The agent which inhibits or reduces the virulence of bacterial cell (e.g., a member of the Burkholderia, Pseudomonas, or Mycobacterium genus), or pharmaceutical compositions thereof, can be administered to a subject by any of the routes normally used for introducing a small molecule, such as an antimicrobial, or composition containing the small molecule into a subject. Routes and methods of administration include, without limitation, intradermal, intramuscular, intraperitoneal, intrathecal, parenteral, such as intravenous (IV) or subcutaneous (SC), vaginal, rectal, intranasal, inhalation, intraocular, intracranial, or oral. Parenteral administration, such as subcutaneous, intravenous or intramuscular administration, is generally achieved by injection (immunization). Injectables can be prepared in conventional forms and formulations, either as liquid solutions or suspensions, solid forms (e.g., lyophilized forms) suitable for solution or suspension in liquid prior to injection, or as emulsions. Injection solutions and suspensions can be prepared from sterile powders, granules, and tablets. Administration can be systemic or local.

The agent which inhibits or reduces the virulence of a bacterial cell (e.g., a member of the Burkholderia, Pseudomonas, or Mycobacterium genus), or pharmaceutical compositions thereof, can be administered in any suitable manner, such as with pharmaceutically acceptable carriers, diluents, or excipients as described supra. Pharmaceutically acceptable carriers are determined in part by the particular composition being administered, as well as by the particular method used to administer the composition. Accordingly, a pharmaceutical composition comprising the agent which inhibits or reduces the virulence of a bacterial cell (e.g., a member of the Burkholderia, Pseudomonas, or Mycobacterium genus), can be prepared using a wide variety of suitable and physiologically and pharmaceutically acceptable formulations.

Administration of the agent which inhibits or reduces the virulence of a bacterial cell (e.g., a member of the Burkholderia, Pseudomonas, or Mycobacterium genus), or pharmaceutical compositions thereof, can be accomplished by single or multiple doses. The dose administered to a subject should be sufficient to induce a beneficial therapeutic response in a subject over time, such as to inhibit, block, reduce, ameliorate, protect against, or prevent a cardiac disease or disorder (e.g., a cardiac disorder involving abnormal force production in cardiac muscle, cardiac hypertrophy, or hypertrophic cardiomyopathy), or symptom thereof. The dose required will vary from subject to subject depending on the species, age, weight and general condition of the subject, by the severity of the cancer being treated, by the particular composition being used and by the mode of administration. An appropriate dose can be determined by a person skilled in the art, such as a clinician or medical practitioner, using only routine experimentation. One of skill in the art is capable of determining therapeutically effective amounts of the agent which inhibits or reduces the virulence of a bacterial cell (e.g., a member of the Burkholderia, Pseudomonas, or Mycobacterium genus), or pharmaceutical compositions thereof, that provide a therapeutic effect or protection against an infection associated with a bacterial cell (e.g., a member of the Burkholderia, Pseudomonas, or Mycobacterium genus), or symptom thereof, suitable for administering to a subject in need of treatment or protection.

In some embodiments, an agent which inhibits or reduces the virulence of a bacterial cell (e.g., a member of the Burkholderia, Pseudomonas, or Mycobacterium genus), or a pharmaceutical composition thereof, is administered as a maximum-tolerated dose (MTD). In some embodiments, MTD is the dose with estimated probability of dose limiting toxicity (DLT) closest to the target toxicity rate of 20%. In some embodiments, an agent which inhibits or reduces the virulence of a bacterial cell (e.g., a member of the Burkholderia, Pseudomonas, or Mycobacterium genus), or a pharmaceutical composition thereof, is administered in a therapeutically effective dose for a mammal. In some embodiments, the mammal is a mouse. In some embodiments, the mammal is a human.

In some embodiments, the bacterial cell is a member of the Burkholderia, Pseudomonas, or Mycobacterium genus, such as a bacterium disclosed in Table 3 of the present disclosure.

In some embodiments, the agent is an activator of a two-component system. In some embodiments, the agent is a FixLJ pathway activator. In some embodiments, the agent is an agent disclosed in Table 5 of the present disclosure.

### Pharmaceutical Compositions

Compositions comprising an agent which inhibits or reduces the virulence of a bacterial cell (e.g., a member of the Burkholderia, Pseudomonas, or Mycobacterium genus), as described herein are provided. In some embodiments, the compositions further comprise a pharmaceutically acceptable carrier, diluent, excipient, or vehicle. In some embodiments, an adjuvant (a pharmacological or immunological agent that modifies or boosts an immune response, e.g., to produce more antibodies that are longer-lasting) is also employed. For example, without limitation, the adjuvant can be an inorganic compound, such as alum, aluminum hydroxide, or aluminum phosphate; mineral or paraffin oil; squalene; detergents such as Quil A; plant saponins; Freund's complete or incomplete adjuvant, a biological adjuvant (e.g., cytokines such as IL-1, IL-2, or IL-12); bacterial products such as killed Bordetella pertussis, or toxoids; or immunostimulatory oligonucleotides (such as CpG oligonucleotides). In some embodiments, the adjuvant is conjugated to an amphiphile as previously described (H. Liu et al., Structure-based programming of lymph-node targeting in molecular vaccines. Nature 507, 5199522 (2014)). In some embodiments, the amphiphile is N-hydroxy succinimidyl ester-end-functionalized poly(ethylene glycol)-lipid (NHS-PEG2KDa-DSPE).

Compositions and preparations (e.g., physiologically or pharmaceutically acceptable compositions) containing an agent which inhibits or reduces the virulence of a bacterial cell (e.g., a member of the Burkholderia, Pseudomonas, or Mycobacterium genus) for parenteral administration include, without limitation, sterile aqueous or non-aqueous solutions, suspensions, and emulsions. Nonlimiting examples of non-aqueous solvents include propylene glycol, polyethylene glycol, vegetable oils, such as olive oil and canola oil, and injectable organic esters, such as ethyl oleate. Aqueous carriers include water, alcoholic/aqueous solutions, emulsions, or suspensions, including saline and buffered media. Parenteral vehicles include, for example, sodium chloride solution, Ringer's dextrose, dextrose and sodium chloride, lactated Ringer's, or fixed oils. Intravenous vehicles include, for example, fluid and nutrient replenishers, electrolyte replenishers (such as those based on Ringer's dextrose), and the like. Preservatives and other additives may also be present in such compositions and preparations, such as, for example, antimicrobials, antioxidants, chelating agents, colorants, stabilizers, inert gases, and the like.

Some of the compositions may potentially be administered as a pharmaceutically acceptable acid- or base-addition salt, formed by reaction with inorganic acids, such as hydrochloric acid, hydrobromic acid, perchloric acid, nitric acid, thiocyanic acid, sulfuric acid, and phosphoric acid, and organic acids, such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, tri-alkyl and aryl amines and substituted ethanolamines.

Provided herein are pharmaceutical compositions which include a therapeutically effective amount of an agent which inhibits or reduces the virulence of a bacterial cell (e.g., a member of the Burkholderia, Pseudomonas, or Mycobacterium genus), as described herein, alone, or in combination with a pharmaceutically acceptable carrier. Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid or aqueous solution, suspension, emulsion, dispersion, tablet, pill, capsule, powder, or sustained release formulation. A liquid or aqueous composition can be lyophilized and reconstituted with a solution or buffer prior to use. The composition can be formulated as a suppository, with traditional binders and carriers such as triglycerides. Oral formulations can include standard carriers, such as pharmaceutical grades of mannitol, lactose, starch, magnesium stearate, sodium saccharine, cellulose, and magnesium carbonate. Any of the commonly known pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives, and the like. Other media that can be used in the compositions and administration methods as described are normal saline and sesame oil.

### Kits

The disclosure also provides kits for use in inhibiting or reducing the virulence of a bacteria (e.g., a member of the Burkholderia, Pseudomonas, or Mycobacterium genus). In some embodiments, the kits provided are for use in treating a pathogenic infection, such as an infection associated with a bacteria (e.g., a member of the Burkholderia, Pseudomonas, or Mycobacterium genus) in a subject. Kits of the instant disclosure may include one or more containers comprising an agent for inhibiting or reducing the virulence of a member of the Burkholderia, Pseudomonas, or Mycobacterium genus. Kits of the instant disclosure may include one or more containers comprising an agent which activates a two-component system, such as an activator of the FixLJ pathway. In some embodiments, the kits further include instructions for use in accordance with the methods of this disclosure. In some embodiments, these instructions comprise a description of use of the agent to inhibit or reduce the virulence of a member of the Burkholderia, Pseudomonas, or Mycobacterium genus, or a description of use of the agent to treat a subject having a pathogenic infection, such as an infection associated with a member of the Burkholderia, Pseudomonas, or Mycobacterium genus.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. Normally, the kit comprises a container and a label or package insert(s) on or associated with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: High-Throughput Screen Identifies 84 Compounds that Activate FixLJ Pathway

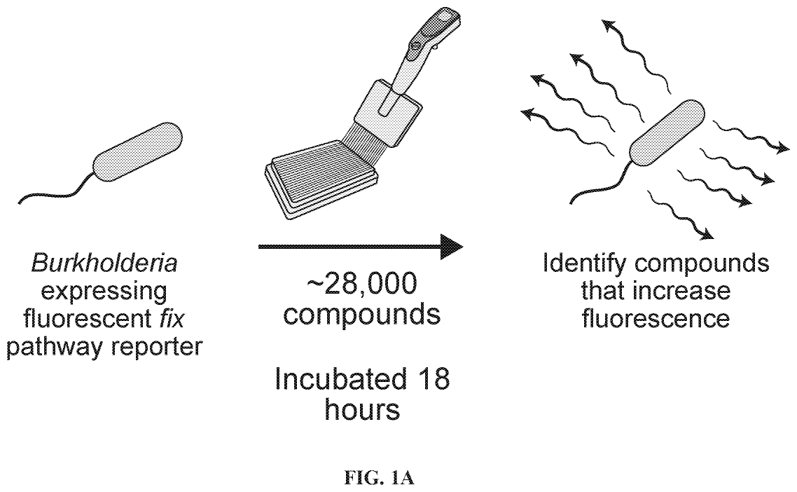

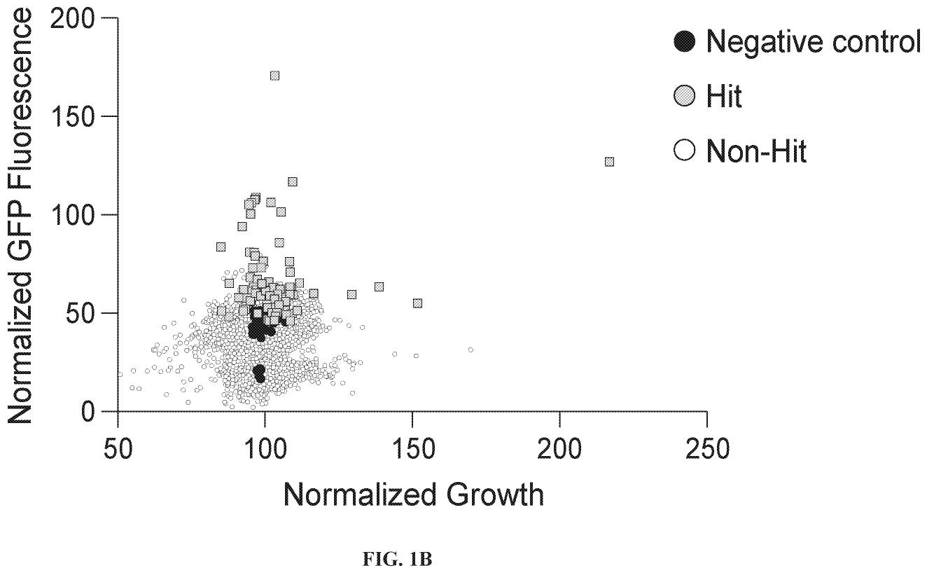

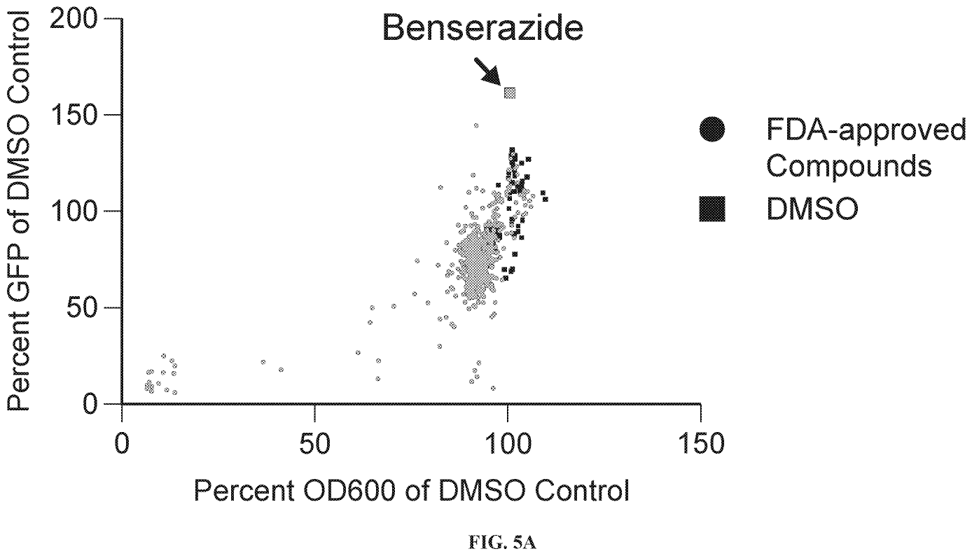

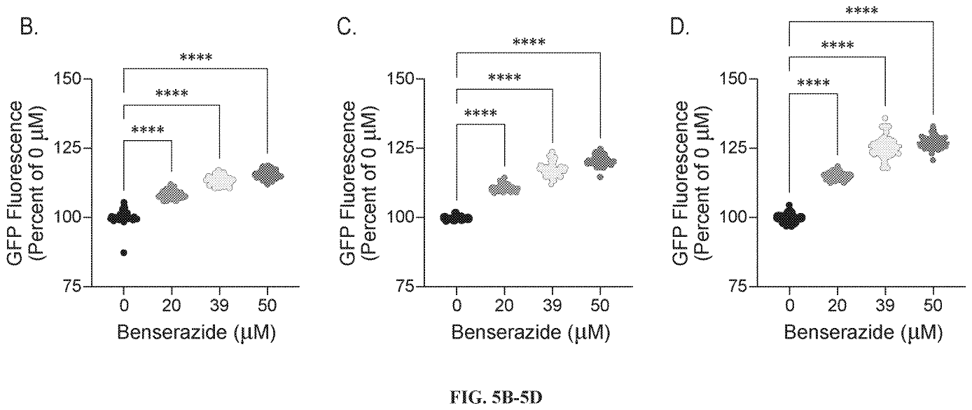

A high-throughput screen was conducted to look for activators of the Burkholderia FixLJ pathway with the goal of identifying novel anti-virulence compounds. An existing fix pathway reporter (Schaefers, M. M. et al. PLOS Pathog 13, e1006116, (2017); Schaefers, M. M. et al. mBio 12, e01823-01821 (2021)) was modified to express green fluorescent protein (GFP) instead of LacZ when the fixK promoter is activated by FixLJ. This construct was cloned into a mini-Tn7 based vector, allowing for stable integration of the reporter into the Burkholderia chromosome without antibiotic selection. This reporter was then conjugated into the clinical cystic fibrosis (CF) isolate B. multivorans strain VC7102. For the screen, this reporter strain was incubated in 384-well plates in the presence of compounds or DMSO vehicle control, and both OD600 and GFP fluorescence were measured after overnight growth at 37 °C (FIG. 1A). In initial screens a library of 640 FDA-approved compounds was tested for their ability to induce the FixLJ pathway. Among this library were several antibiotics and other compounds that inhibited bacterial growth and GFP activity (lower left region of FIG. 5A). One compound, an anti-Parkinson's disease drug called benserazide, was identified that was able to induce GFP levels above vehicle (DMSO) treated wells (FIG. 5A). To confirm whether benserazide was a hit across two BCC species, and determine if it was fixLJ-specific, the same GFP reporter conjugated in B. dolosa strain AU0158 and its fixLJ deletion mutant was used. If benserazide specifically targeted FixLJ, there would not be an increase in fluorescence seen in the fixLJ deletion mutant when treated with benserazide. Benserazide was able to induce GFP response in a dose-dependent manner in both B. multivorans and B. dolosa (FIG. 5B), but was it also induced a GFP response in the fixLJ deletion mutant, demonstrating that benserazide activates the GFP reporter in a fixLJ independent mechanism. Benserazide was subsequently used a positive fluorescence control in screening assays.

Next, 28,100 compounds were screened, with each assay plate including at least 1 column (16 wells) that contained DMSO (vehicle) alone as a negative control and at least 1 column (16 wells) that contained benserazide which served as a positive fluorescence control (FIG. 1). Using benserazide as a positive fluorescence control and DMSO as a negative control, Z′ factors ~0.5 were achieved, indicating the screening assay was technically robust. An additional 83 hits having mean fluorescence of the 2 replicate plates at least 3 standard deviations above the plate-specific negative control were identified. Compounds were identified as weak hits if their increase in fluorescence was between 3-6 standard deviations above the negative control mean. Moderate hits had an increase in fluorescence between 6-9 standard deviations, and strong hits were at least 9 standard deviations above the mean negative control value (Table 1). These 83 compounds were “cherry-picked” from compound library plates, and their ability to induce GFP activity in B. dolosa strain AU0158 and its fixLJ deletion mutant was assessed to confirm FixLJ-specific activation of the GFP reporter. It was found that most compounds activated the GFP reporter to similar levels in the parental B. dolosa strain as in the fixLJ deletion mutant, indicating that these compounds were activating the reporter in a FixLJ-independent manner. 7 compounds that activated the GFP reporter only in the parental B. dolosa strain were found, and therefore these compounds were classified as type 1 hits (Gray boxes, FIG. 1C). 4 compounds were also identified that were classified as hits in both the parental B. dolosa strain and the fixLJ deletion mutant, but were a lesser strength hit in the fixLJ deletion mutant compared the parental B. dolosa, so these were classified as type 2 hits (Black dots, FIG. 1C). It is important to note that these hits did not inhibit bacterial growth in the screen. In total 11 compounds were identified that were able to activate the FixLJ pathway (either type 1 or 2 hits), 10 of which were available commercially. The structures of these 10 compounds are depicted in FIG. 1, and full chemical names are listed in Table 5.

### Example 2: Eight of the Small-Molecule FixLJ Activators Inhibit Burkholderia Virulence In Vitro

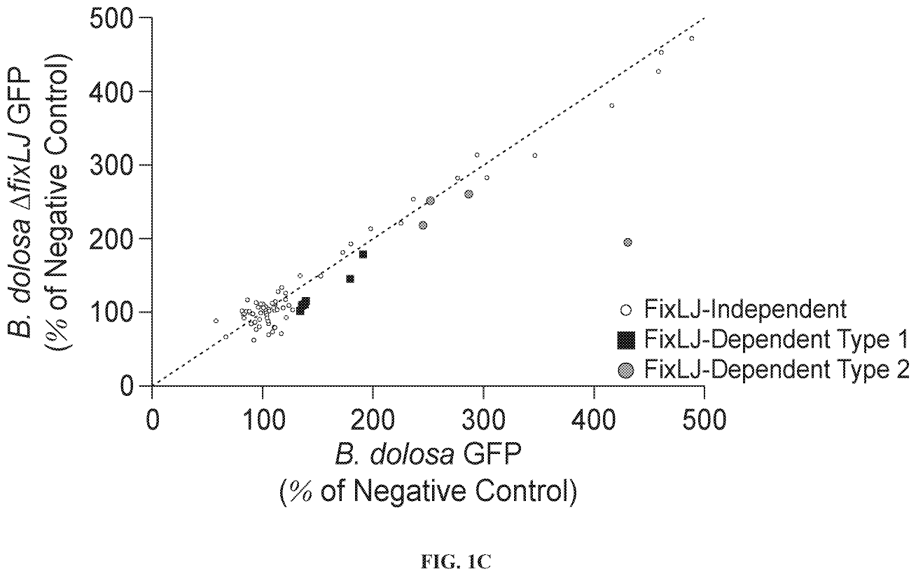

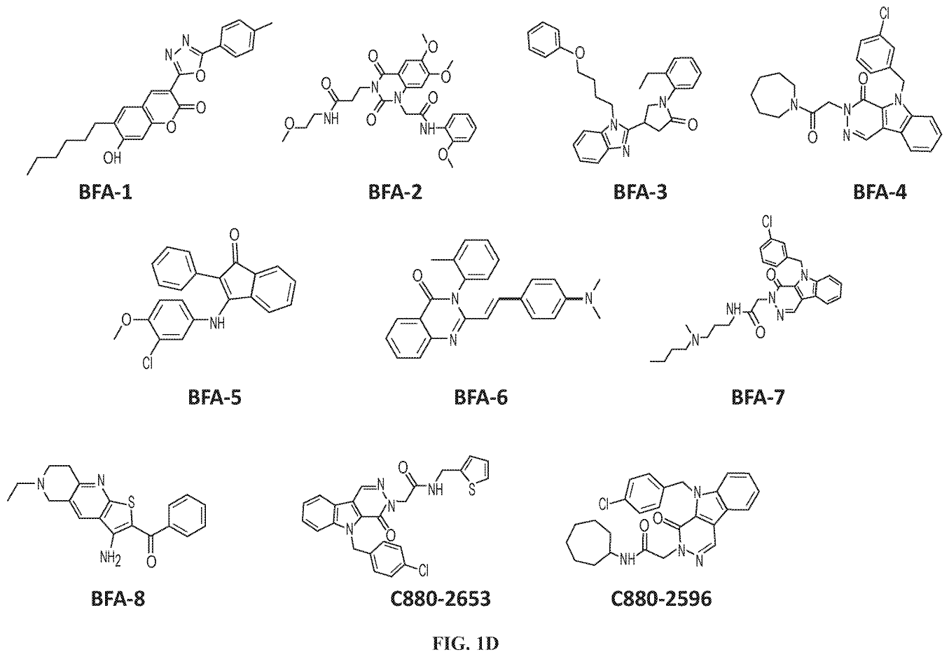

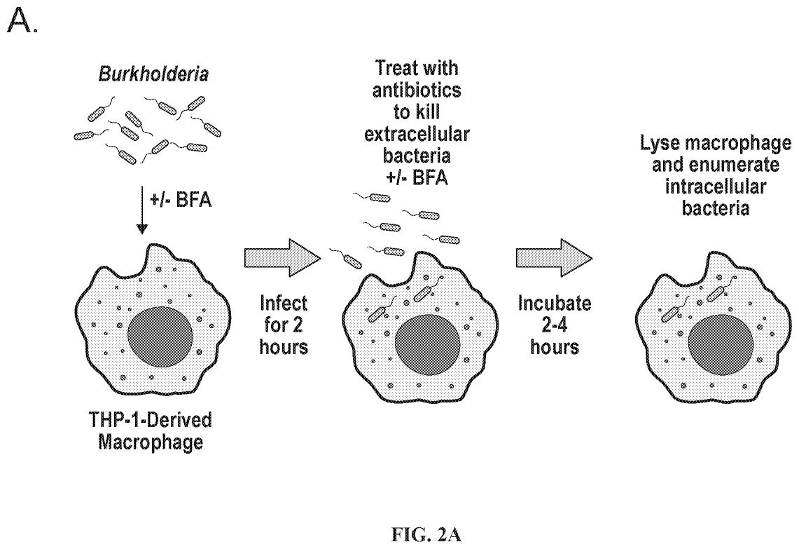

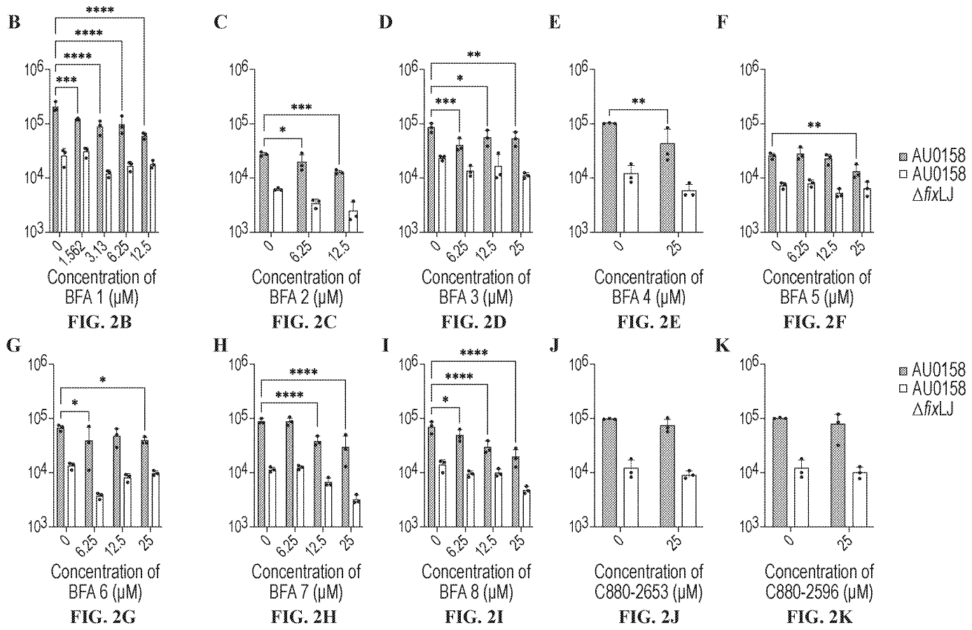

The ability of the 10 hits to inhibit B. dolosa invasion of and/or intracellular survival within macrophages were tested, which is a critical aspect of Burkholderia virulence (Valvano, M. A. Canadian Journal of Microbiology 61, 607-615 (2015); Schmerk, C. L. & Valvano, M. A. J Med Microbiol 62, 173-184 (2013); Mahenthiralingam, E., Urban, T. A. & Goldberg, J. B. Nat Rev Microbiol 3, 144-156 (2005)). Here, B. dolosa strain AU0158 was used, a CF clinical isolate that was also employed in the screen. In these assays, THP-1 cells, a human monocyte model, were differentiated into macrophage-like cells using phorbol 12-myristate-13-acetate (PMA) and infected with Burkholderia (5-10 bacteria per macrophage) for two hours while being exposed to compound or vehicle (DMSO). Cells were washed and then treated with kanamycin (to kill extracellular bacteria) along with compound or DMSO for an additional 2-4 hours. Cells were washed again, lysed, and then colony forming units (CFU) were determined by serial dilution and plating (FIG. 2A). Eight of the 10 compounds inhibited invasion/survival of B. dolosa in macrophages and were named Burkholderia Fix Activator (BFA) 1-8 (FIG. 2). BFA1 inhibited B. dolosa virulence at concentrations as low as the lowest tested dose of 1.5 μM (FIG. 2B) while the other 7 compounds inhibited virulence at concentrations between 6.25 and 12.5 μM (FIGS. 2C-2I). BFA compounds 2-8 inhibited the invasion/survival of B. dolosa up to 50% at the highest dose of compound tested. BFA1 was able to inhibit invasion/survival of B. dolosa in macrophages by ~75%. This reduction in the number of intracellular bacteria was fixLJ-specific, as the B. dolosa fixLJ deletion mutant, which already is less virulent than its parental strain, did not have significant reductions in intracellular bacteria when treated with any of the BFA compounds (FIG. 2, white bars). Two of the 10 hits were not able to inhibit the invasion/survival of B. dolosa in macrophages (FIGS. 2J and 2K) and were not further evaluated.

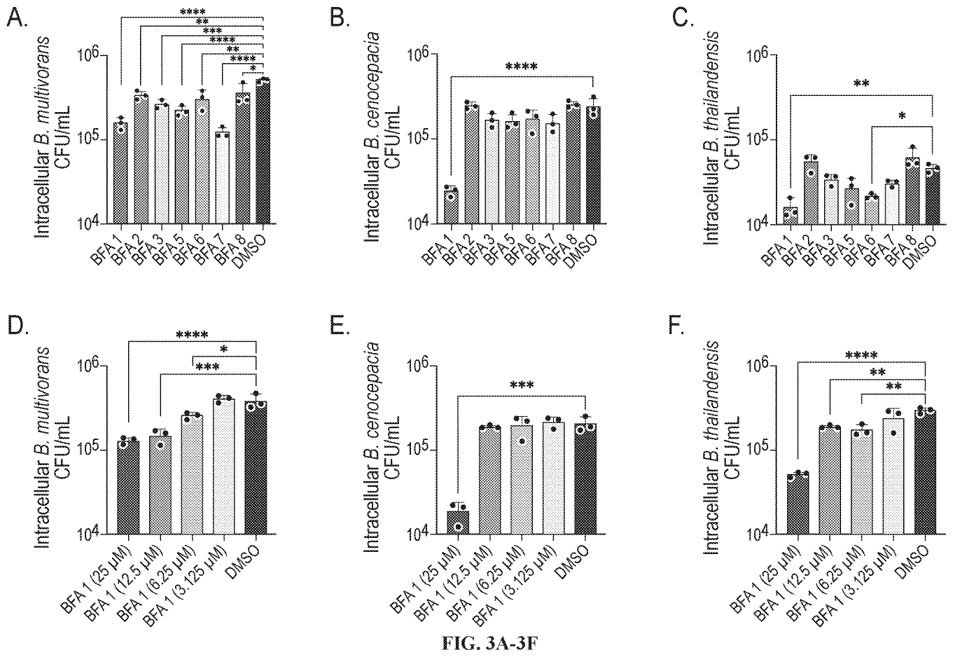

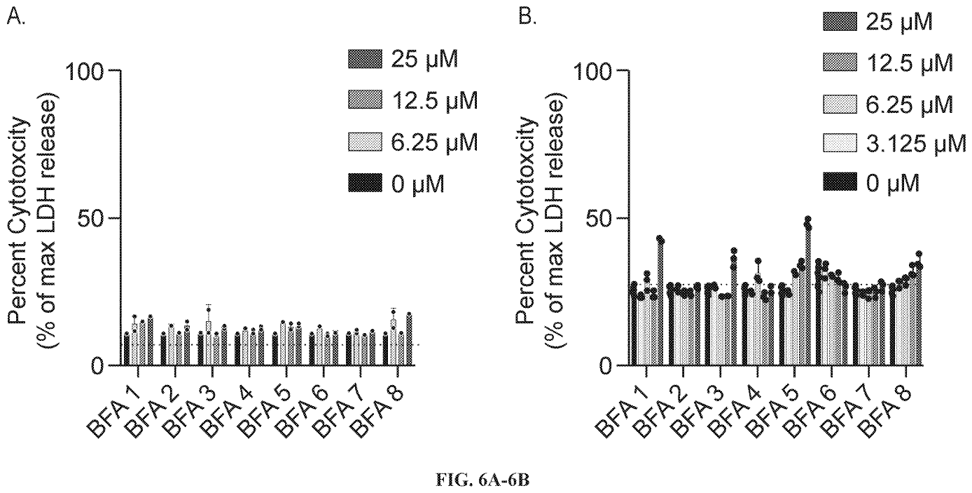

The cytotoxicity of BFA compounds was also measured by measuring lactate dehydrogenase (LDH) release from a human lung epithelial cell line (A549) and from THP-1 derived macrophages using commercially available kits. As shown in FIG. 6, it was found that none of the compounds caused significant cytotoxicity after overnight exposure at any of the tested concentrations. These findings demonstrate that BFA compounds can inhibit the virulence of Burkholderia in a fixLJ-specific mechanism without significant toxicity in vitro. In additional to measuring the ability of BFA compounds to inhibit the virulence of B. dolosa, the ability of select BFA compounds to inhibit the invasion of and/or intracellular survival of other pathogenic Burkholderia species was measured using the same THP-1-derived macrophage infection model. The ability of 7 of the 8 BFA compounds (25 μM) to inhibit invasion/intracellular survival of B. cenocepacia, B. multivorans, and B. thailandensis was measured. B. thailandensis is a model for B. pseudomallei that does not require BSL3 facilities (Kovacs-Simon, A. et al., BMC microbiology 19, 97 (2019)). BFA1 inhibited the invasion/survival of all three additional Burkholderia species (FIGS. 3A-3C). All seven of the tested BFA compounds inhibited the invasion/survival of B. multivorans (FIG. 3A). BFA1 was also able to inhibit the invasion/survival of B. cenocepacia (FIG. 3B) and B. thailandensis (FIG. 3C). BFA6 was also able to inhibit the invasion/survival of B. thailandensis (FIG. 3C), while there was a trend towards significance for other BFA compounds to inhibit B. thailandensis. Since BFA1 had the highest activity, multiple doses were evaluated for their ability to inhibit invasion/survival of other Burkholderia. Doses of 6.25 μM of BFA1 inhibited the invasion/survival of B. multivorans (FIG. 3D) and B. thailandensis (FIG. 3F), while higher doses were needed to inhibit B. cenocepacia (FIG. 3E).

### Example 3: In Silico Docking Studies Demonstrate BFA1 Interaction with ATP/ADP-Binding Pocket of FixL

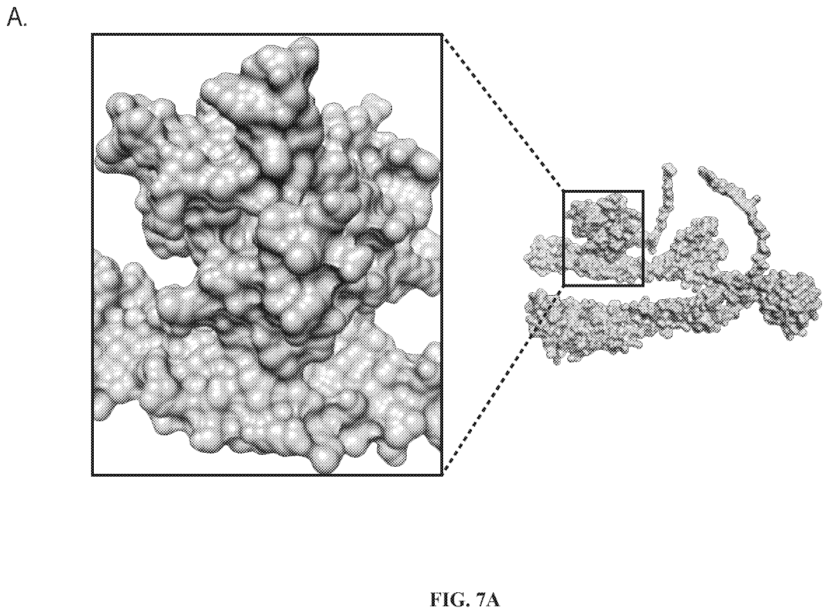

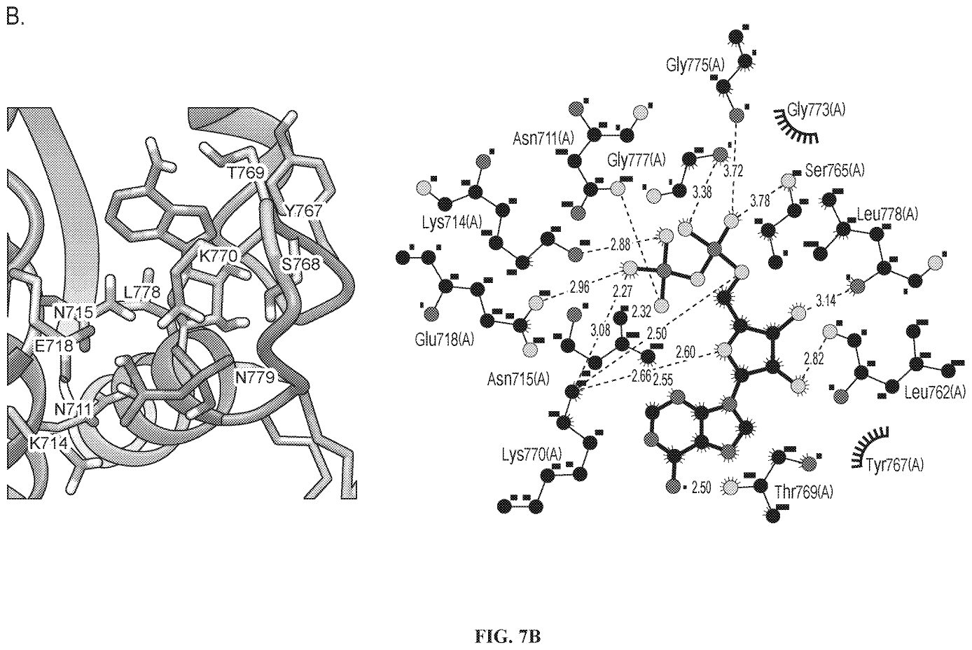

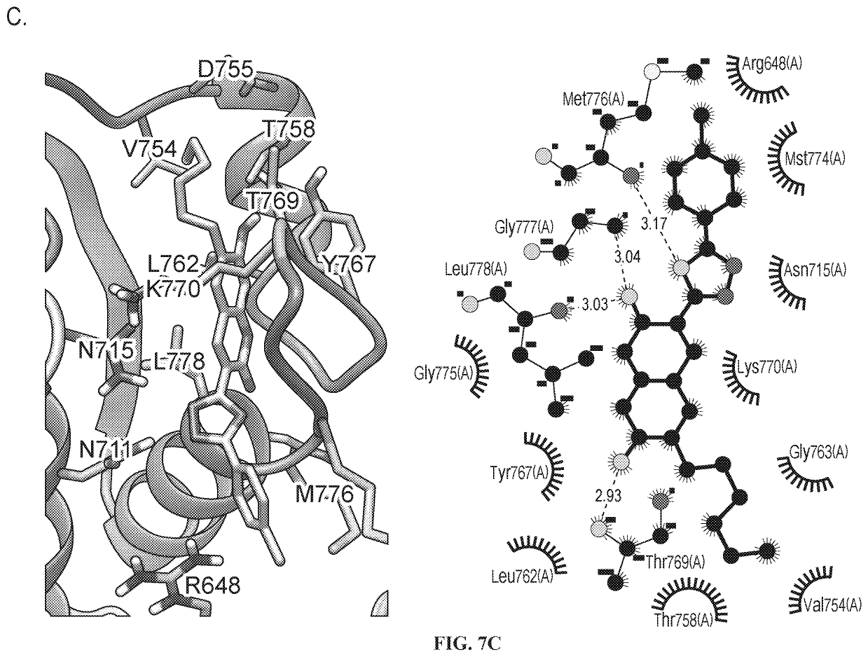

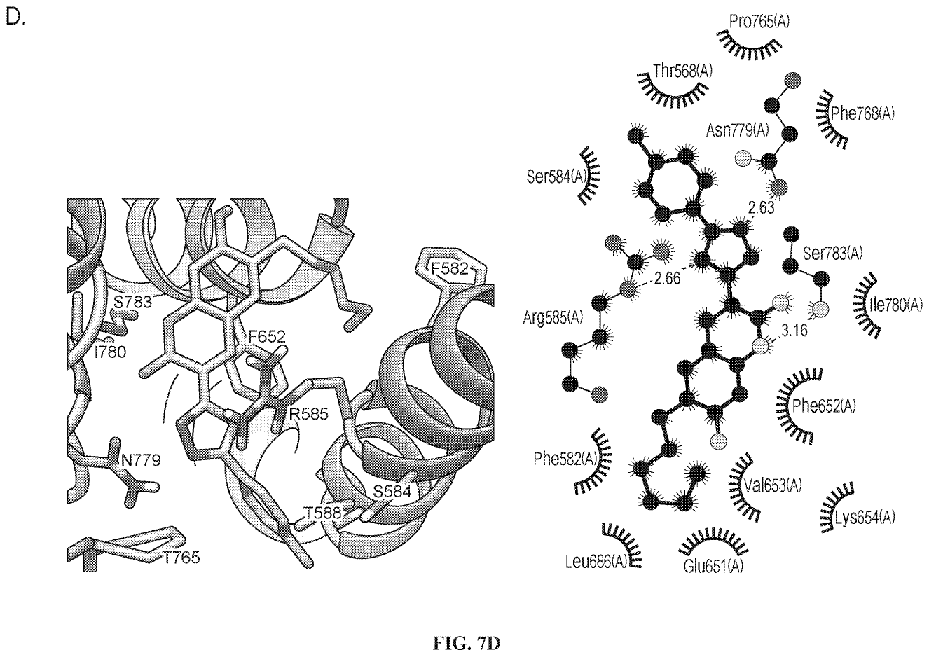

Docking studies were performed with B. dolosa FixL (AlphaFold ID: A0A0D5J096) using AutoDockFR in the flexible residue mode (Ravindranath, P. A. et al., PLOS Comput. Biol. 11, e1004586-e1004586 (2015)). Initial AutoSite calculations predicted two major potential ligand-binding sites on the protein (FIG. 7A, Table 2), of which the site with the highest affinity strongly resembled the ATP/ADP binding site of known histidine kinases, such as the structures from Caulobacter vibrioides (Protein Data Bank (PDB) ID: 5IDJ), Lactiplantibacillus plantarum (PDB ID: 4ZKI), or Thermotoga maritima (PDB ID: 6RH8). This site was thus chosen as the primary docking target, to which docking with ADP yielded a binding interaction similar to the mentioned histidine kinases, thereby validating the docking process (FIG. 7B).

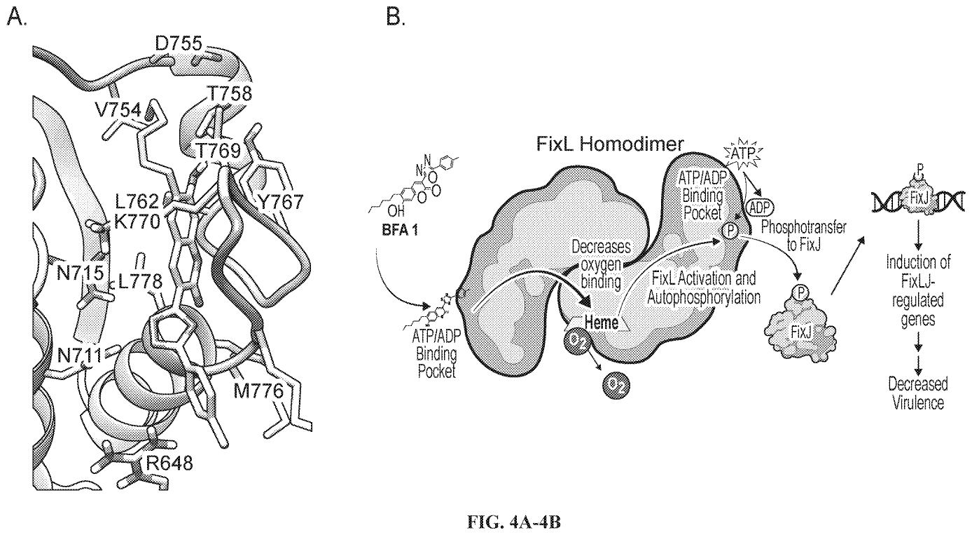

As expected, the predicted score for ADP binding to the second pocket was considerably lower. On the other hand, the binding scores for compound BFA1 were of similar magnitude for the two sites, in both cases better than for ADP (Table 6). For the ATP/ADP binding site, the docking analysis showed interactions between compound BFA1 and several residues in the binding pocket, with the coumarin oxygen atoms interacting with Gly777, Leu778, Thr769, and the oxadiazole oxygen interacting with Met776 (FIG. 4A, FIG. 7C). For the secondary binding site, the analysis revealed polar interactions between the ligand and residues Arg585, Asn779, and Ser783, as well as more lipophilic interactions with nonpolar residues (FIG. 7D). Docking simulations were also carried out using HADDOCK 2.4 (Honorato, R. V. et al. Front Mol Biosci 8, 729513 (2021); van Zundert, G. C. P. et al. Journal of Molecular Biology 428, 720-725 (2016)), revealing similar binding trends (Tables 6 and 7). A potential effect may also arise from binding to the second pocket, although the results in this case indicate a lower degree of more specific polar interactions compared to the ADP/ATP binding site.

A high-throughput screen was developed to identify small molecules that activate the Burkholderia FixLJ pathway, with the goal of inhibiting the virulence of these pathogens. The hypothesis was that a small molecule activating the FixLJ pathway could make Burkholderia less pathogenic, in a way coaxing the bacteria back to its soil existence where FixLJ activity is high.

The high-throughput screen identified 8 compounds that were denoted Burkholderia Fix Activators (BFA). These compounds previously had no known biological activity. All 8 of the BFA compounds inhibited the invasion and/or intracellular survival of B. dolosa in a fixLJ-specific manner (FIG. 2) but had no impact on bacterial growth in rich media. BFA1 inhibited virulence at a lower concentration and at a greater magnitude than the other BFA compounds. The BFA compounds caused no significant cytotoxicity, measured by LDH release, after 24-hour incubation, it is likely that macrophage death plays a negligible role in the BFA activity in antibiotic exclusion assays. Burkholderia are known for their extensive antibiotic resistance that is often mediated by decreases in outer membrane permeability and increases in efflux pump activity. Since clinical isolates of Burkholderia were used in the assays that identified the BFA compounds, it is clear that the BFA compounds are able to overcome these intrinsic drug penetration obstacles that have hindered antibiotic development.

The in silico predicted binding of BFA1 to the ATP/ADP binding pocket of FixL with a higher affinity than ADP is, on first review, counterintuitive for a molecule that increases FixL activity. However, for rhizobial FixL, ADP binding has been shown to decrease the affinity of FixL for oxygen, which results in activation of FixL (Nakamura, H., et al. Proc Natl Acad Sci USA 101, 2742-2746). This activation is related to the homodimer properties of FixL whereby ADP that is formed as part of the autophosphorylation of FixL binds to FixL, which decreases the binding affinity to oxygen in the other FixL molecule of the homodimer. This, in turn, allows for a positive feedback resulting in increased FixL activation. Without intending to be bound by theory, BFA1 binding to FixL at this same pocket is predicted to increase FixL activation as a result of decreased binding affinity for oxygen. These predictions are supported by findings that single amino acid changes of the asparagine residue (403) in the ADP-binding site of rhizobial FixL resulted in no change in oxygen affinity in response to ADP binding, demonstrating the importance of this residue in ADP binding. The homologous asparagine in the predicted ADP-binding site of Burkholderia FixL is at amino acid 715 and is predicted to interact with or be adjacent to BFA1 or ADP binding (FIGS. 4 and 7). The predicted mechanism of action is depicted in FIG. 4B, where BFA1 binds to FixL via the ATP/ADP binding pocket on the protein, which causes a decrease in binding affinity to oxygen and, in turn, activates FixL. FixL autophosphorylates, then transfers the phosphate group to the response regulator FixJ. Phosphorylated FixJ then binds to DNA and turns on transcription of target genes that are part of the FixLJ regulon resulting in a gene expression profile making the bacteria less virulent.

The method of using small-molecule activators of a pathway to inhibit the virulence of antibiotic-resistant pathogens is a novel approach for the development of new antibacterial therapies. By targeting bacterial virulence rather than bacterial growth, the emergence of resistance to therapies will be slower to occur. It is expected that resistance to BFA1 will be slow to develop since the amino acids of FixL predicted to be involved in binding to BFA1 are outside the FixL domains where mutations are seen during chronic infection. The results show that small-molecule activators of the Burkholderia FixLJ pathway are a promising new anti-virulence approach.

### Example 4: Activity of BFA Compounds Against Additional Bacterial Pathogens

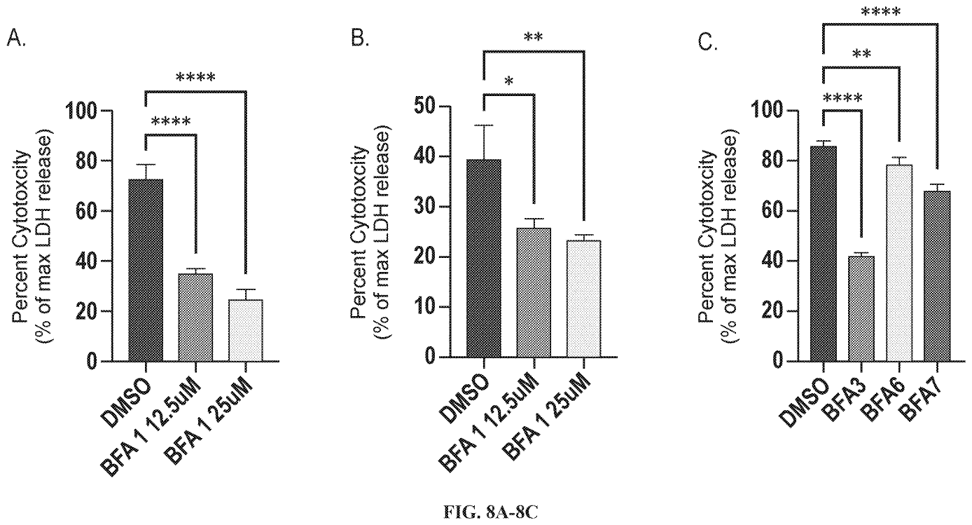

Selected BFA compounds were tested against other bacterial pathogens. While the fixLJ two-component system is restricted to Burkholderia and other non-pathogenic bacteria, BFA1 is likely to interact with FixL in a region that is conserved across many histidine kinases that are part of two-component systems. The ability of BFA1 to inhibit Pseudomonas aeruginosa-induced toxicity of A549 human lung epithelial cells was first tested. The model strains P. aeruginosa strain PA14 (exoU+) and P. aeruginosa strain PA01 (exoU−) were used, since PA14 contains the ExoU toxin, and this strain is more toxic than strain PA01. It was found that treatment with 12.5 or 25 μM of BFA was able to reduce cytotoxicity induced by P. aeruginosa strain PA14 by ~50 and 80%, respectively (FIG. 8A). It was also found that BFA1 was able to inhibit the cytotoxicity induced by P. aeruginosa strain PA01 (FIG. 8B). The overall level of cytotoxicity induced by this strain was lower than what was seen when cells were infected with PA14, consistent with PAO1 lacking exoU. Additional BFA compounds were also screened for their ability to inhibit PA140-induced cytotoxicity and it was found that BFA3, BFA6, and BFA7 were all able to significantly reduce cytotoxicity (FIG. 8C).

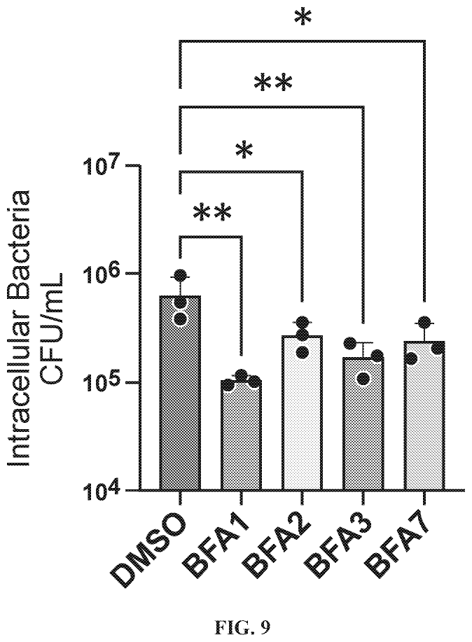

To determine whether BFA compounds could inhibit the virulence of Mycobacterium abscessus THP-1-derived macrophages were used. It was found that 25 μM BFA1, BFA2, BFA3, or BFA7 was able to inhibit the intracellular invasion/survival within the macrophages (FIG. 9).

TABLE 1
Number of hits from primary screen and number
of hits confirmed to be fixLJ-specific.

| Type of | # of Hits in | # of Confirmed |
|---|---|---|
| Hit | Primary Screen | fixLJ- Specific Hits |
| Strong | 15 | 3 |
| Medium | 11 | 4 |
| Weak | 57 | 4 |

TABLE 2
Potential ligand-binding pocket parameters.

| | | | Radius of | |
|---|---|---|---|---|
| Pocket | AutoSite score | Number of points | gyration | Buriedness |
| 1 | 43.69 | 347 | 5.71 | 0.85 |
| 2 | 39.25 | 301 | 5.18 | 0.82 |

TABLE 3
Strains used in the Examples

| | Notes |
|---|---|
| E. coli | |
| NEB 5-alpha Competent E. | DH5α derivative cloning strain |
| coli | |
| Burkholderia | |
| B. dolosa AU0158 | Clinical isolate |
| B. dolosa AU0158 ΔfixLJ | Clean, unmarked fixLJ deletion mutant |
| B. multivorans VC7102 | Clinical isolate |
| B. multivorans VC7102 Fix- | B. multivorans VC7102 + Fix-GFP |
| GFP Reporter | reporter |
| B. dolosa AU0158 Fix-GFP | B. dolosa AU0158 + Fix-GFP reporter |
| Reporter | integrated at attTn7 site downstream of |
| | AK34_4894 |
| B. dolosa AU0158 ΔfixLJ Fix- | B. dolosa ΔfixLJ + Fix-GFP reporter |
| GFP Reporter | integrated at attTn7 site downstream of |
| | AK34_4894 |
| B. thailandensis strain e264 | Isolate from rice field in Thailand, |
| | model for Burkholderia pseudomallei |
| B. cenocepacia K56-2 | Clinical Isolate |
| Pseudomonas | |
| Pseudomonas aeruginosa | |
| strain PA14 | |
| Pseudomonas aeruginosa | |
| strain PA01 | |
| Mycobacterium | |
| Mycobacterium abscessus | |
| strain ATCC19977 | |

TABLE 4
Plasmids used in the Examples

| | Notes |
|---|---|
| pTNS3 | Amp^{R}, helper plasmid for |
| | mini-Tn7 integration into |
| | attTn7 site |
| pRK2013 | Km^{R} conjugation helper |
| pUC18T-mini-Tn7T-Tp | Amp^{R}, Tp^{R} on mini-Tn7T; |
| | mobilizable |
| pfixK-reporter | pSCrhaB2 carrying B. |
| | dolosa fixK promoter -lacZ |
| | fusion, Tp^{R} |
| pIN301 | eGFP, CmR |
| Fix-GFP reporter | fixK promoter -GFP fusion |
| | in pUC18T-mini-Tn7T-Tp |

TABLE 5
Details of BFA compounds.

| BFA | Chem | | |
|---|---|---|---|
| # | Div # | Chemical Name | Smiles |
| BFA | 1564- | 6-HEXYL-7-HYDROXY-3-[5-(4- | CCCCCCc1cc2C═C(C(═O) |
| 1 | 0069 | METHYLPHENYL)-1,3,4-OXADIAZOL-2- | Oc2cc1O)c1nnc(c2ccc(C)cc |
| | | YL]-2H-CHROMEN-2-ONE | 2)o1 |
| BFA | C191- | 3-(6,7-DIMETHOXY-1-{[(2- | COCCNC(CCN1C(c2cc(c(c |
| 2 | 0406 | METHOXYPHENYL)CARBAMOYLMETH | c2N(CC(Nc2ccccc2OC)═O) |
| | | YL}-2,4-DIOXO-1,2,3,4- | C1═O)OC)OC)═O)═O |
| | | TETRAHYDROQUINAZOLIN-3-YL)-N-(2- | |
| | | METHOXYETHYL)PROPANAMIDE | |
| BFA | D011- | 1-(2-ETHYLPHENYL)-4-[1-(4- | CCc1ccccc1N1CC(CC1═O) |
| 3 | 1034 | PHENOXYBUTYL)-1H-1,3- | c1nc2ccccc2n1CCCCOc1cc |
| | | BENZODIAZOL-2-YL]PYRROLIDIN-2- | ccc1 |
| | | ONE | |
| BFA | C880- | 3-12-(AZEPAN-1-YL)-2-OXOETHYL]-5- | C1CCCN(CC1)C(CN1C(c2 |
| 4 | 2694 | [(3-CHLOROPHENYL)METHYL]-3H,4H, | c(C═N1)c1ccccc1n2Ce1ccc |
| | | 5H-PYRIDAZINO[4,5-B]INDOL-4-ONE | c(c1)[Cl])═O)═O |
| BFA | C301- | 3-[(3-CHLORO-4- | COc1ccc(cc1[Cl])NC1═C( |
| 5 | 5958 | METHOXYPHENYL)AMINO]-2-PHENYL- | C(c2ccccc12)═O)c1ccccc1 |
| | | 1H-INDEN-1-ONE | |
| BFA | 2764- | 2-[(1E)-2-[4- | Cc1ccccc1N1C(/C═C/c2ccc |
| 6 | 0017 | (DIMETHYLAMINO)PHENYL]ETHENYL]- | (cc2)N(C)C)═Nc2ccccc2C1 |
| | | 3-(2-METHYLPHENYL)-3,4- | ═O |
| | | DIHYDROQUINAZOLIN-4-ONE | |
| BFA | C880- | N-13- | CCCCN(C)CCCNC(CN1C( |
| 7 | 2827 | [BUTYL(METHYL)AMINO]PROPYL}-2- | c2c(C═N1)c1ccccc1n2Cc1c |
| | | {5-[(3-CHLOROPHENYL)METHYL]-4- | ccc(c1)[Cl])═O)═O |
| | | OXO-3H,4H,5H-PYRIDAZINO[4,5- | |
| | | B]INDOL-3-YL}ACETAMIDE | |
| BFA | C611- | 2-BENZOYL-6-ETHYL-5H,6H,7H,8H- | CCN1CCc2c(C1)cc1c(c(C( |
| 8 | 0649 | THIENO[2,3-B]1,6-NAPHTHYRIDIN-3- | c3ccccc3)═O)sc1n2)N |
| | | AMINE | |

TABLE 6
Ligand/protein clustering information predicted by ADFR

| Pock- | | Affinity | Clust. | Ref. | Clust. | RMSD | Energy |
|---|---|---|---|---|---|---|---|
| et | Ligand | (kcal/mol) | RMSD | RMSD | size | SD | SD |
| 1 | ADP | −9.4 | 0 | −1 | 21 | 0.5 | 0.7 |
| 2 | ADP | −6.9 | 0 | −1 | 5 | 0.7 | 0.3 |
| 1 | BFA1 | −12.6 | 0 | −1 | 4 | 0.8 | 0.4 |
| 2 | BFA1 | −12.4 | 0 | −1 | 11 | 0.6 | 0.5 |

TABLE 7
Ligand/protein clustering information predicted by HADDOCK.

| Pocket | Ligand | HADDOCK score | No. clusters |
|---|---|---|---|
| 1 | ADP | −82.708 | 64 |
| 2 | ADP | −55.213 | 112 |
| 1 | BFA1 | −48.647 | 16 |
| 2 | BFA1 | −46.693 | 76 |

### Methods

The results described herein above were obtained using the following methods and materials.

### Bacterial Strains, Plasmids, Cell Lines, and Growth Conditions

Bacterial strains used and generated in this study are listed in Table 3. For the generation of the reporter strain, BCC and E. coli were grown on LB plates or in LB medium and supplemented with following additives: ampicillin (100 μg/mL), chloramphenicol (20 μg/mL), trimethoprim (100 μg/mL for E. coli, 1 mg/mL for BCC), gentamicin (50 μg/mL). For some experiments, trypticase soy broth (TSB) or trypticase soy agar (TSA) was used for growth of Burkholderia. Human monocyte line THP-1 and human lung epithelial cell line A549 were obtained from ATCC and grown at 37 °C with 5% CO₂. THP-1 cells were cultured in RPMI-1640 medium containing 2 mM L-glutamine, 10 mM HEPES, 1 mM sodium pyruvate, 4500 mg/L glucose, and 1500 mg/L sodium bicarbonate, supplemented with 10% heat-inactivated fetal calf serum (FCS, Gibco) and 0.05 mM 2-mercaptoethanol. A459 cells were grown in RPMI 1640 with L-glutamine and 10% heat-inactivated fetal calf serum (FCS, Gibco). Penicillin and streptomycin were added for routine culture but were removed the day before and during experiments.

### Genetic Manipulations and Strain Construction

All plasmids used and generated in this study are listed in Table 4. To generate the GFP reporter of FixLJ pathway activity (Fix-GFP reporter), the first 23 bp of fixK and the immediate 243 bp upstream of the start codon from pfixK-reporter were cloned in-frame with eGFP gene from pIN301 into the multiple cloning site of pUC18-mini-Tn7-Tp. Use of a mini-Tn7 vector allows for stable chromosomally integration at an attTn7 site. The plasmid was transformed into NEB 5-alpha competent E. coli, and the sequence of the plasmid was confirmed using PCR and Sanger sequencing. The Fix-GFP reporter was conjugated into B. dolosa strain AU0158, the AU0158 fixLJ deletion mutant, and B. multivorans strain VC7102 with pRK2013 and pTNS3 using published procedures (Schaefers, M. M. et al., PLOS Pathog 13, e1006116 (2017)). Conjugants were selected for by plating on LB agar containing trimethoprim (1 mg/mL) and gentamicin (50 μg/mL). Insertions into the attTn7 site downstream of AK34_4894 was confirmed by PCR of B. dolosa strains as previously published (Schaefers, M. M. et al., PLOS Pathog 13, e1006116 (2017); Schaefers, M. M. et al., mBio 12, e01823-01821 (2021)).

### Small Molecule High Throughput Screens

Small-molecule screens were conducted at the Institute of Chemistry and Cell Biology (ICCB)-Longwood Screening Facility at Harvard Medical School. B. multivorans strain VC7102, with Fix-GFP reporter, was grown overnight in TSB at 37 °C with shaking and diluted 1:100 in fresh TSB the day of the assay. 30 μL per well was added into a black clear-bottom 384-well plate using Thermo Multidrop Combi pipette. 30 μL per well of TSB was also added into each well containing a test compound using the Combi pipette. Wells that served as negative controls contained 30 μL of TSB plus DMSO (0.078% v/v in TSB, final concentration 0.039%). For positive fluorescence control wells, 30 μL of TSB containing 78 μM of benserazide (final concentration 39 μM) was added. 300 nL solutions of each compound in DMSO were pin-transferred to each plate using an Epson Compound Transfer Robot from a compound library plate. For most compound library plates the stock concentration was 10 nM, but some plates had different concentrations typically 1-10 mM, details are posted in screen data deposited in PubChem. For every assay, 2 replicate assay plates were set up. Initial OD600 and GFP fluorescence were measured using PerkinElmer EnVision with Photometric 600 filter (600 nm, 8 nm bandpass), FITC 535 (Excitation filter FITC 585, Emission Filter FITC 535). Plates were stacked 5 high, covered with lids, and incubated at 37 °C overnight (~18 h). The following day, assay plates were read using the PerkinElmer EnVision as above. For each well, the initial GFP fluorescence intensity values were subtracted from overnight GFP fluorescence intensity values to calculate the AGFP for each well. The average AGFP was calculated by averaging the 2 replicate wells for each library well. The mean and standard deviation for AGFP of the negative control wells on the replicate plates was calculated. A compound was determined to be a strong hit if the average AGFP was more than 9 standard deviations above mean AGFP for the negative control wells on the 2 replicate plates. A compound was determined to be a moderate hit if the average AGFP was more than 6 standard deviations above mean AGFP for the negative control, and a compound was determined to be a weak hit if the average AGFP was more than 3 standard deviations above mean AGFP for the negative control for its respective plate. Subsequent analysis of data from the primary screen identified 3 additional hits that were not identified in first analysis and were not further evaluated. For “cherry-pick” studies, overnight cultures of B. multivorans strain VC7102, B. dolosa strain AU0158, and B. dolosa strain AU0158 fixLJ deletion mutant were diluted and plated in wells of a 384-well plate. 300 nL of selected compounds were plated into wells using a HP D300e liquid dispenser so that 2 wells of each bacterial strain were treated with compound. Plates were incubated, and the OD600 and GFP fluorescence intensity were measured as described above.

### Bacterial Invasion Assays

The ability of BFA compounds to inhibit the uptake of and/or intracellular survival of Burkholderia into THP-1-derived macrophages was determined using published protocols (Schaefers, M. M. et al., PLOS Pathog 13, e1006116 (2017); Schaefers, M. M. et al., mBio 12, e01823-01821 (2021)). Human THP-1 monocytes were differentiated into macrophages by seeding 1 mL into 24-well plates at 7.5×10⁵ cells/mL with 200 nM phorbol 12-myristate 13-acetate (PMA). Log-phase Burkholderia were grown and washed in RPMI containing 10% heat-inactivated FCS three times and diluted to ~2×10⁶ CFU/mL and mixed with BFA compounds or DMSO (vehicle control). 1 mL/well (MOI of ~10:1) of the bacterial suspension was used to infect THP-1 derived macrophages. Plates were spun at 500 g for 5 minutes to synchronize infection and then incubated for 2 hours at 37 °C with 5% CO₂. To determine the number of intracellular bacteria, separate infected wells were washed two times with PBS and then incubated with RPMI plus 10% heat-inactivated FCS containing BFA or DMSO (vehicle control) with kanamycin (1 mg/mL) or kanamycin plus ceftazidime (1 mg/mL each for B. cenocepacia) for 2-4 hours. Monolayers were washed three times with PBS, lysed with 1% Triton-X100, serially diluted, and plated to enumerate the number of bacteria.

### LDH Release Assay

A549 cells were grown to confluence in 96-well plates. Human THP-1 monocytes were differentiated into macrophages by 72-hour PMA treatment and seeded into 96-well plates at density of 7.5×10⁴ cells/well. A549 cells and THP-1-derived macrophages were then treated with BFA (0-25 μM in DMSO) for 24 hours. LDH release was measured within supernatants using CytoTox 96® Non-Radioactive Cytotoxicity Assay (Promega) per manufacture's protocol. For a positive control, untreated wells were incubated with 10× lysis buffer (provided with kit) during the last 30 minutes. Percent cytotoxicity was determined relative to maximum LDH release from cells treated with lysis buffer.

### In Silico Docking Studies

Docking studies were performed using AutoDockFR Suite 1.0 in the flexible residue mode (Ravindranath, P. A. et al., PLOS Comput. Biol. 11, e1004586-e1004586 (2015)).

Calculations were performed with 8 independent searches, each of which with 50 genetic algorithm evolutions associated with 2×10⁶ evaluations of the scoring function. Potential binding sites were identified by AutoSite 1.0. Residues Asn715, Tyr767, Ser768, Thr769, and Lys770 were set as flexible for pocket 1 and residues Arg585, Asn779, Ser783 for pocket 2. Docking simulations were also carried out using HADDOCK 2.4 (Honorato, R. V. et al., Front Mol Biosci 8, 729513 (2021); van Zundert, G. C. P. et al., Journal of Molecular Biology 428, 720-725 (2016)), using the default settings for small molecule-protein docking with RMSD-based clustering. The regions of the two binding sites identified by ADFR were investigated using corresponding active residues (Asn715, Tyr767, Ser768, Thr769, Lys770 at pocket 1; Arg585, Asn779, Ser783 at pocket 2). 1000 structures were generated initially and 200 clusters were screened out after refinement. All docking results were visualized by UCSF Chimera 1.17.3 and LigPlot+ 2.2 (Pettersen, E. F. et al., J Comput Chem 25, 1605-1612 (2004); Laskowski, R. A. & Swindells, M. B., Journal of Chemical Information and Modeling 51, 2778-2786 (2011)).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of reducing the virulence of a bacterial cell, the method comprising:
contacting a bacterial cell with an agent selected from the group consisting of
6-Hexyl-7-Hydroxy-3-[5-(4-Methylphenyl)-1,3,4-Oxadiazol-2-Yl]-2h-Chromen-2-One;
3-(6,7-Dimethoxy-1-{[(2-Methoxyphenyl)Carbamoyl]Methyl}-2,4-Dioxo-1,2,3,4-Tetrahydroquinazolin-3-Yl)-N-(2-Methoxyethyl)Propanamide;
1-(2-Ethylphenyl)-4-[1-(4-Phenoxybutyl)-1h-1,3-Benzodiazol-2-Yl]Pyrrolidin-2-One;
3-[2-(Azepan-1-Yl)-2-Oxoethyl]-5-[(3-Chlorophenyl)Methyl]-3h,4h,5h-Pyridazino[4,5-B]Indol-4-One;
3-[(3-Chloro-4-Methoxyphenyl)Amino]-2-Phenyl-1h-Inden-1-One;
2-[(1e)-2-[4-(Dimethylamino)Phenyl]Ethenyl]-3-(2-Methylphenyl)-3,4-Dihydroquinazolin-4-One;
N-{3-[Butyl(Methyl)Amino]Propyl}-2-{5-[(3-Chlorophenyl)Methyl]-4-Oxo-3h,4h,5h-Pyridazino[4,5-B]Indol-3-Yl}Acetamide;
2-Benzoyl-6-Ethyl-5h,6h,7h,8 h-Thieno[2,3-B]1,6-Naphthyridin-3-Amine, wherein the method reduces the virulence of the bacterial cell.

2. The method of claim 1, wherein the agent inhibits the invasion of, uptake of, and/or intracellular survival of the bacteria.

3. The method of claim 2, wherein the bacteria is a Burkholderia, Pseudomonas, or Mycobacterium.

4. A method of altering activity of a two-component system in a bacterial cell, the method comprising:
contacting the bacterial cell with an agent selected from the group consisting of
6-Hexyl-7-Hydroxy-3-[5-(4-Methylphenyl)-1,3,4-Oxadiazol-2-Yl]-2h-Chromen-2-One;
3-(6,7-Dimethoxy-1-{[(2-Methoxyphenyl)Carbamoyl]Methyl}-2,4-Dioxo-1,2,3,4-Tetrahydroquinazolin-3-Yl)-N-(2-Methoxyethyl)Propanamide;
1-(2-Ethylphenyl)-4-[1-(4-Phenoxybutyl)-1h-1,3-Benzodiazol-2-Yl]Pyrrolidin-2-One;
3-[2-(Azepan-1-Yl)-2-Oxoethyl]-5-[(3-Chlorophenyl)Methyl]-3h,4h,5h-Pyridazino[4,5-B]Indol-4-One;
3-[(3-Chloro-4-Methoxyphenyl)Amino]-2-Phenyl-1h-Inden-1-One;
2-[(1e)-2-[4-(Dimethylamino)Phenyl]Ethenyl]-3-(2-Methylphenyl)-3,4-Dihydroquinazolin-4-One;
N-{3-[Butyl(Methyl)Amino]Propyl}-2-{5-[(3-Chlorophenyl)Methyl]-4-Oxo-3h,4h,5h-Pyridazino[4,5-B]Indol-3-Yl}Acetamide;
2-Benzoyl-6-Ethyl-5h,6h,7h,8 h-Thieno[2,3-B]1,6-Naphthyridin-3-Amine, thereby altering activity of the two-component system in the bacterial cell.

5. The method of claim 1, wherein the two-component system comprises a histidine kinase.

6. The method of claim 4, wherein the two-component system regulates microaerobiosis in the bacterial cell or functions in a nitrogen fixation and/or a respiration pathway in the bacterial cell.

7. The method of claim 4, wherein the two-component system is a FixLJ system.

8. A method of reducing cell death in response to bacterial infection, the method comprising:
contacting a bacterial cell with an agent selected from the group consisting of
6-Hexyl-7-Hydroxy-3-[5-(4-Methylphenyl)-1,3,4-Oxadiazol-2-Yl]-2h-Chromen-2-One;
3-(6,7-Dimethoxy-1-{[(2-Methoxyphenyl)Carbamoyl]Methyl}-2,4-Dioxo-1,2,3,4-Tetrahydroquinazolin-3-Yl)-N-(2-Methoxyethyl)Propanamide;
1-(2-Ethylphenyl)-4-[1-(4-Phenoxybutyl)-1h-1,3-Benzodiazol-2-Yl]Pyrrolidin-2-One;
3-[2-(Azepan-1-Yl)-2-Oxoethyl]-5-[(3-Chlorophenyl)Methyl]-3h,4h,5h-Pyridazino[4,5-B]Indol-4-One;
3-[(3-Chloro-4-Methoxyphenyl)Amino]-2-Phenyl-1h-Inden-1-One;
2-[(1e)-2-[4-(Dimethylamino)Phenyl]Ethenyl]-3-(2-Methylphenyl)-3,4-Dihydroquinazolin-4-One;
N-{3-[Butyl(Methyl)Amino]Propyl}-2-{5-[(3-Chlorophenyl)Methyl]-4-Oxo-3h,4h,5h-Pyridazino[4,5-B]Indol-3-Yl}Acetamide;
2-Benzoyl-6-Ethyl-5h,6h,7h,8 h-Thieno[2,3-B]1,6-Naphthyridin-3-Amine, wherein the method reduces cell death in response to infection by the bacterial cell.

9. A method of reducing the virulence of a Burkholderia bacterium, the method comprising contacting the Burkholderia bacterium with 6-Hexyl-7-Hydroxy-3-[5-(4-Methylphenyl)-1,3,4-Oxadiazol-2-Yl]-2h-Chromen-2-One, thereby reducing the virulence of the Burkholderia bacterium.

10. The method of claim 9, wherein the method reduces macrophage invasion by the bacterium, macrophage uptake of the bacterium, and/or intracellular survival by the bacterium.

11. The method of claim 1, wherein the bacterial cell is a member of the Burkholderia, Pseudomonas or Mycobacterium genus.

12. The method of claim 11, wherein the Burkholderia is B. dolosa, B. multivorans, B. thailandensis, or B. cenocepacia.

13. A method of treating a subject having a bacterial infection, the method comprising:
administering to the subject
an agent selected from the group consisting of
6-Hexyl-7-Hydroxy-3-[5-(4-Methylphenyl)-1,3,4-Oxadiazol-2-Yl]-2h-Chromen-2-One;
3-(6,7-Dimethoxy-1-{[(2-Methoxyphenyl)Carbamoyl]Methyl}-2,4-Dioxo-1,2,3,4-Tetrahydroquinazolin-3-Yl)-N-(2-Methoxyethyl)Propanamide;
1-(2-Ethylphenyl)-4-[1-(4-Phenoxybutyl)-1h-1,3-Benzodiazol-2-Yl]Pyrrolidin-2-One;
3-[2-(Azepan-1-Yl)-2-Oxoethyl]-5-[(3-Chlorophenyl)Methyl]-3h,4h,5h-Pyridazino[4,5-B]Indol-4-One;
3-[(3-Chloro-4-Methoxyphenyl)Amino]-2-Phenyl-1h-Inden-1-One;
2-[(1e)-2-[4-(Dimethylamino)Phenyl]Ethenyl]-3-(2-Methylphenyl)-3,4-Dihydroquinazolin-4-One;
N-{3-[Butyl(Methyl)Amino]Propyl}-2-{5-[(3-Chlorophenyl)Methyl]-4-Oxo-3h,4h,5h-Pyridazino[4,5-B]Indol-3-Yl}Acetamide;
2-Benzoyl-6-Ethyl-5h,6h,7h,8 h-Thieno[2,3-B]1,6-Naphthyridin-3-Amine, thereby treating the infection.

14. The method of claim 13, wherein the method reduces the ability of the Burkholderia bacterium to invade an immune cell, reduces uptake of the Burkholderia bacterium by an immune cell, and/or reduces survival of the Burkholderia bacterium within an immune cell.

15. The method of claim 13, wherein the immune cell is a macrophage.

16. The method of claim 15, wherein the subject has cystic fibrosis or chronic granulomatous disease.

17. A method of treating a Burkholderia infection in a subject having cystic fibrosis or chronic granulomatous disease, the method comprising administering to the subject 6-Hexyl-7-Hydroxy-3-[5-(4-Methylphenyl)-1,3,4-Oxadiazol-2-Yl]-2h-Chromen-2-One, thereby treating the infection.

18. The method of claim 1, wherein the agent is 6-HEXYL-7-HYDROXY-3-[5-(4-METHYLPHENYL)-1,3,4-OXADIAZOL-2-YL]-2H-CHROMEN-2-ONE.

19. A kit for reducing or inhibiting the virulence of a bacterial cell, the kit comprising an agent selected from the group consisting of 6-Hexyl-7-Hydroxy-3-[5-(4-Methylphenyl)-1,3,4-Oxadiazol-2-Yl]-2h-Chromen-2-One;
3-(6,7-Dimethoxy-1-{[(2-Methoxyphenyl)Carbamoyl]Methyl}-2,4-Dioxo-1,2,3,4-Tetrahydroquinazolin-3-Yl)-N-(2-Methoxyethyl)Propanamide;
1-(2-Ethylphenyl)-4-[1-(4-Phenoxybutyl)-1h-1,3-Benzodiazol-2-Yl]Pyrrolidin-2-One;
3-[2-(Azepan-1-Yl)-2-Oxoethyl]-5-[(3-Chlorophenyl)Methyl]-3h,4h,5h-Pyridazino[4,5-B]Indol-4-One;
3-[(3-Chloro-4-Methoxyphenyl)Amino]-2-Phenyl-1h-Inden-1-One;
2-[(1e)-2-[4-(Dimethylamino)Phenyl]Ethenyl]-3-(2-Methylphenyl)-3,4-Dihydroquinazolin-4-One;
N-{3-[Butyl(Methyl)Amino]Propyl}-2-{5-[(3-Chlorophenyl)Methyl]-4-Oxo-3h,4h,5h-Pyridazino[4,5-B]Indol-3-Yl}Acetamide;
2-Benzoyl-6-Ethyl-5h,6h,7h,8 h-Thieno[2,3-B]1,6-Naphthyridin-3-Amine, and directions for administering the agent to treat a bacterial infection.

20. The kit of claim 19, wherein the agent is 6-Hexyl-7-Hydroxy-3-[5-(4-Methylphenyl)-1,3,4-Oxadiazol-2-Yl]-2h-Chromen-2-One.


SEQUENCE LISTING (39 sequences)

SEQ ID NO: 1 - protein, 838 aa, Burkholderia dolosa
MLTDRLFARSARPSGPPAESQPSRWHHGPWWSNSYLLTPLLSILVFLVVMSLILWSLNRREQQQQEDTLFRNVAWAQQQIRLSMTGAQEQLQALSRDLASGRLDQNAFQMAVADVMQTHPEILYLNWYTAPGVQRWPSVHPPLLGQRLAKPGDAQLQEAVRGAYDEARSTRRQAYSPLIYDDFGNGFITLQTPVMRGDREYLGSIAAVFSVEGILKHDIPQELSSKYKISITDANNRELSSTSTRPRLPRDSHYDLPLDPPGQGLTVRVYAFPQLTNLTNNTLVWLVAGLSCFVLWSLWSLWKHTRQRFEAQQALYAEAFFRRAMENSVLIGMRVLDMHGRITHVNPAFCRMTGWDESDLVGKVAPFPYWPRDAYPEMQRQLDMTLRGKAPSSGFELRVRRKNGTLFHARLYVSPLIDSSGRQTGSMSSMTDITEPKRAREELAAAHERFTTVLESLDAAVSVLAADEAELLFANRYYRHLFGIRPDGHLELSGGGFDRAQASSDSIDMVDAFAGLPAAALTNSTADAQEVYVESIQKWFEVRRQYIQWVDGHLAQMQIATDITTRKKAQELAHQQEEKLQFTSRLMTMGEMASSIAHELNQPLAAINNYCSGTLALVKSGRATPETLQPALEKTAQQALRAGMIVKRIREFVKRSEPKRQPARVADIVADAVGLAEIEARKRRIRIVTEILARMPIIYVDPVLIEQVLVNLMKNAAEAMADVKPASADGVIRVVADIDAGFVDIRVIDQGPGVDEATAERLFEPFYSTKSDGMGMGLNICRSIIESHRGRLWVVNNVEPDGRISGATFHCSLPIGEPADLGRGGSEASASHTVTGEL

SEQ ID NO: 2 - protein, 838 aa, Burkholderia multivorans
MLTDRLFARSARPSGPPAESQPSRWHHGPWWSNSYLLTPLLSILVFLVVMSLILWSLNRREQQQQEDTLFRNVAWAQQQIRLSMTGAQEQLQALSRDLASGRLDQNAFQMAVADVMQTHPEILYLNWYTAPGVQRWPTVHPPLLGQRLAKPGDAQLQDAVRGAYDEARSSRRQAYSPLIYDDFGNGYITLQTPVMRGDRDYLGSLAAVFSVEGILKHDIPQELSSKYKISITDANNRELSSTSTRPRLPRDSHYDLPLDPPGQGLTVRVYAFPQLTNLTNNTLVWLVAGLSCFVLWSLWSLWKHTRQRFEAQQALYAEAFFRRAMENSVLIGMRVLDMHGRITHVNPAFCRMMGWDESDLVGKVAPFPYWPRDAYPEMQRQLDMTLRGKAPSSGFELRVRRKNGTLFHARLYVSPLIDSSGRQTGWMSSMTDITEPKRAREELAAAHERFTTVLESLDAAVSVLAADEAELLFANRYYRHLFGIRPDGHLELSGGGFDRAQASSDSIDMVDAFAGLPATALTNTAADAQEVYVESIQKWFEVRRQYIQWVDGHLAQMQIATDITTRKKAQELAHQQEEKLQFTSRLMTMGEMASSIAHELNQPLAAINNYCSGTLALVKSGRGTPEMLQPALEKTAQQALRAGMIVKRIREFVKRSEPKRQPARVADIVADAVGLAEIEARKRRIRIVTEILARMPIIYVDPVLIEQVLVNLMKNAAEAMADVKPPSADGVIRVVADIDAGFVDIRVIDQGPGVDEATAERLFEPFYSTKSDGMGMGLNICRSIIESHRGRLWVVNNVEPDGRISGATFHCSLPIGEPADLGQGGSETSASHTVTGEL

SEQ ID NO: 3 - protein, 846 aa, Burkholderia pseudomallei
MQDALSYNPAMLTDRLFARSARPSGSPAESQPSRWHHGPWWSNSYLLTPLLSILVFLVVMSLILWSLNRREEQQQEDTLFRNVAWAQQQIRLSMTSAQEQLQAFSRDIAAGRIDEHAFQATVGDVMQAHPEILYLNWYTSPGTKRWPTMQLPLLGQRLAKPNDAQMDEVVRGAYAQARGTRRQSYSPLVYDDFGNGYLTLQTPVIREREYLGSIAAVFSVEGILKHDIPPELSAKYKISITDANNRELASTSSRPRLPRDAHYDLPLDPPGQGLTVRVYAYPQTTNLTNNTLVWLVAGLSCFVLWSLWSLWKHTRQRFEAQQALYAEAFFRRAMENSVLIGMRVLDMHGRITHVNPAFCRMTGWDESDLVGKTAPFPYWPRDAYPEMQRQLDMTLRGKAPSSGFELRVRRKDGSLFHARLYVSPLIDSAGRQTGWMSSMTDITEHKRAREELAAAHERFTTVLESLDAAVSVLAADEAELLFANRYYRHLFGIRPDGHLELSGGGFDTAQASSDSIDMVDAYAGLPAAALTESTADAQEVYVESIQKWFEVRRQYIQWVDGHLAQMQIATDITTRKKAQELAHQQEEKLQFTSRLMTMGEMASSIAHELNQPLAAINNYCSGTLALVKSGRASPETLAPALEKTAQQALRAGMIVKRIREFVKRSEPKRQPSRVADIVADAVGLAEIEARKRRIRIVTEIRARMPIIYVDPVLIEQVLVNLMKNAAEAMQEARPQAENGVIRVVADLEAGFVDIRVIDQGPGVDEATAERLFEPFYSTKSDGMGMGLNICRSIIESHRGRLWVVNNVEPDGLVSGATFHCSLPIGEPEDLGRGSETSPSQTVTGEI

SEQ ID NO: 4 - protein, 838 aa, Burkholderia contaminans
MLTDRLFARSARPSGPPAESQPSRWHHGPWWSNSYLLTPLLSILVFLVVMSLILWSLNRREQQQQEDTLFRNVAWAQQQIRLSMTGAQEQLQAVSRDLASGRLDQNAFQMAVADVMQTHPEILYLNWYTSPGVQRWPTVHPPLLGQRLAKPGDAQMQDAVRGAYDEARSTRRQAYSPLIYDDFGNGFITLQTPVMRGDREYLGSIAAVFSVEGILKHDIPQELSSKYKISITDANNRELSSTSTRPRLPRDSHYDLPLDPPGQGLTVRVYAFPQLTNLTNNTLVWLVAGLSCFVLWSLWSLWKHTRQRFEAQQALYAEAFFRRAMENSVLIGMRVLDMHGRITHVNPAFCRMTGWDETDLVGKVAPFPYWPRDAYPEMQRQLDMTLRGKAPSSGFELRVRRKNGTLFHARLYVSPLIDSSGRQTGWMSSMTDITEPKRAREELAAAHERFTTVLESLDAAVSVLAADEAELLFANRYYRHLFGIRPDGHLELSGGGFDRAQASSDSIDMVDAFAGLPAAALTSSTADAQEVYVESIQKWFEVRRQYIQWVDGHLAQMQIATDITTRKKAQELAHQQEEKLQFTSRLMTMGEMASSIAHELNQPLAAINNYCSGTLALVKSGRGTPETLQPALEKTAQQALRAGMIVKRIREFVKRSEPKRQPARVADIVADAVGLAEIEARKRRIRIVTEILARMPIIYVDPVLIEQVLVNLMKNAAEAMADVKPASVDGVIRVVADIEAGFVDIRVIDQGPGVDEATAERLFEPFYSTKSDGMGMGLNICRSIIESHRGRLWVVNNVEPDGRISGATFHCSLPIGEPADLGQGGREASASHTVTGEL

SEQ ID NO: 5 - protein, 838 aa, unidentified
MLTDRLFARSARPPGPPAGSQPSRWHHGPWWSNSYLLTPLLSILVFLVVMSLILWSLNRREQQQQEDTLFRNVAWAQQQIRLSMTGAQEQLQALSRDLASGRLDQNAFQMAVADVMQTHPEILYLNWYTAPGVQRWPTVHPPLLGQRLAKPGDAQMQDAVRGAYDEARSTRRQAYSPLIYDDFGNGFITLQTPVMRGDREYLGSIAAVFSVEGILKHDIPQELSSKYKISITDSNNRELSSTSTRPRLPRDSHYDLPLDPPGQGLTVRVYAFPQLTNLTNNTLVWLVAGLSCFVLWSLWSLWKHTRQRFEAQQALYAEAFFRRAMENSVLIGMRVLDMHGRITHVNPAFCRMTGWDETDLVGKVAPFPYWPRDAYPEMQRQLDMTLRGKAPSSGFELRVRRKNGTLFHARLYVSPLIDSSGRQTGWMSSMTDITEPKRAREELAAAHERFTTVLESLDAAVSVLAADEAELLFANRYYRHLFGIRPDGHLELSGGGFDRAQASSDSIDMVDAFAGLPAAALTSSTADAQEVYVESIQKWFEVRRQYIQWVDGHLAQMQIATDITTRKKAQELAHQQEEKLQFTSRLMTMGEMASSIAHELNQPLAAINNYCSGTLALVKSGRGTPETLQPALEKTAQQALRAGMIVKRIREFVKRSEPKRQPARVADIVADAVGLAEIEARKRRIRIVTEILARMPIIYVDPVLIEQVLMNLMKNAAEAMADVKPASADGVIRVVADIDAGFVDIRVIDQGPGVDEATAERLFEPFYSTKSDGMGMGLNICRSIIESHRGRLWVVNNVEPDGRISGATFHCSLPIGEPADLGQGGREASASHTVTGEL

SEQ ID NO: 6 - protein, 838 aa, unidentified
MLTDRLFARSARPSGPPAESQPSRWHHGPWWSNSYLLTPLLSILVFLVVMSLILWSLNRREQQQQEDTLFRNVAWAQQQIRLSMTGAQEQLQALSRDLASGRLDQNGFQMAVADVMQTHPEILYLNWYTSPGTQRWPTVHPPLLGQRLARPGDAQMQDAVRGAYDEARNSRRQAYAPLVYDDFGNGYITLQTPVMRGDREYLGSLAAVFSVEGILKHDIPQELSSKYKISITDANNRELSSTSSRPRLPRDSHYDLPLDPPGQGLTVRVYAFPQLTNLTNNTLVWLVAGLSCFVLWSLWSLWKHTRQRFEAQQALYAEAFFRRAMENSVLIGMRVLDMHGRITHVNPAFCRMTGWDESDLVGKVAPFPYWPRDSYPEMQRQLDMTLRGKAPSSGFELRVRRKNGTLFHARLYVSPLIDSAGRQTGWMSSMTDITEPKRAREELAAAHERFTTVLESLDAAVSVLAADEAELLFANRYYRHLFGIRPDGHLELSGGGFDRAQASSDSIDMVDTFAGLPAAALTGSTADAQEVYVESIQKWFEVRRQYIQWVDGHLAQMQIATDITTRKKAQELAHQQEEKLQFTSRLMTMGEMASSIAHELNQPLAAINNYCSGTLAMVKSGRATTETLAPALEKTAQQALRAGMIVKRIREFVKRSEPKRQPARVADIVADAVGLAEIEARKRRIRIVTEILARMPIIYVDPVLIEQVLVNLMKNAAEAMADVKPASADGVIRVIADLEAGFVDIRVIDQGPGVDEATAERLFEPFYSTKSDGMGMGLNICRSIIESHRGRLWVVNNVEADGRISGATFHCSLPIGEPADLARGGAEASASHTVTGEL

SEQ ID NO: 7 - protein, 838 aa, unidentified
MLTDRLFARSARPSGPPAESQPSRWHHGPWWSNSYLLTPLLSILVFLVVMSLILWSLNRREQQQQEDTLFRNVAWAQQQIRLSMTGAQEQLQALSRDLASGRLDQNAFQMAVADVMQTHPEILYLNWYTAPGVQRWPTVHPPLLGQRLAKPGDAQMQDAVRGAYDEARSTRRQAYSPLIYDDFGNGFITLQTPVMRGDREYLGSIAAVFSVEGILKHDIPQELSSKYKISITDANNRELSSTSTRPRLPRDSHYDLPLDPPGQGLTVRVYAFPQLTNLTNNTLVWLVAGLSCFVLWSLWSLWKHTRQRFEAQQALYAEAFFRRAMENSVLIGMRVLDMHGRITHVNPAFCRMTGWDESDLVGKVAPFPYWPRDAYPEMQRQLDMTLRGKAPSSGFELRVRRKNGTLFHARLYVSPLIDSSGRQTGWMSSMTDITEPKRAREELAAAHERFTTVLESLDAAVSVLAADEAELLFANRYYRHLFGIRPDGHLELSGGGFDRAQASSDSIDMVDAFAGLPAAALTSSTADAQEVYVESIQKWFEVRRQYIQWVDGHLAQMQIATDITTRKKAQELAHQQEEKLQFTSRLMTMGEMASSIAHELNQPLAAINNYCSGTLALVKSGRGTPETLQPALEKTAQQALRAGMIVKRIREFVKRSEPKRQPARVADIVADAVGLAEIEARKRRIRIVTEILARMPIIYVDPVLIEQVLMNLMKNAAEAMADVKPASADGVIRVVADIDAGFVDIRVIDQGPGVDEATAERLFEPFYSTKSDGMGMGLNICRSIIESHRGRLWVVNNVEPDGRISGATFHCSLPIGEPADLGQGGREASASHTVTGEL

SEQ ID NO: 8 - protein, 838 aa, unidentified
MLTDRLFARSARPSGPPAESQPSRWHHGPWWSNSYLLTPLLSILVFLVVMSLILWSLNRREQQQQEDTLFRNVAWAQQQIRLSMTGAQEQLQALSRDLASGRLDQNAFQMAVADVMQTHPEILYLNWYTAPGVQRWPTVHPPLLGQRLAKPGDAQLQDAVRGAYDEARSSRRQAYSPLIYDDFGNGYITLQTPVMRGDRDYLGSIAAVFSVEGILKHDIPQELSSKYKISITDANNRELSSTSTRPRLPRDSHYDLPLDPPGQGLTVRVYAFPQLTNLTNNTLVWLVAGLSCFVLWSLWSLWKHTRQRFEAQQALYAEAFFRRAMENSVLIGMRVLDMHGRITHVNPAFCRMTGWDETDLVGKVAPFPYWPRDAYPEMQRQLDMTLRGKAPSSGFELRVRRKNGTLFHARLYVSPLIDSSGRQTGWMSSMTDITEPKRAREELAAAHERFTTVLESLDAAVSVLAADEAELLFANRYYRHLFGIRPDGHLELSGGGFDRAQASSDSIDMVDAFAGLPAAALTSSAADAQEVYVESIQKWFEVRRQYIQWVDGHLAQMQIATDITTRKKAQELAHQQEEKLQFTSRLMTMGEMASSIAHELNQPLAAINNYCSGTLALVKSGRGTPETLQPALEKTAQQALRAGMIVKRIREFVKRSEPKRQPARVADIVADAVGLAEIEARKRRIRIVTEILARMPIIYVDPVLIEQVLVNLMKNAAEAMADVKPASADGVIRVVADIDAGFVDIRVIDQGPGVDEATAERLFEPFYSTKSDGMGMGLNICRSIIESHRGRLWVVNNVEPDGRISGATFHCSLPIGEPADLGQGGREASASHTVTGEL

SEQ ID NO: 9 - protein, 838 aa, unidentified
MLTDRLFARSARPSGPPAESQPSRWHHGPWWSNSYLLTPLLSILVFLVVMSLILWSLNRREQQQQEDTLFRNVAWAQQQIRLSMTGAQEQLQALSRDLASGRLDQNAFQMAVADVMQTHPEILYLNWYTAPGVQRWPTVHPPLLGQRLAKPGDAQMQDAVRGAYDEARTTRRQAYSPLIYDDFGNGFITLQTPVMRGDREYLGSIAAVFSVEGILKHDIPQELSSKYKISITDSNNRELSSTSTRPRLPRDSHYDLPLDPPGQGLTVRVYAFPQLTNLTNNTLVWLVAGLSCFVLWSLWSLWKHTRQRFEAQQALYAEAFFRRAMENSVLIGMRVLDMHGRITHVNPAFCRMTGWDESDLVGKVAPFPYWPRDAYPEMQRQLDMTLRGKAPSSGFELRVRRKNGTLFHARLYVSPLIDSSGRQTGWMSSMTDITEPKRAREELAAAHERFTTVLESLDAAVSVLAADEAELLFANRYYRHLFGIRPDGHLELSGGGFDRAQASSDSIDMVDAFAGLPAAALTSSTADAQEVYVESIQKWFEVRRQYIQWVDGHLAQMQIATDITTRKKAQELAHQQEEKLQFTSRLMTMGEMASSIAHELNQPLAAINNYCSGTLALVKSGRGTPETLQPALEKTAQQALRAGMIVKRIREFVKRSEPKRQPARVADIVADAVGLAEIEARKRRIRIVTEILARMPIIYVDPVLIEQVLMNLMKNAAEAMADVKPASADGVIRVVADIDAGFVDIRVIDQGPGVDEATAERLFEPFYSTKSDGMGMGLNICRSIIESHRGRLWVVNNVEPDGRISGATFHCSLPIGEPADLGQGGREASASHTVTGEL

SEQ ID NO: 10 - protein, 838 aa, Burkholderia arboris
MLTDRLFARSARPPGPPAESQPSRWHHGPWWSNSYLLTPLLSILVFLVVMSLILWSLNRREQQQQEDTLFRNVAWAQQQIRLSMTGAQEQLQALSRDLASGRLDQNAFQMAVADVMQTHPEILYLNWYTAPGVQRWPTVHPPLLGQRLAKPGDAQMQDAVRGAYDEARSTRRQAYSPLIYDDFGNGFITLQTPVMRGDREYLGSIAAVFSVEGILKHDIPQELSSKYKISITDANNRELSSTSTRPRLPRDSHYDLPLDPPGQGLTVRVYAFPQLTNLTNNTLVWLVAGLSCFVLWSLWSLWKHTRQRFEAQQALYAEAFFRRAMENSVLIGMRVLDMHGRITHVNPAFCRMTGWDETDLVGKVAPFPYWPRDAYPEMQRQLDMTLRGKAPSSGFELRVRRKNGTLFHARLYVSPLIDSSGRQTGWMSSMTDITEPKRAREELAAAHERFTTVLESLDAAVSVLAADEAELLFANRYYRHLFGIRPDGHLELSGGGFDRAQASSDSIDMVDAFAGLPAAALTSSTADAQEVYVESIQKWFEVRRQYIQWVDGHLAQMQIATDITTRKKAQELAHQQEEKLQFTSRLMTMGEMASSIAHELNQPLAAINNYCSGTLALVKSGRGTPETLQPALEKTAQQALRAGMIVKRIREFVKRSEPKRQPARVADIVADAVGLAEIEARKRRIRIVTEILARMPIIYVDPVLIEQVLMNLMKNAAEAMADVKPAAADGVIRVVADIDAGFVDIRVIDQGPGVDEATAERLFEPFYSTKSDGMGMGLNICRSIIESHRGRLWVVNNVEPDGRISGATFHCSLPIGEPADLGQGGREASASHTVTGEL

SEQ ID NO: 11 - protein, 838 aa, Burkholderia vietnamiensis
MLTDRLFARSARPSGPPAESQPSRWHHGPWWSNSYLLTPLLSILVFLVVMSLILWSLNRREQQQQEDTLFRNVAWAQQQIRLSMTGAQEQLQALSRDLASGRLDQNAFQMAVADVMQTHPEILYLNWYTAPGVQRWPTVHPPLLGQRLAKPGDAQMEDAVRGAYDEARSTRRQAYSPLIYDDFGNGFITLQTPVMRGDREYLGSIAAVFSVEGILKHDIPQELSSKYKISITDSNNRELSSTSTRPRLPRDSHYDLPLDPPGQGLTVRVYAFPQLTNLTNNTLVWLVAGLSCFVLWSLWSLWKHTRQRFEAQQALYAEAFFRRAMENSVLIGMRVLDMHGRITHVNPAFCRMTGWDESDLVGKVAPFPYWPRDAYPEMQRQLDMTLRGKAPSSGFELRVRRKNGTLFHARLYVSPLIDSSGRQTGWMSSMTDITEPKRAREELAAAHERFTTVLESLDAAVSVLAADEAELLFANRYYRHLFGIRPDGHLELSGGGFDRAQASSDSIDMVDAFAGLPAAALTNSTADAQEVYVESIQKWFEVRRQYIQWVDGHLAQMQIATDITTRKKAQELAHQQEEKLQFTSRLMTMGEMASSIAHELNQPLAAINNYCSGTLALVKSGRSTPETLQPALEKTAQQALRAGMIVKRIREFVKRSEPKRQPARVADIVADAVGLAEIEARKRRIRIVTEILARMPIIYVDPVLIEQVLVNLMKNAAEAMADVKAASADGVIRVVADIDAGFVDIRVIDQGPGVDEATAERLFEPFYSTKSDGMGMGLNICRSIIESHRGRLWVVNNVEPDGRISGATFHCSLPIGEPADLAQGGRESSASHTVTGEL

SEQ ID NO: 12 - protein, 838 aa, Burkholderia cepacia
MLTDRLFARSARPSGPPAESQPSRWHHGPWWSNSYLLTPLLSILVFLVVMSLILWSLNRREQQQQEDTLFRNVAWAQQQIRLSMTGAQEQLQALSRDLASGRLDQNAFQMAVADVMQTHPEILYLNWYTSPGVQRWPTVHPPLLGQRLAKPGEAQMQDAVRGAYDEARSTRRQAYSPLIYDDFGNGFITLQTPVMRGDREYLGSIAAVFSVEGILKHDIPQELSSKYKISITDANNRELSSTSTRPRLPRDSHYDLPLDPPGQGLTVRVYAFPQLTNLTNNTLVWLVAGLSCFVLWSLWSLWKHTRQRFEAQQALYAEAFFRRAMENSVLIGMRVLDMHGRITHVNPAFCRMTGWDETDLVGKVAPFPYWPRDAYPEMQRQLDMTLRGKAPSSGFELRVRRKNGTLFHARLYVSPLIDSSGRQTGWMSSMTDITEPKRAREELAAAHERFTTVLESLDAAVSVLAADEAELLFANRYYRHLFGIRPDGHLELSGGGFDRAQASSDSIDMVDAFAGLPAAALTSSTADAQEVYVESIQKWFEVRRQYIQWVDGHLAQMQIATDITTRKKAQELAHQQEEKLQFTSRLMTMGEMASSIAHELNQPLAAINNYCSGTLALVKSGRGTPETLQPALEKTAQQALRAGMIVKRIREFVKRSEPKRQPARVADIVADAVGLAEIEARKRRIRIVTEILARMPIIYVDPVLIEQVLVNLMKNAAEAMADVKPALADGVIRVIADIEAGFVDIRVIDQGPGVDEATAERLFEPFYSTKSDGMGMGLNICRSIIESHRGRLWVVNNVEPDGRISGATFHCSLPIGEPADLGQGGREASASHTVTGEL

SEQ ID NO: 13 - protein, 837 aa, Burkholderia sp.
MLTDRLFARSARPPGPPAGSQPSRWHHGPWWSNSYLLTPLLSILVFLVVMSLILWSLNRREQQQEDTLFRNVAWAQQQIRLSMTGAQEQLQALSRDLASGRLDQNAFQMAVADVMQTHPEILYLNWYTAPGVQRWPTVHPPLLGQRLAKPGDAQMQDAVRGAYDEARSTRRQAYSPLIYDDFGNGFITLQTPVMRGDREYLGSIAAVFSVEGILKHDIPQELSSKYKISITDSNNRELSSTSTRPRLPRDSHYDLPLDPPGQGLTVRVYAFPQLTNLTNNTLVWLVAGLSCFVLWSLWSLWKHTRQRFEAQQALYAEAFFRRAMENSVLIGMRVLDMHGRITHVNPAFCRMTGWDETDLVGKVAPFPYWPRDAYPEMQRQLDMTLRGKAPSSGFELRVRRKNGTLFHARLYVSPLIDSSGRQTGWMSSMTDITEPKRAREELAAAHERFTTVLESLDAAVSVLAADEAELLFANRYYRHLFGIRPDGHLELSGGGFDRAQASSDSIDMVDAFAGLPAAALTSSTADAQEVYVESIQKWFEVRRQYIQWVDGHLAQMQIATDITTRKKAQELAHQQEEKLQFTSRLMTMGEMASSIAHELNQPLAAINNYCSGTLALVKSGRGTPETLQPALEKTAQQALRAGMIVKRIREFVKRSEPKRQPARVADIVADAVGLAEIEARKRRIRIVTEILARMPIIYVDPMLIEQVLMNLMKNAAEAMADVKPASADGVIRVVADIDAGFVDIRVIDQGPGVDEATAERLFEPFYSTKSDGMGMGLNICRSIIESHRGRLWVVNNVEPDGRISGATFHCSLPIGEPADLGQGGREASASHTVTGEL

SEQ ID NO: 14 - protein, 838 aa, Burkholderia pyrrocinia
MLTDRLFARSARPSGPPAESQPSRWHHGPWWSNSYLLTPLLSILVFLVVMSLILWSLNRREQQQQEDTLFRNVAWAQQQIRLSMTGAQEQLQALSRDLASGRLDQNGFQMAVADVMQTHPEILYLNWYTSPGTQRWPTVHPPLLGQRLARPGDAQMQDAVRGAYDEARNSRRQAYAPLVYDDFGNGYITLQTPVMRGDREYLGSLAAVFSVEGILKHDIPQELSSKYKISITDANNRELSSTSSRPRLPRDSHYDLPLDPPGQGLTVRVYAFPQLTNLTNNTLVWLVAGLSCFVLWSLWSLWKHTRQRFEAQQALYAEAFFRRAMENSVLIGMRVLDMHGRITHVNPAFCRMTGWDESDLVGKVAPFPYWPRDSYPEMQRQLDMTLRGKAPSSGFELRVRRKNGTLFHARLYVSPLIDSAGRQTGWMSSMTDITEPKRAREELAAAHERFTTVLESLDAAVSVLAADEAELLFANRYYRHLFGIRPDGHLELSGGGFDRAQASSDSIDMVDTFAGLPAAALTGSTADAQEVYVESIQKWFEVRRQYIQWVDGHLAQMQIATDITTRKKAQELAHQQEEKLQFTSRLMTMGEMASSIAHELNQPLAAINNYCSGTLAMVKSGRATTETLAPALEKTAQQALRAGMIVKRIREFVKRSEPKRQPARVADIVADAVGLAEIEARKRRIRIVTEILARMPIIYVDPVLIEQVLVNLMKNAAEAMADVKPASADGVIRVIANLEAGFVDIRVIDQGPGVDEATAERLFEPFYSTKSDGMGMGLNICRSIIESHRGRLWVVNNVEADGRISGATFHCSLPIGEPADLARGGAEASASHTVTGEL

SEQ ID NO: 15 - protein, 838 aa, Burkholderia multivorans
MLTDRLFARSARPSGPPAESQPSRWHHGPWWSNSYLLTPLLSILVFLVVMSLILWSLNRREQQQQEDTLFRNVAWAQQQIRLSMTGAQEQLQALSRDLASGRLDQNAFQMAVADVMQTHPEILYLNWYTAPGVQRWPTVHPPLLGQRLAKPGDAQLQDAVRGAYDEARSSRRQAYSPLIYDDFGNGYITLQTPVMRGDRDYLGSLAAVFSVEGILKHDIPQELSSKYKISITDANNRELSSTSTRPRLPRDSHYDLPLDPPGQGLTVRVYAFPQLTNLTNNTLVWLVAGLSCFVLWSLWSLWKHTRQRFEAQQALYAEAFFRRAMENSVLIGMRVLDMHGRITHVNPAFCRMTGWDESDLVGKVAPFPYWPRDAYPEMQRQLDMTLRGKAPSSGFELRVRRKNGTLFHARLYVSPLIDSSGRQTGWMSSMTDITEPKRAREELAAAHERFTTVLESLDAAVSVLAADEAELLFANRYYHHLFGIRPDGHLELSGGGFDRAQASSDSIDMVDAFAGLPATALTNTAADAQEVYVESIQKWFEVRRQYIQWVDGHLAQMQIATDITTRKKAQELAHQQEEKLQFTSRLMTMGEMASSIAHELNQPLAAINNYCSGTLALVKSGRGTPEMLQPALEKTAQQALRAGMIVKRIREFVKRSEPKRQPARVADIVADAVGLAEIEARKRRIRIVTEILARMPIIYVDPVLIEQVLVNLMKNAAEAMADVKPPSADGVIRVVADIDAGFVDIRVIDQGPGVDEATAERLFEPFYSTKSDGMGMGLNICRSIIESHRGRLWVVNNVEPDGRISGATFHCSLPIGEPADLGQGGSETSASHTVTGEL

SEQ ID NO: 16 - protein, 838 aa, Burkholderia cenocepacia
MLTDRLFARSARPPGPPAESQPSRWHHGPWWSNSYLLTPLLSILVFLVVMSLILWSLNRREQQQQEDTLFRNVAWAQQQIRLSMTGAQEQLQALSRDLASGRLDQNAFQMAVADVMQTHPEILYLNWYTAPGVQRWPTVHPPLLGQRLAKPGDAQMQDAVRGAYDEARSTRRQAYSPLIYDDFGNGFITLQTPVMRGDRDYLGSIAAVFSVEGILKHDIPQELSSKYKISITDSNNRELSSTSTRPRLPRDSHYDLPLDPPGQGLTVRVYAFPQLTNLTNNTLVWLVAGLSCFVLWSLWSLWKHTRQRFEAQQALYAEAFFRRAMENSVLIGMRVLDMHGRITHVNPAFCRMTGWDETDLVGKVAPFPYWPRDAYPEMQRQLDMTLRGKAPSSGFELRVRRKNGTLFHARLYVSPLIDSSGRQTGWMSSMTDITEPKRAREELAAAHERFTTVLESLDAAVSVLAADEAELLFANRYYRHLFGIRPDGHLELSGGGFDRAQASSDSIDMVDAFAGLPAAALTSSTADAQEVYVESIQKWFEVRRQYIQWVDGHLAQMQIATDITTRKKAQELAHQQEEKLQFTSRLMTMGEMASSIAHELNQPLAAINNYCSGTLALVKSGRGTPETLQPALEKTAQQALRAGMIVKRIREFVKRSEPKRQPARVADIVADAVGLAEIEARKRRIRIVTEILARMPIIYVDPVLIEQVLMNLMKNAAEAMADVKPASADGVIRVVADIDAGFVDIRVIDQGPGVDEATAERLFEPFYSTKSDGMGMGLNICRSIIESHRGRLWVVNNVEPDGRISGATFHCSLPIGEPADLGQGGREASASHTVTGEL

SEQ ID NO: 17 - protein, 836 aa, Burkholderia thailandensis
MLTDRLFARSARPSGSPAESQPSRWHHGPWWSNSYLLTPLLSILVFLVVMSLILWSLNRREEQQQEDTLFRNVAWAQQQIRLSMTSAQEQLQAFSRDIAAGRIDEHAFQTTVGDVMQAHPEILYLNWYTSPGTKRWPTMQLPVLGQRLAKPGDAQMDEVVRGAYAQARSTRRQSYSPLVYDDFGNGYLTLQTPVIRDREYLGSIAAVFSVEGILKHDIPPELSAKYKISITDANNRELASTSSRPRLPRDAHYDLPLDPPGQGLTVRVYAYPQTTNLTNNTLVWLVAGLSCFVLWSLWSLWKHTRQRFEAQQALYAEAFFRRAMENSVLIGMRVLDMHGRITHVNPAFCRMTGWDESDLVGKTAPFPYWPRDAYPEMQRQLDMTLRGKAPSSGFELRVRRKDGSLFHARLYVSPLIDSAGRQTGWMSSMTDITEPKRAREELAAAHERFTTVLESLDAAVSVLAADEAELLFANRYYRHLFGIRPDGHLELSGGGFDTAQASSDSIDMVDAYAGLPAAALTESTADAQEVYVESIQKWFEVRRQYIQWVDGHLAQMQIATDITTRKKAQELAHQQEEKLQFTSRLMTMGEMASSIAHELNQPLAAINNYCSGTLALVKSGRASPETLAPALEKTAQQALRAGMIVKRIREFVKRSEPKRQPSRVADIVADAVGLAEIEARKRRIRIVTEIRARMPIIYVDPVLIEQVLVNLMKNAAEAMQEARPQAENGVIRVVADLESGFVDIRVIDQGPGVDEATAERLFEPFYSTKSDGMGMGLNICRSIIESHRGRLWVVNNVEPDGFVSGATFHCSLPIGEPEDLGHGSETSPSQTVTGEI

SEQ ID NO: 18 - DNA, 3240 bp, Bradyrhizobium japonicum
  source 1..3240: mol_type = genomic DNA
gcatgcccgccgcgattcctggtggccgcgcggaattgcgcctccagagccgccagccgtcgcctgacggacgcttcgccctcgtcgatcagtccctgggcggcggcgccgtcggtgaaatagagcggcggggcgaactgcgccgcggcaaccccgacgatggcggcctcgaatcgctcggcgagctcgcccgcgacctgaagacgcgcctcgttggactggtcgagcgccaagctgaccatcacggtcgcgtatgtcattagaattctccgggcaatgaactgctatgaaatctagctgtgccagcgcgaggcaggatgagatagatcaaaccgtcgcccacccgggaccaccggcattacgccaatggaatcaaacatccgaacttgcctccggcccgggcttgagcgaaattaccgccgtggttgcggcacctgggcccgtggcacgagaatgggagtgacaccttggcgccgacccgcgtaacgcatccgccggatgacggccggggcgagcatttccgggtcaggatcgagggattcggcgtcggcacctgggatctcgacctcaagacctgggcgctggactggtcggataccgcgcggaccctgctcggaatcgggcaggaccagccggcgagctacgacctcttcctgtcacgcctcgagcccgacgaccgcgagcgcgtggagagcgcgatcaagcgcgtctccgaacgcggtggcggcttcgacgtgtccttcagggtcgccggcacctccaacgcaggacagtggattcgcgcccgggccggactcattcgggacgaagccggcaccgcccgccatctcagcggcatctttctcgatatcgacgaggagaagcaggtcgagggtgcgctccgtacccgcgagacccacctccgctcgatcctccacacaattcccgacgccatgatcgtcatcgacggccacggcatcatccagctgttcagcaccgccgccgagcgcctgttcggctggtccgagctcgaggcgatcggccagaacgtcaacatcctgatgccggagcccgaccgctcccggcatgacagctacatttcgcgttaccgcaccacgagcgatccccacatcatcggcatcgggcgcatcgtgaccggcaagcgccgcgacggcaccaccttcccgatgcacctgtcgatcggcgagatgcagtccggcggcgagccctattttaccggtttcgtccgcgatctcaccgagcaccagcagacccaggcgcgtctccaggaactgcaatccgagctcgtccacgtctccaggctgagcgccatgggcgaaatggcgtccgcgctcgcgcacgagctcaaccagccgctggcggcgatcagcaactacatgaagggctcgcggcggctgcttgccggcagcagtgatccgaacacaccgaaggtcgaaagcgccctggaccgcgccgccgagcaggcgctgcgcgccggccagatcatccggcgcctgcgcgacttcgttgcccgcggcgaatcggagaagcgggtcgagagtctctccaagctgatcgaggaggccggcgcgctcgggcttgccggcgcgcgcgagcagaacgtgcagctccgcttcagtctcgatccgggcgccgatctcgttctcgccgaccgggtgcagatccagcaggtcctggtcaacctgttccgcaacgcgctggaagcgatggctcagtcgcagcgacgcgagctcgtcgtcaccaacacccccgccgccgacgacatgatcgaggtcgaagtgtccgacaccggcagcggtttccaggacgacgtcattccgaacctgtttcagactttcttcaccaccaaggacaccggcatgggcgtgggactgtccatcagccgctcgatcatcgaagctcacggcgggcgcatgtgggccgagagcaacgcatcgggcggggcgaccttccgcttcaccctcccggcagccgacgagaattgatgcatgacgaccaagggacatatctacgtcatcgacgacgacgcggcgatgcgggattcgctgaatttcctgctggattctgccggcttcggcgtcacgctgtttgacgacgcgcaagcctttctcgacgccctgccgggtctctccttcggctgcgtcgtctccgacgtgcgcatgccgggccttgacggcatcgagctgttgaagcggatgaaggcgcagcaaagcccctttccgatcctcatcatgaccggtcacggcgacgtgccgctcgcggtcgaggcgatgaagttaggggcggtcgactttctggaaaagcctttcgaggacgaccgcctcaccgccatgatcgaatcggcgatccgccaggccgagccggccgccaagagcgaggccgtcgcgcaggatatcgccgcccgcgtcgcctcgttgagccccagggagcgccaggtcatggaagggctgatcgccggcctttccaacaagctgatcgcccgcgagtacgacatcagcccgcgcaccatcgaggtgtatcgggccaacgtcatgaccaagatgcaggccaacagcctttcggagctggttcgcctcgcgatgcgcgccggcatgctcaacgattgagccaagtcaaggcccttgccggatgctgtgctagccatgcagcatgatcgaggtcagttcacatcacgagcggctgccgtcctccacaaaacccaccgtctttgtggtcgatgatgacgccgcagtcctgggatccctgcggttcctgctggaaaccgacggctttgccgtgcggaccttcaggagcggcacggcgctgctcaatgccggcggcgcgcccggggccgactgctacgtgatcgactacaagatgcccgacatcaatggaatcgagctcgccagccgcctgcgtaaatcggacggcgaaacgcccgtgatcctgatcaccggctatccggacgaaaacatctcgacccgggccgccgcggccggcgtaaaagacgtggttttgaagccgcttctcgacgaaaacctgctcaagcgtatccgccgcgccatccaggaccggcctcgggcatgacctacggggttctacgtaaggcaccccccttaagatatcgctcgaaattttcgaacctcccgataccgcgtaccaatgcgtcatcacaacggagatggcgcagatgctgacccagacactcaagacccaggtgatcaacacccaaatcggtggcaagattgccccgccccatca

SEQ ID NO: 19 - protein, 212 aa, unidentified
MNSPVTTTQETVFVVDDDEAVRDSLRWLLEANGYRVQCFSSAEQFLDAYQPAQQAGQIACLILDVRMSGMSGLELQERLIADNAALPIIFVTGHGDVPMAVSTMKKGAMDFIEKPFDEAELRKLVERMLDKARSESKSVQEQRAATERLSKLTAREQQVLERIIAGRLNKQIADDLGISIKTVEAHRANIMEKLNVNTVADLLRLALSKKQA

SEQ ID NO: 20 - protein, 212 aa, Burkholderia dolosa
MNSPVTTTQETVFVVDDDEAVRDSLRWLQEANGYRVQCFSSAEQFLDAYQPAQQAGQIACLILDVRMSGMSGLELQERLIADNAALPIIFVTGHGDVPMAVSTMKKGAMDFIEKPFDEAELRKLVERMLDKARSESKSVQEQRAASERLSKLTAREQQVLERIIAGRLNKQIADDLGISIKTVEAHRANIMEKLNVNTVADLLRLALSKKQA

SEQ ID NO: 21 - protein, 212 aa, Burkholderia vietnamiensis
MNSPVTTTQETVFVVDDDEAVRDSLRWLLEANGYRVQCFSSAEQFLDAYQPAQQAGQIACLILDVRMSGMSGLELQERLIADNAALPIIFVTGHGDVPMAVSTMKKGAMDFIEKPFDEAELRKLVERMLDKARSESKSVQEQRAASERLSKLTAREQRVLERIIAGRLNKQIADDLGISIKTVEAHRANIMEKLNVNTVADLLRLALSKKQA

SEQ ID NO: 22 - protein, 212 aa, Burkholderia cepacia
MNSPVTTTQETVFVVDDDEAVRDSLRWLLEANGYRVQCFSSAEQFLDAYQPAQQAGQVACLILDVRMSGMSGLELQERLIADNAALPIIFVTGHGDVPMAVSTMKKGAMDFIEKPFDEAELRKLVERMLDKARSESKSVQEQRAASERLSKLTAREQQVLERIIAGRLNKQIADDLGISIKTVEAHRANIMEKLNVNTVADLLRLALSKKQA

SEQ ID NO: 23 - protein, 212 aa, Burkholderia multivorans
MNSPVTTTQETVFVVDDDEAVRDSLRWLLEANGYRVQCFSSAEQFLDAYQPAQQAGQIACLILDVRMSGMSGLELQERLIADNAALPIIFVTGHGDVPMAVSTMKKGAMDFVEKPFDEAELRKLVERMLDKARSESKSVQEQRAASERLSKLTAREQQVLERIIAGRLNKQIADDLGISIKTVEAHRANIMEKLNVNTVADLLRLALSKKPA

SEQ ID NO: 24 - protein, 212 aa, Burkholderia multivorans
MNSPVTTTQETVFVVDDDEAVRDSLRWLLEANGYRVQCFSSAEQFLDAYQPAQQAGQIACLILDVRMSGMSGLELQERLIADNAALPIIFVTGHGDVPMAVSTMKKGAMDFIEKPFDEAELRKLVERMLDKARSESKSVQEQRAASERLSKLTAREQQVLERIIAGRLNKQIADDLGISIKTVEAHRANIMEKLNVNTVADLLRLALSKNPA

SEQ ID NO: 25 - protein, 212 aa, Burkholderia cenocepacia
MNSPVTTTQETVFVVDDDEAVRDSLRWLLEANGYRVQCFSSAEQFLDAYQPAQQAGQIACLILDVRMSGMSGLELQERLIADNAALPIIFVTGHGDVPMALSTMKKGAMDFIEKPFDEAELRKLVERMLDKARSESKSVQEQRAASERLSKLTAREQQVLERIIAGRLNKQIADDLGISIKTVEAHRANIMEKLNVNTVADLLRLALSKKQA

SEQ ID NO: 26 - protein, 212 aa, Burkholderia vietnamiensis
MNSPVTTTQETVFVVDDDEAVRDSLRWLLEANGYRVQCFSSAEQFLDAYQPAQQAGQIACLILDVRMSGMSGLELQERLIADNAALPIIFVTGHGDVPMAVSTMKKGAMDFIEKPFDEAELRKLVERMLDKARSESKSVQEQRAASERVSKLTAREQQVLERIIAGRLNKQIADDLGISIKTVEAHRANIMEKLNVNTVADLLRLALSKKQA

SEQ ID NO: 27 - protein, 212 aa, Burkholderia cenocepacia
MNSPVTTTQETVFVVDDDEAVRDSLRWLLEANGYRVQCFSSAEQFLDAYQPAQQAGQIACLILDVRMSGMSGLELQERLIADNAALPIIFVTGHGDVPMAVSTMKKGAMDFIEKPFDEAELRKLVERMLDKARSESKSVQEQRAASERLSKLTAREQQVLERIIAGRLNKQIADVLGISIKTVEAHRANIMEKLNVNTVADLLRLALSKKQA

SEQ ID NO: 28 - protein, 212 aa, unidentified
MNSPVTTTQETVFVVDDDEAVRDSLRWLLEANGYRVQCFSSAEQFLDAYQPAQQAGQIACLILDVRMSGMSGLELQERLIADNAALPIIFVTGHGDVPMAVSTMKKGAMDFIEKPFDEAELRKLVERMLDKARSESKSVQEQRAASERLSKLTAREQQVLERIIAGRLNKQIADDLGISIKTVEAHRANIMEKLNVNTVADLLRLALSKKQA

SEQ ID NO: 29 - protein, 212 aa, Burkholderia latens; identical to SEQ ID NO: 28

SEQ ID NO: 30 - protein, 212 aa, Burkholderia ambifaria
MNSPVTTTQETVFVDDDDEAVRDSLRWLLEANGYRVQCFSSAEQFLDAYQPAQQAGQIACLILDVRMSGMSGLELQERLIADNAALPIIFVTGHGDVPMAVSTMKKGAMDFIEKPFDEAELRKLVERMLDKARSESKSVQEQRAASERLSKLTAREQQVLERIIAGRLNKQIADDLGISIKTVEAHRANIMEKLNVNTVADLLRLALSKKQA

SEQ ID NO: 31 - protein, 212 aa, Burkholderia anthina
MNSPVTTTQETVFVVDDDEAVRDSLRWLLEANGYRVQCFSSAEQFLDAYQPAQQAGQIACLILDVRMSGMSGLELQERLIADNAALPIIFVTGHGDVPMTVSTMKKGAMDFIEKPFDEAELRKLVERMLDKARSESKSVQEQRAASERLSKLTAREQQVLERIIAGRLNKQIADDLGISIKTVEAHRANIMEKLNVNTVADLLRLALSKKQA

SEQ ID NO: 32 - protein, 212 aa, Burkholderia contaminans
MNSPVTTTQETVFVVDDDEAVRDSLRWLLEANGYRVQCFSSAEQFLDAYQPAQQAGQIACLILDVRMSGMSGLELQERLIADNAALPIIFVTGHGDVPMAVSTMKKGAMDFIEKPFDEAELRKLVERMLDKARSESKSVQEQRAASERLSKLTAREQQVHERIIAGRLNKQIADDLGISIKTVEAHRANIMEKLNVNTVADLLRLALSKKQA

SEQ ID NO: 33 - protein, 212 aa, Burkholderia metallica
MNSPVTTTQETVFVVDDDEAVRDSLRWLLEANGYRVQCFSSAEQFIDAYQPAQQAGQIACLILDVRMSGMSGLELQERLIADNAALPIIFVTGHGDVPMAVSTMKKGAMDFIEKPFDEAELRKLVERMLDKARSESKSVQEQRAASERLSKLTAREQQVLERIIAGRLNKQIADDLGISIKTVEAHRANIMEKLNVNTVADLLRLALSKKQA

SEQ ID NO: 34 - protein, 212 aa, Burkholderia diffusa; identical to SEQ ID NO: 28

SEQ ID NO: 35 - protein, 212 aa, Burkholderia pyrrocinia; identical to SEQ ID NO: 28

SEQ ID NO: 36 - protein, 212 aa, Burkholderia seminalis; identical to SEQ ID NO: 28

SEQ ID NO: 37 - protein, 215 aa, Burkholderia sp.
MNEALHSTPDASVVAIVDDDEAVRDGLALLLHSVGLATRCYADAQAFLVDADDAALGCVLLDIRMPGMSGLDALDELRARRDLPAIVMTGHGNIDACRRAFKRGALDFLRKPVDDDELIDAVQQAIRRHAAQREQGAHGGADPARAARIATLSAREREVLDGIVRGWSNKEIARELGLSPRTVETYRANVFDKLQAASLVELVREYAAFAGAASP

SEQ ID NO: 38 - protein, 212 aa, Burkholderia pseudomallei
MNSPVTTSQETVFVVDDDEAVRDSLRWLLEANGYRVQCFSSAEQFLDAYQPAQQAGQIACLILDVRMSGMSGLELQERLIAENAALPIIFVTGHGDVPMAVSTMKKGAMDFIEKPFDEAELRKLVERMLEKARNESKSVQEQRAASERLSKLTAREQQVLERIIAGRLNKQIADDLGISIKTVEAHRANIMEKLNVNTVADLLRLALSKKQA

SEQ ID NO: 39 - protein, 212 aa, Burkholderia thailandensis
MNSPVTTSQETVFVVDDDEAVRDSLRWLLEANGYRVQCFSSAEQFLEAYQPAQQAGQIACLILDVRMSGMSGLELQERLIAENAALPIIFVTGHGDVPMAVSTMKKGAMDFIEKPFDEAELRKLVERMLDKARSESKSVQEQRAASERLSKLTAREQQVLERIIAGRLNKQIADDLGISIKTVEAHRANIMEKLNVNTVADLLRLALSKKQA